SEQUENCE LISTING (106 sequences)

SEQ ID NO: 1 - protein, 538 aa, Mumps virus
MKAFSVTCLGFAVFSSSICVNINILQQIGYIKQQVRQLSYYSQSSSSYIVVKLLPNIQPTDNSCEFKSVTQYNKTLSNLLLPIAENINNIASPSPGSRRHKRFAGIAIGIAALGVATAAQVTAAVSLVQAQTNARAIAAMKNSIQATNRAIFEVKEGTQQLAIAVQAIQDHINTIMNTQLNNMSCQILDNQLATYLGLYLTELTTVFQPQLINPALSPISIQALRSLLGSMTPAVVQATLSTSISAAEILSAGLMEGQIVSVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTTCQTLSLDGLDFSIVSLSNITYAENLTISLSQTINTQPIDISTELSKVNASLQNAVKYIKESNHQLQSVSVNSKIGAIIVAALVLSILSIIISLLFCCWAYIATKEIRRINFKTNHINTISSSVDDLIRY

SEQ ID NO: 2 - protein, 513 aa, synthetic construct
  REGION 1..513: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLACAVQAIQDHINTIMNTQLNNMSCQILDNQLATSLGLYLTELTTVFQPQLTNPALSPISIQALRSLLGSMTPACVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSVSVNSKIIEDKIEEILSKIYHIENEIARIKKLIGEAP

SEQ ID NO: 3 - protein, 513 aa, synthetic construct
  REGION 1..513: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTCFQPQLTNPALSPISIQCLRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSVSVNSKIIEDKIEEILSKIYHIENEIARIKKLIGEAP

SEQ ID NO: 4 - protein, 513 aa, synthetic construct
  REGION 1..513: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAECINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTVFQPQLTNPCLSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSVSVNSKIIEDKIEEILSKIYHIENEIARIKKLIGEAP

SEQ ID NO: 5 - protein, 513 aa, synthetic construct
  REGION 1..513: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTVFQCQLTNCALSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSVSVNSKIIEDKIEEILSKIYHIENEIARIKKLIGEAP

SEQ ID NO: 6 - protein, 513 aa, synthetic construct
  REGION 1..513: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVCEGTQQCAIAVQAIQDHINTIMNTQLNNMSCQILDNQLATSLGLYLTELTTVFQPQLTNPALSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSVSVNSKIIEDKIEEILSKIYHIENEIARIKKLIGEAP

SEQ ID NO: 7 - protein, 513 aa, synthetic construct
  REGION 1..513: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIACQAIQDHINTIMNTQLNNMSCQILDNQLATSLGLYLTELTTVFQPQLTNPALSPISIQALRSLLGSCTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSVSVNSKIIEDKIEEILSKIYHIENEIARIKKLIGEAP

SEQ ID NO: 8 - protein, 513 aa, synthetic construct
  REGION 1..513: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTVFQPQLTNPALSPISCQALRSLLGSMTPAVVQATLSTSISAAEILSAGLCEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSVSVNSKIIEDKIEEILSKIYHIENEIARIKKLIGEAP

SEQ ID NO: 9 - protein, 513 aa, synthetic construct
  REGION 1..513: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMPCQILDNQLATSLGLYLTELTTVFQPQLTNPALSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSVSVNSKIIEDKIEEILSKIYHIENEIARIKKLIGEAP

SEQ ID NO: 10 - protein, 506 aa, synthetic construct
  REGION 1..506: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLACAVQAIQDHINTIMNTQLNNMSCQILDNQLATSLGLYLTELTTVFQPQLTNPALSPISIQALRSLLGSMTPACVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSIEDKIEEILSKIYHIENEIARIKKLIGEAP

SEQ ID NO: 11 - protein, 506 aa, synthetic construct
  REGION 1..506: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTCFQPQLTNPALSPISIQCLRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSIEDKIEEILSKIYHIENEIARIKKLIGEAP

SEQ ID NO: 12 - protein, 506 aa, synthetic construct
  REGION 1..506: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAECINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTVFQPQLTNPCLSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSIEDKIEEILSKIYHIENEIARIKKLIGEAP

SEQ ID NO: 13 - protein, 506 aa, synthetic construct
  REGION 1..506: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTVFQCQLTNCALSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSIEDKIEEILSKIYHIENEIARIKKLIGEAP

SEQ ID NO: 14 - protein, 506 aa, synthetic construct
  REGION 1..506: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVCEGTQQCAIAVQAIQDHINTIMNTQLNNMSCQILDNQLATSLGLYLTELTTVFQPQLTNPALSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSIEDKIEEILSKIYHIENEIARIKKLIGEAP

SEQ ID NO: 15 - protein, 506 aa, synthetic construct
  REGION 1..506: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIACQAIQDHINTIMNTQLNNMSCQILDNQLATSLGLYLTELTTVFQPQLTNPALSPISIQALRSLLGSCTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSIEDKIEEILSKIYHIENEIARIKKLIGEAP

SEQ ID NO: 16 - protein, 506 aa, synthetic construct
  REGION 1..506: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTVFQPQLTNPALSPISCQALRSLLGSMTPAVVQATLSTSISAAEILSAGLCEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSIEDKIEEILSKIYHIENEIARIKKLIGEAP

SEQ ID NO: 17 - protein, 506 aa, synthetic construct
  REGION 1..506: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMPCQILDNQLATSLGLYLTELTTVFQPQLTNPALSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSIEDKIEEILSKIYHIENEIARIKKLIGEAP

SEQ ID NO: 18 - protein, 499 aa, synthetic construct
  REGION 1..499: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLACAVQAIQDHINTIMNTQLNNMSCQILDNQLATSLGLYLTELTTVFQPQLTNPALSPISIQALRSLLGSMTPACVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKEIEDKIEEILSKIYHIENEIARIKKLIGEAP

SEQ ID NO: 19 - protein, 499 aa, synthetic construct
  REGION 1..499: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTCFQPQLTNPALSPISIQCLRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKEIEDKIEEILSKIYHIENEIARIKKLIGEAP

SEQ ID NO: 20 - protein, 499 aa, synthetic construct
  REGION 1..499: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAECINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTVFQPQLTNPCLSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKEIEDKIEEILSKIYHIENEIARIKKLIGEAP

SEQ ID NO: 21 - protein, 499 aa, synthetic construct
  REGION 1..499: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTVFQCQLTNCALSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKEIEDKIEEILSKIYHIENEIARIKKLIGEAP

SEQ ID NO: 22 - protein, 499 aa, synthetic construct
  REGION 1..499: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVCEGTQQCAIAVQAIQDHINTIMNTQLNNMSCQILDNQLATSLGLYLTELTTVFQPQLTNPALSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKEIEDKIEEILSKIYHIENEIARIKKLIGEAP

SEQ ID NO: 23 - protein, 499 aa, synthetic construct
  REGION 1..499: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIACQAIQDHINTIMNTQLNNMSCQILDNQLATSLGLYLTELTTVFQPQLTNPALSPISIQALRSLLGSCTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKEIEDKIEEILSKIYHIENEIARIKKLIGEAP

SEQ ID NO: 24 - protein, 499 aa, synthetic construct
  REGION 1..499: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTVFQPQLTNPALSPISCQALRSLLGSMTPAVVQATLSTSISAAEILSAGLCEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKEIEDKIEEILSKIYHIENEIARIKKLIGEAP

SEQ ID NO: 25 - protein, 499 aa, synthetic construct
  REGION 1..499: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMPCQILDNQLATSLGLYLTELTTVFQPQLTNPALSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKEIEDKIEEILSKIYHIENEIARIKKLIGEAP

SEQ ID NO: 26 - protein, 506 aa, synthetic construct
  REGION 1..506: note = Recombinant F protein
MKAFSVTCLSFAVFSSSICVNINILQQIGYIKQQVRQLSYYSQSSSSYVVVKLLPNIQPTDNSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTCFQPQLINPALSPISIQCLRSLLGSMTPAVVQATLSTSISAAEILSAGLMEGQIVSVLLDEMQMIVKINVPTIVTQSNALVIDFYSISSFINNQESIIQLPDRILEIGNEQWRYPAKNCKSTRHHIFCQYNEAERLSLETKLCLAGNISACVFSPIAGSYMRRFVALDGTIVANCRSLTCLCKSPSYPIYQPDHHAVTTIDLTSCQTLSLDGLDFSIVSLSNITYAENLTISLSQTINTQPIDISTELSKVNASLQNAVKYIKESNHQLQSIEDKIEEILSKIYHIENEIARIKKLIGEAP

SEQ ID NO: 27 - protein, 966 aa, synthetic construct
  REGION 1..966: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTCFQPQLTNPALSPISIQCLRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSIEDKIEEILSKIYHIENEIARIKKLIGEAPGSGGGGGGNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTIT

SEQ ID NO: 28 - protein, 981 aa, synthetic construct
  REGION 1..981: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTCFQPQLTNPALSPISIQCLRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSIEDKIEEILSKIYHIENEIARIKKLIGEAPGSGYIPEAPRDGQAYVRKDGEWVLLSTFLGSGGGGGGFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPADMQSWVPLSTDDPVIDRLYLSSHRGVIADNQAKWAVPTTRTDDKLRMETCFQQACKGKIQTLCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGEDCHAPTYLPAEVDGDVKLSSNLVILPGQDLQYVLATYDTSRVEHAVVYYVYSPGRSFSYFYPFRLPIKGVPIELQVECFTWDQKLWCRHFCVLADSESGGHITHSGMVGMGVSCTVTREDGTNRR

SEQ ID NO: 29 - protein, 1006 aa, synthetic construct
  REGION 1..1006: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQDHINTIMNTQLNNMSCQILDNQLATSLGLYLTELTTCFQPQLTNPALSPISIQCLRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSIEDKIEEILSKIYHIENEIARIKKLIGEAPGSGYIPEAPRDGQAYVRKDGEWVLLSTFLGSGGGGGGADVAAEELMNALVNSTLLEARATNQFLAVSKGNCSGPTTIRGQFSNMSLSLLDLYLSRGYNVSSIVTMTSQGMYGGTYLVGKPNLSSKGSELSQLSMHRVFEVGVIRNPGLGAPVFHMTNYFEQPVSNDFSNCMVALGELKFAALCHREDSITIPYQGSGKGVSFQLVKLGVWKSPTDMRSWVPLSTDDPVIDRLYLSSHRGVIADNQAKWAVPTTRTDDKLRMETCFQQACKGKNQALCENPEWAPLKDNRIPSYGVLSVNLSLTVELKIKIASGFGPLITHGSGMDLYKTNHNNVYWLTIPPMKNLALGVINTLEWIPRFKVSPNLFTVPIKEAGEDCHAPTYLPAEVDGDVKLSSNLVILPGQDLQYVLATYDTSRVEHAVVYYVYSPSRSFSYFYPFRLPIKGVPIELQVECFTWDKKLWCRHFCVLADSESGGHITHSGMVGMGVSCTVTREDGTNRR

SEQ ID NO: 30 - protein, 452 aa, Mumps virus
NIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTIT

SEQ ID NO: 31 - protein, 438 aa, Measles virus
FLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPADMQSWVPLSTDDPVIDRLYLSSHRGVIADNQAKWAVPTTRTDDKLRMETCFQQACKGKIQTLCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGEDCHAPTYLPAEVDGDVKLSSNLVILPGQDLQYVLATYDTSRVEHAVVYYVYSPGRSFSYFYPFRLPIKGVPIELQVECFTWDQKLWCRHFCVLADSESGGHITHSGMVGMGVSCTVTREDGTNRR

SEQ ID NO: 32 - protein, 463 aa, Measles virus
ADVAAEELMNALVNSTLLEARATNQFLAVSKGNCSGPTTIRGQFSNMSLSLLDLYLSRGYNVSSIVTMTSQGMYGGTYLVGKPNLSSKGSELSQLSMHRVFEVGVIRNPGLGAPVFHMTNYFEQPVSNDFSNCMVALGELKFAALCHREDSITIPYQGSGKGVSFQLVKLGVWKSPTDMRSWVPLSTDDPVIDRLYLSSHRGVIADNQAKWAVPTTRTDDKLRMETCFQQACKGKNQALCENPEWAPLKDNRIPSYGVLSVNLSLTVELKIKIASGFGPLITHGSGMDLYKTNHNNVYWLTIPPMKNLALGVINTLEWIPRFKVSPNLFTVPIKEAGEDCHAPTYLPAEVDGDVKLSSNLVILPGQDLQYVLATYDTSRVEHAVVYYVYSPSRSFSYFYPFRLPIKGVPIELQVECFTWDKKLWCRHFCVLADSESGGHITHSGMVGMGVSCTVTREDGTNRR

SEQ ID NO: 33 - protein, 30 aa, synthetic construct
  REGION 1..30: note = GCN4 trimerization domain
IEDKIEEILSKIYHIENEIARIKKLIGEAP

SEQ ID NO: 34 - protein, 27 aa, synthetic construct
  REGION 1..27: note = T4 Fibritin Trimerization domain
GYIPEAPRDGQAYVRKDGEWVLLSTFL

SEQ ID NO: 35 - protein, 59 aa, synthetic construct
  REGION 1..59: note = GCN4/T4 Fibritin trimerization domain
IEDKIEEILSKIYHIENEIARIKKLIGEAPGSGYIPEAPRDGQAYVRKDGEWVLLSTFL

SEQ ID NO: 36 - protein, 550 aa, Measles virus
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYLIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPILLERLDVGTNLGNAIAKLEDAKELLESSDQILRSMKGLSSTCIVYILIAVCLGGLIGIPALICCCRGRCNKKGEQVGMSRPGLKPDLTGTSKSYVRSL

SEQ ID NO: 37 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDCLNAVTQNIRPVQSVASSRRHGGGAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPCSAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 38 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAICQAGQECILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 39 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAIRQAGQEMICAVQGVQDYINNELIPSMNQLSCDLIGQKLGLKLLRYYTEILSLFGPSLRDPISAEISIQALSYALGGDINKCLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 40 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAIRQAGQEMILACQGVQDYINNELIPSMNQLSCDLIGQKLGLKLLRYYTEILSLFGPSLRDPISAEISIQALSYALGGCINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 41 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSCFGPSLRDPISAEISIQCLSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 42 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGCSLRDCISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 43 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAIRQAGQEMILAVQGVQDYINNELIPSMNQLPCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 44 - protein, 24 aa, synthetic construct
  REGION 1..24: note = transmembrane domain
GAIIVAALVLSILSIIISLLFCCW

SEQ ID NO: 45 - protein, 163 aa, synthetic construct
  REGION 1..163: note = Ferritin subunit
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 46 - protein, 154 aa, synthetic construct
  REGION 1..154: note = lumazine synthase subunit
MQIYEGKLTAEGLRFGIVASRFNHALVDRLVEGAIDCIVRHGGREEDITLVRVPGSWEIPVAAGELARKEDIDAVIAIGVLIRGATPHFDYIASEVSKGLADLSLELRKPITFGVITADTLEQAIERAGTKHGNKGWEAALSAIEMANLFKSLR

SEQ ID NO: 47 - protein, 265 aa, synthetic construct [sequence omitted]

SEQ ID NO: 48 - protein, 265 aa, synthetic construct [sequence omitted]

SEQ ID NO: 49 - protein, 617 aa, Measles virus
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMVGMGVSCTVTREDGTNRR

SEQ ID NO: 50 - protein, 582 aa, Mumps virus
MEPSKFFTISDSATFAPGPVSNAADKKTFRTCFRILVLSVQAVTLILVIVTLGELVRMINDQGLSNQLSSITDKIRESATMIASAVGVMNQVIHGVTVSLPLQIEGNQNQLLATLATICASQKQVSNCSTNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTIT

SEQ ID NO: 51 - protein, 506 aa, synthetic construct
  REGION 1..506: note = Recombinant F protein
MKVSLVTCLGFAVFSFSICVNINILQQIGYIKQQVRQLSYYSQSSSSYIVVKLLPNIQPTDNSCEFKSVTQYNKTLSNLLLPIAENINNIASPSPGSRRHGGGAGIAIGIAALGVATAAQVTAAVSLVQAQTNARAIAAMKNSIQATNRAVFEVKEGTQQLAIAVQAIQNHINTIMNTQLNNMSCQILDNQLATSLGLYLTELTTCFQPQLINPALSPISIQCLRSLLGSMTPAVVQATLSTSISAAEILSAGLMEGQIVSVLLDEMQMIVKINIPTIVTQSNALVIDFYSISSFINGQESIIQLPDRILEIGNEQWSYPAKNCKLTRHNIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELIKVNASLQNAVKYIKESNHQLQSIEDKIEEILSKIYHIENEIARIKKLIGEAP

SEQ ID NO: 52 - protein, 516 aa, synthetic construct
  REGION 1..516: note = Recombinant F protein
MKAFSVTCLSFAVFSSSICVNINILQQIGYIKQQVRQLSYYSQSSSSYIVVKLLPNIQPTDDSCEFKSVTQYNKTLSNLLLPIAENINNIASPSPGSRRHGGGAGIAIGIAALGVATAAQVTAAVSLVQAQTNARAIAAMKNSIQATNRAVFEVCEGTQQCAIAVQAIQDHINTIMNTQLNNMSCQILDNQLATSLGLYLTELTTVFQPQLTNPALSPISIQALRSLLGSMTPAVVQATLSTSISAAEILSAGLMEGQIISVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTACQTLSLDGLDFSIVSLSNITYAENLTISLSQTINTQPIDISTELSKVNASLQNAVKYIKESNHQLQSVSVNSKIGAIIEDKIEEILSKIYHIENEIARIKKLIGEAP

SEQ ID NO: 53 - protein, 513 aa, synthetic construct
  REGION 1..513: note = Recombinant F protein
MYSMQLASCVTLTLVLLVNSQIHWGNLSKIGVVGIGSASYKVMTCSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSICYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 54 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSCLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARICHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 55 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAIRQAGQEMILAVQGVQDYINNELIPSMNQLSCDLIGQKLGLKLLRYYTCILSLFGPSLRDPISAEISIQALSCALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 56 - protein, 959 aa, synthetic construct
  REGION 1..959: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAICQAGQECILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAPGSGGGGGGFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPADMQSWVPLSTDDPVIDRLYLSSHRGVIADNQAKWAVPTTRTDDKLRMETCFQQACKGKIQTLCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGEDCHAPTYLPAEVDGDVKLSSNLVILPGQDLQYVLATYDTSRVEHAVVYYVYSPGRSFSYFYPFRLPIKGVPIELQVECFTWDQKLWCRHFCVLADSESGGHITHSGMVGMGVSCTVTREDGTNRR

SEQ ID NO: 57 - protein, 973 aa, synthetic construct
  REGION 1..973: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAICQAGQECILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAPGSGGGGGGNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTIT

SEQ ID NO: 58 - protein, 510 aa, synthetic construct
  REGION 1..510: note = Recombinant HN protein
MYSMQLASCVTLTLVLLVNSQGSGYIPEAPRDGQAYVRKDGEWVLLSTFLGSGGGGGGNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTIT

SEQ ID NO: 59 - protein, 496 aa, synthetic construct
  REGION 1..496: note = Recombinant H protein
MYSMQLASCVTLTLVLLVNSQGSGYIPEAPRDGQAYVRKDGEWVLLSTFLGSGGGGGGFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPADMQSWVPLSTDDPVIDRLYLSSHRGVIADNQAKWAVPTTRTDDKLRMETCFQQACKGKIQTLCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGEDCHAPTYLPAEVDGDVKLSSNLVILPGQDLQYVLATYDTSRVEHAVVYYVYSPGRSFSYFYPFRLPIKGVPIELQVECFTWDQKLWCRHFCVLADSESGGHITHSGMVGMGVSCTVTREDGTNRR

SEQ ID NO: 60 - protein, 459 aa, synthetic construct
  REGION 1..459: note = Recombinant H protein
MYSMQLASCVTLTLVLLVNSQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPADMQSWVPLSTDDPVIDRLYLSSHRGVIADNQAKWAVPTTRTDDKLRMETCFQQACKGKIQTLCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGEDCHAPTYLPAEVDGDVKLSSNLVILPGQDLQYVLATYDTSRVEHAVVYYVYSPGRSFSYFYPFRLPIKGVPIELQVECFTWDQKLWCRHFCVLADSESGGHITHSGMVGMGVSCTVTREDGTNRR

SEQ ID NO: 61 - protein, 497 aa, synthetic construct
  REGION 1..497: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSGSGGGSAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIGGYIPEAPRDGQAYVRKDGEWVLLSTFL

SEQ ID NO: 62 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQCVASSRRHGGGAGVCLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 63 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAIRQAGQEMILAFQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 64 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQCVASSRRHGGGAGVCLAGAALGVATAAQITAGIALHQSMLNSQAIDNLRASLETTNQAIEAIRQAGQEMILAFQGVQDYINNELIPSMNQLPCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 65 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQCVASSRRHGGGAGVCLAGAALGVATAAQITAGIALHQSMLNSQAIDNLRASLETTNQAIEAICQAGQECILAFQGVQDYINNELIPSMNQLPCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 66 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNICPVQSVASSRRHGGGAGVCLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 67 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNICPVQSVASSRRHGGGAGVCLAGAALGVATAAQITAGIALHQSMLNSQAIDNLRASLETTNQAIEAIRQAGQEMILAFQGVQDYINNELIPSMNQLPCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 68 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNICPVQSVASSRRHGGGAGVCLAGAALGVATAAQITAGIALHQSMLNSQAIDNLRASLETTNQAIEAICQAGQECILAFQGVQDYINNELIPSMNQLPCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 69 - protein, 520 aa, synthetic construct
  REGION 1..520: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNICPVQSVASSRRHGGGAGVCLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSAIEDKIEEILSKIYHIENEIARIKKLIGEAP

SEQ ID NO: 70 - protein, 520 aa, synthetic construct
  REGION 1..520: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNICPVQSVASSRRHGGGAGVCLAGAALGVATAAQITAGIALHQSMLNSQAIDNLRASLETTNQAIEAIRQAGQEMILAFQGVQDYINNELIPSMNQLPCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSAIEDKIEEILSKIYHIENEIARIKKLIGEAP

SEQ ID NO: 71 - protein, 520 aa, synthetic construct
  REGION 1..520: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNICPVQSVASSRRHGGGAGVCLAGAALGVATAAQITAGIALHQSMLNSQAIDNLRASLETTNQAIEAICQAGQECILAFQGVQDYINNELIPSMNQLPCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSAIEDKIEEILSKIYHIENEIARIKKLIGEAP

SEQ ID NO: 72 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRCVQSVASSRRHGGGAGVCLAGAALGVATAAQITAGIALHQSMLNSQAIDNLRASLETTNQAIEAICQAGQECILAFQGVQDYINNELIPSMNQLPCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 73 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPCQSVASSRRHGGGAGVCLAGAALGVATAAQITAGIALHQSMLNSQAIDNLRASLETTNQAIEAICQAGQECILAFQGVQDYINNELIPSMNQLPCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 74 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVCSVASSRRHGGGAGVCLAGAALGVATAAQITAGIALHQSMLNSQAIDNLRASLETTNQAIEAICQAGQECILAFQGVQDYINNELIPSMNQLPCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 75 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSCLNSQAIDNLRASLETTNQAIEAICQAGQECILAVQGVQDYINNELIPSMNQLSCDLIGQKLGLKLLRYYTEILSLFGPSLRDPISAEISIQALSYALGGDINKVLEKLGYSGGDLLGILESRGIKARICHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 76 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAICQAGQECILAVQGVQDYINNELIPSMNQLSCDLIGQKLGLKLLRYYTCILSLFGPSLRDPISAEISIQALSCALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 77 - protein, 513 aa, synthetic construct
  REGION 1..513: note = Recombinant F protein
MYSMQLASCVTLTLVLLVNSQIHWGNLSKIGVVGIGSASYKVMTCSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAICQAGQECILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSICYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 78 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNICPVQSVASSRRHKRFAGVCLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 79 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNICPVQSVASSRRHKRFAGVCLAGAALGVATAAQITAGIALHQSMLNSQAIDNLRASLETTNQAIEAIRQAGQEMILAFQGVQDYINNELIPSMNQLPCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 80 - protein, 513 aa, synthetic construct
  REGION 1..513: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNICPVQSVASSRRHKRFAGVCLAGAALGVATAAQITAGIALHQSMLNSQAIDNLRASLETTNQAIEAICQAGQECILAFQGVQDYINNELIPSMNQLPCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAP

SEQ ID NO: 81 - protein, 988 aa, synthetic construct
  REGION 1..988: note = Recombinant F protein
MYSMQLASCVTLTLVLLVNSQIHWGNLSKIGVVGIGSASYKVMTRSSHQSLVIKLMPNITLLNNCTRVEIAEYRRLLRTVLEPIRDALNAVTQNIRPVQSVASSRRHGGGAGVVLAGAALGVATAAQITAGIALHQSMLNSQAIDNLRASLETTNQAIEAICQAGQECILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILSAIEDKIEEILSKIYHIENEIARIKKLIGEAPGSGYIPEAPRDGQAYVRKDGEWVLLSTFLGSGGGGGGFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPADMQSWVPLSTDDPVIDRLYLSSHRGVIADNQAKWAVPTTRTDDKLRMETCFQQACKGKIQTLCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGEDCHAPTYLPAEVDGDVKLSSNLVILPGQDLQYVLATYDTSRVEHAVVYYVYSPGRSFSYFYPFRLPIKGVPIELQVECFTWDQKLWCRHFCVLADSESGGHITHSGMVGMGVSCTVTREDGTNRR

SEQ ID NO: 82 - protein, 950 aa, synthetic construct
  REGION 1..950: note = Recombinant HN/H protein
MYSMQLASCVTLTLVLLVNSQNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTITGGGSGYIPEAPRDGQAYVRKDGEWVLLSTFLGSGGGGGGFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 83 - protein, 985 aa, synthetic construct
  REGION 1..985: note = Recombinant HN/H protein
MYSMQLASCVTLTLVLLVNSQNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTITGGKLMKQIEDKIEEILSKIYHIENEIARIKKLIGEAPGSGYIPEAPRDGQAYVRKDGEWVLLSTFLGSGGGGGGFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 84 - protein, 948 aa, synthetic construct
  REGION 1..948: note = Recombinant HN/H protein
MYSMQLASCVTLTLVLLVNSQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRRGSGYIPEAPRDGQAYVRKDGEWVLLSTFLGSGGGGGGNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTIT

SEQ ID NO: 85 - protein, 981 aa, synthetic construct
  REGION 1..981: note = Recombinant HN/H protein
MYSMQLASCVTLTLVLLVNSQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRRMKQIEDKIEEILSKIYHIENEIARIKKLIGEAPGSGYIPEAPRDGQAYVRKDGEWVLLSTFLGSGGGGGGNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTIT

SEQ ID NO: 86 - protein, 580 aa, synthetic construct
  REGION 1..580: note = Recombinant H protein
MYSMQLASCVTLTLVLLVNSQRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 87 - protein, 577 aa, synthetic construct
  REGION 1..577: note = Recombinant H protein
MYSMQLASCVTLTLVLLVNSQRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 88 - protein, 579 aa, synthetic construct
  REGION 1..579: note = Recombinant H protein
MYSMQLASCVTLTLVLLVNSQLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 89 - protein, 572 aa, synthetic construct
  REGION 1..572: note = Recombinant H protein
MYSMQLASCVTLTLVLLVNSQTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 90 - protein, 550 aa, synthetic construct
  REGION 1..550: note = Recombinant HN protein
MYSMQLASCVTLTLVLLVNSQELVRMINDQGLSNQLSSITDKIRESATMIASAVGVMNQVIHGVTVSLPLQIEGNQNQLLATLATICTSQKQVSNCSTNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTIT

SEQ ID NO: 91 - protein, 543 aa, synthetic construct
  REGION 1..543: note = Recombinant HN protein
MYSMQLASCVTLTLVLLVNSQDQGLSNQLSSITDKIRESATMIASAVGVMNQVIHGVTVSLPLQIEGNQNQLLATLATICTSQKQVSNCSTNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTIT

SEQ ID NO: 92 - protein, 541 aa, synthetic construct
  REGION 1..541: note = Recombinant HN protein
MYSMQLASCVTLTLVLLVNSQGLSNQLSSITDKIRESATMIASAVGVMNQVIHGVTVSLPLQIEGNQNQLLATLATICTSQKQVSNCSTNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTIT

SEQ ID NO: 93 - protein, 549 aa, synthetic construct
  REGION 1..549: note = Recombinant HN protein
MYSMQLASCVTLTLVLLVNSQLVRMINDQGLSNQLSSITDKIRESATMIASAVGVMNQVIHGVTVSLPLQIEGNQNQLLATLATICTSQKQVSNCSTNIPLVNDLRFINGINKFIIEDYATHDFSIGHPLNMPSFIPTATSPNGCTRIPSFSLGKTHWCYTHNVINANCKDHTSSNQYVSMGILVQTASGYPMFKTLKIQYLSDGLNRKSCSIATVPDGCAMYCYVSTQLETDDYAGSSPPTQKLTLLFYNDTVTERTISPSGLEGNWATLVPGVGSGIYFENKLIFPAYGGVLPNSTLGVKSAREFFRPVNPYNPCSGPQQDLDQRALRSYFPSYFSNRRIQSAFLVCAWNQILVTNCELVVPSSNQTMMGAEGRVLLINNRLLYYQRSTSWWPYELLYEISFTFTNSGPSSVNMSWIPIYSFTRPGSGNCSGENVCPTACVSGVYLDPWPLTPYSHQSGINRNFYFTGALLNSSTTRVNPTLYVSALNNLKVLAPYGTQGLFASYTTTTCFQDTGDASVYCVYIMELASNIVGEFQILPVLTRLTIT

SEQ ID NO: 94 - DNA, 1653 bp, Measles virus
  source 1..1653: mol_type = other DNA
atgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgaggcaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagcttacgggaccccatatctgcggagatatctatccaggctttgagctatgcgcttggaggagacatcaataaggtgttagaaaagctcggatacagtggaggtgatttactgggcatcttagagagcagaggaataaaggcccggataactcacgtcgacacagagtcctacttaattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtccaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatattattggagaggttggacgtagggacaaatctggggaatgcaattgctaagttggaggatgccaaggaattgttggagtcatcggaccagatattgaggagtatgaaaggtttatcgagcacttgcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctga

SEQ ID NO: 95 - DNA, 1617 bp, Mumps virus
  source 1..1617: mol_type = other DNA
atgaaggccttttcagttacttgcttgggctttgcagtcttttcgtcttctatatgtgtgaatatcaacatcttgcagcaaattggatatatcaagcaacaagtcaggcaactaagctattactcacaaagttctagctcctacatagtggtcaagcttttaccgaatatccaacccactgataacagctgtgaatttaagagtgtaactcaatacaataagaccttgagtaacttgcttcttcccattgcagaaaacataaacaatattgcatcgccctcacctgggtcaagacgtcataaaaggtttgctggcattgccattggcattgctgcgctcggtgttgcgaccgcagcacaggtaactgccgctgtctcattagttcaagcacagacaaatgcacgtgcaatagcggcgatgaaaaattcaatacaggcaactaatcgggcaatcttcgaagtgaaggaaggcacccaacagttagctatagcggtacaagcaatacaagaccacatcaatactattatgaacacccaattgaacaatatgtcttgtcagatccttgataaccagcttgcaacctacctaggattatacctaacagaattaacaacagtgtttcagccacaattaattaatccggcattgtcaccgattagtatacaagccttgaggtctttgcttggaagtatgacgcctgcagtggttcaagcaacattatctacgtcaatttctgctgctgaaatactaagtgccggtctaatggagggtcagattgtttctgttctgctagatgagatgcagatgatagtcaagataaatattccaaccattgtcacacaatcaaatgcattggtgattgacttctactcaatttcgagctttattaataatcaagaatccataattcaattgccagacaggatcctagagatcgggaatgaacaatggagctatccagctaaaaattgtaagttgacaagacaccacatattctgccaatacaatgaggcagagaggctgagcctagaatcaaaactatgccttgcaggtaatataagtgcctgtgtgttctcacccatagcagggagttatatgaggcgatttgtagcactggatggaacaattgttgcaaactgtcgaagtctaacgtgtctatgcaagagtccatcttatcctatataccaacctgaccatcatgcagtcacgaccattgatctaaccacatgtcaaacattgtccctagacggattggacttcagcattgtctctctaagcaacatcacttacgctgagaaccttaccatttcattgtctcaaacaatcaatactcaacccattgacatatcaactgaactgagtaaagttaatgcatccctccaaaatgccgttaagtacataaaggagagcaaccatcaactccaatctgtgagtgtaaattccaaaattggagctataattgtagcagccttagttttgagcatcctgtcaattatcatttcgctattgttttgctgctgggcttacattgcaactaaagaaatcagaagaatcaacttcaaaacaaatcatatcaacacaatatcgagtagtgtcgatgatctcatcaggtactaa

SEQ ID NO: 96 - DNA, 1854 bp, Measles virus
  source 1..1854: mol_type = other DNA
atgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattcatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatag

SEQ ID NO: 97 - DNA, 1749 bp, Mumps virus
  source 1..1749: mol_type = other DNA
atggagccctcgaaattcttcacaatatcggacagtgccacctttgcacctgggcctgtcagcaatgcggctgacaagaagacattccgaacctgcttccgaatactggtactatctgtacaagctgtcaccctcatattggttattgtcactttaggtgagcttgtaaggatgatcaatgatcaaggcttgagcaatcagttgtcttcaattacagacaagataagagagtcagctactatgattgcatctgctgtgggagtaatgaatcaagttattcatggagtaacggtatccttacccctacaaattgagggaaaccaaaatcaattgttagccacacttgccacaatctgcgccagccaaaaacaagtctcaaactgctctacaaacatccccttagtcaatgacctcaggtttataaatgggatcaataaatttattattgaagattacgcaactcatgatttctctatcggccatccactcaatatgcccagctttatcccaactgcaacttcacccaatggttgcacaagaattccatccttttctttaggtaagacacactggtgctacacacataatgtaattaatgccaactgcaaggaccatacttcgtctaaccaatatgtgtccatggggattctcgttcagaccgcgtcagggtatcctatgttcaaaaccttaaaaatccaatatctcagtgatggcctgaatcggaaaagctgctcaattgcaacagtccctgatgggtgcgcgatgtactgttatgtctctactcaacttgaaaccgacgactatgcggggtccagtccacccacccaaaaacttaccctgttattctataatgacaccgtcacagaaaggacaatatctccatctggtcttgaagggaattgggctactttggtgccaggagtggggagtgggatatattttgagaataagttgatcttccctgcatatgggggtgtcttgcccaatagtacactcggggttaaatcagcaagagaattttttcggcctgttaatccatataatccatgttcaggaccacaacaagatttagatcagcgtgctttgaggtcatacttcccaagttacttctctaatcgaagaatacagagtgcatttcttgtctgtgcctggaatcagatcctagttacaaattgtgagctagttgtcccctcaagcaatcagacaatgatgggtgcagaagggagagttttattgatcaataatcgactattatattatcagagaagtaccagctggtggccgtatgaactcctctacgagatatcattcacatttacaaactctggtccatcatctgtaaatatgtcctggatacctatatattcattcactcgtcctggttcaggcaattgcagtggtgaaaatgtgtgcccgactgcttgtgtgtcaggggtttatcttgatccctggccattaactccatatagccaccaatcaggtattaacagaaatttctatttcacaggtgcactattaaattcaagtacaactagagtaaatcctaccctttatgtctctgcccttaataatcttaaagtactagccccatatggtactcaaggactgtttgcctcgtacaccacaaccacctgctttcaagataccggtgatgctagtgtgtattgtgtttatattatggagctagcatcaaatattgttggagaattccaaattctacctgtgctaactagattgactatcacttga

SEQ ID NO: 98 - protein, 476 aa, Mumps virus
MKAYPVICLGFAVFSSSICVNINILQQIGYIKQQVRQLSYYSQSSSSYVVVKLLPNIQPTDNSCEFKSVTQYNKTLSNLLLPIAENINNIASPSPGSRRHKRFAGIAIGIAALGVATAAQVTAAVSLVQAQTNARAIAAMKNSIQATNRAIFEVKEGTQQLAIAVQAIQDHINTIMNTQLNNMSCQILDNQLATYLGLYLTELTTVFQPQLINPALSPISIQALRSLLGSMTPAVVQATLSTSISAAEILSAGLMEGQIVSVLLDEMQMIVKINIPTIVTQSNALVIDFYSISSFINNQESIIQLPDRILEIGNEQWSYPAKNCKSTRHHIFCQYNEAERLSLETKLCLAGNISACVFSPIAGSYMRRFVALDGTIVANCRSLTCLCKSPSYPIYQPDHHAVTTIDLTSCQTLSLDGLDFSIVSLSNITYAENLTISLSQTINTQPIDISTELSKVNASLQNAVKYIKESNHQLQS

SEQ ID NO: 99 - protein, 55 aa, synthetic construct
  REGION 1..55: note = transmembrane domain and cytoplasmic tail
GAIIVAALVLSILSIIISLLFCCWAYIATKEIRRINFKTNHINTISSSVDDLIRY

SEQ ID NO: 100 - protein, 17 aa, synthetic construct
  REGION 1..17: note = transmembrane domain
CIVYILIAVCLGGLIGI

SEQ ID NO: 101 - protein, 57 aa, synthetic construct
  REGION 1..57: note = transmembrane domain and cytoplasmic tail
CIVYILIAVCLGGLIGIPALICCCRGRCNKKGEQVGMSRPGLKPDLTGTSKSYVRSL

SEQ ID NO: 102 - protein, 476 aa, Mumps virus
MKAFLVTCLGFAVFSSSICVNINILQQIGYIKQQVRQLSYYSQSSSSYIVVKLLPNIQPTDNSCEFKSVTQYNKTLSNLLLPIAENINNIASPSPGSRRHKRFAGIAIGIAALGVATAAQVTAAVSLVQAQTNARAIAAMKNSIQATNRAIFEVKEGTQQLAIAVQAIQDHINTIMNTQLNNMSCQILDNQLATYLGLYLTELTTVFQPQLINPALSPISIQALRSLLGSMTPAVVQATLSTSISAAEILSAGLMEGQIVSVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTTCQTLSLDGLDFSIVSLSNITYAENLTISLSQTINTQPIDISTELSKVNASLQNAVKYIKESNHQLQS

SEQ ID NO: 103 - protein, 476 aa, Mumps virus
MKAFSVTCLSFAVFSSSICVNINILQQIGYIKQQVRQLSYYSQSSSSYIVVKLLPNIQPTENSCEFKSITQYNKTLSNLLLPIAENINNIASPSPGSRRHKRFAGIAIGIAALGVATAAQVTAAVSLVQAQTNARAIAAMKNSIQATNRAIFEVKEGTQQLAIAVQALQDHINTIMNTQLNNMSCQILDNQLATYLGLYLTELTTVFQPQLINPALSPISIQALRSLLGSMTPAVVQATLSTSISAAEILSAGLMEGQIVSVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTIDLTTCQTLSLDGLDFSIVSLSNITYAENLTISLSQTINTQPIDISTELSKVNASLQNAVKYIKESNHQLQS

SEQ ID NO: 104 - protein, 476 aa, Mumps virus
MKAFLVICLGFAVFSSSICVNINILQQVGYIKQQVRQLSYYSQSSSSYIVVKLLPNIQPTDNSCEFKSVTQYNKTLGNLLLPIAENINNIASPSPGSRRHKRFAGIAIGIAALGVATAAQVTAAVSLVQAQTNARAIAAMKNSIQATNRAIFEVKEGTQQLAIAVQAIQDHINTIMNTQLSNMSCQILDNQLATYLGLYLTELTTVFQPQLINPALSPISIQALRSLLGSMTPAVVQATLSTSISAAEILSAGLMEGQIVSVLLDEMQMIVKINIPTIVTQSNALVIDFYSISSFINNQESIIQLPDRILEIGNEQWSYPAKNCKLTRHNIFCQYNEAERLSLESKLCLAGNISACVFSPIAGSYMRRFVALDGTIVANCRSLTCLCKSPSYPIYQPDHHAVTTIDLTTCQTLSLDGLDFSIVSLSNITYAENLTISLSQTINTQPIDISTELSKVNASLQNAVKYIKESNQQLQS

SEQ ID NO: 105 - protein, 476 aa, Mumps virus
MKASLVTCLGFAVFSSSICVNINILQQIGYIKQQVRQLSYYSQSSSSYIVVKLLPNIQPTDNSCEFKSVTQYNKTLSNLLLPIAENINNIASPSPGSRRHKRFAGIAIGIAALGVATAAQVTAAVSLVQAQTNARAIAAMKNSIQATNRAIFEVKEGTQQLAIAVQAIQNHINTIMNTQLNNMSCQILDNQLATYLGLYLTELTTVFQPQLINPALSPISIQALRSLLGSMTPAVVQATLSTSISAAEILSAGLMEGQIVSVLLDEMQMIVKINIPTIVTQSNALVIDFYSISSFINNQESIIQLPDRILEIGNEQWSYPAKNCKLTRHNIFCQYNEAERLSLESKLCLAGNISACVFSPIAGSYMRRFVALDGTIVANCRSLTCLCKSPSYPIYQPDHHAVTTIDLTTCQMLSLDGLDFSIVSLSNITYAENLTISLSQTINTQPIDISTELIKVNASLQNAVKYIKESNHQLQS

SEQ ID NO: 106 - protein, 476 aa, Mumps virus
MKAFSVTCLGFAVFSSSICVNINILQQIGYIKQQVRQLSYYSQSSSSYIVVKLLPNIQPPDDSCEFKSVISYNKTLSNLLLPIAENINNIASPSPGSRRHKRFAGIAIGIAALGVATAAQVTAAVSLVQAQTNARAIAAMKNSIQATNRAIFEVKEGTQQLAIAVQAIQDHINTIMNTQLNNMSCQILDNQLATYLGLYLTELTTVFQPQLINPALSPISIQALRSLLGSMTPAVVQAALSTSISAAEILSAGLMEGQIVSVLLDEMQMIVKINIPTIVTQSNALVIDFYSISSFINNQESIIQLPDRILEIGNEQWSYPAKNCKLTRHHIFCQYNEAERLSLESKLCLAGNISACVFSPIAGSYMRRFVALDGTIVANCRSLTCLCKSPSYPIYQPDHHAVTTVDLTTCQTLSLDGLDFSIVSLSNITYAENLTISLSQTINTQPIDISTELSKVNASLQNAVKYIKESNHQLQS

(19) United States
(12) Patent Application Publication
(10) Pub. No.: US 2026/0240984 A1
(43) Pub. Date: Aug. 20, 2026

(54) MUMPS AND MEASLES VIRUS IMMUNOGENS AND THEIR USE

(71) Applicant: The United States of America, as represented by the Secretary, Department of Health and Human Ser, Bethesda, MD (US)

(72) Inventors: Barney Graham, Smyrna, GA (US); Guillaume Stewart-Jones, Cambridge, MA (US); Rebecca J. Loomis, Bethesda, MD (US)

(73) Assignee: The United States of America, as represented by the Secretary, Department of Health and Human Ser, Bethesda, MD (US)

(21) Appl. No.: 19/660,147

(22) Filed: Apr. 27, 2026

Related U.S. Application Data
(62) Division of application No. 17/784,605, filed on Dec. 11, 2020, now Pat. No. 12,636,360.
(60) Provisional application No. 62/946,902, filed on Dec. 11, 2019.

(51) Int. Cl. A61K 39/165; A61K 39/00; A61P 31/14; C07K 14/12

(52) U.S. Cl. CPC A61K 39/165; A61P 31/14; C07K 14/12; A61K 2039/575; C12N 2760/18032

(57) ABSTRACT

Embodiments of immunogens comprising a recombinant Mumps (MuV) F ectodomain trimer stabilized in a prefusion conformation or a recombinant Measles (MeV) F ectodomain trimer stabilized in a prefusion conformation are provided. Also provided are embodiments of immunogens comprising chimeric proteins comprising the recombinant MuV or MeV F ectodomain trimer and one or more MuV HN or MeV H ectodomains. Also disclosed are nucleic acids encoding the immunogens and methods of their production. Methods for inducing an immune response in a subject by administering a disclosed immunogen to the subject are also provided. In some embodiments, the immune response treats or inhibits MuV and/or MeV infection in a subject.

### CROSS-REFERENCE TO RELATED APPLICATIONS

This is the continuation of U.S. patent application Ser. No. 17/784,605, filed Jun. 10, 2022, which is the U.S. National Stage of International Application No. PCT/US2020/064619, filed Dec. 11, 2020, which was published in English under PCT Article 21(2), which in turn claims the benefit of the earlier filing date of U.S. Provisional Application No. 62/946,902, filed Dec. 11, 2019, all of which are incorporated herein by reference in their entireties.

### FIELD

This disclosure relates to polypeptides, polynucleotides, compositions, and methods of their use, for elicitation and detection of an immune response to mumps virus (MuV) and measles virus (MeV).

### INCORPORATION OF ELECTRONIC SEQUENCE LISTING

The Sequence Listing is submitted as an XML file named “Sequence.xml,” created on Apr. 22, 2026, 185,090 bytes, which is incorporated by reference herein.

### BACKGROUND

MeV and MuV are highly contagious paramyxoviruses that can be transmitted by respiratory droplets from or direct contact with an infected person. The resulting diseases can lead to serious complications or death among children. The existing vaccines for MeV and MuV are live attenuated virus, which are administered in two subcutaneous doses at 1 year of age and as early as one month later. Two doses of a combination measles, mumps and rubella vaccine are 97% effective against measles and 88% effective against mumps. A single dose of a combination measles, mumps and rubella vaccine is 93% effective against measles and 78% effective against mumps.

Despite the effectiveness of the current licensed vaccines against MeV and MuV, incidences of both have increased in recent years. Contributing factors include reduced vaccination rates due to vaccine hesitancy and circulation of divergent strains against which the licensed MMR vaccine offers limited protection.

In the case of MuV, recent studies have shown that immunity wanes significantly after the second MMR vaccination which normally occurs in childhood. Additionally, there has been a drifting of genotypes in the current circulating MuV strains away from the Jeryl-Lynn strain in the standard Mumps vaccine. In response to recent recurring MuV disease outbreaks in the U.S. and Europe, the Advisory Committee on Immunization Practices is advising a third MMR vaccination to boost protection. However, existing immunity neutralizes a third MMR vaccination limiting its effectiveness.

### SUMMARY

Disclosed herein are recombinant MuV F ectodomain trimers and recombinant MeV F ectodomain trimers comprising protomers comprising one or more modifications (such as amino acid substitutions) for stabilization in a prefusion conformation. Further provided are embodiments of the recombinant MuV F ectodomain trimers and recombinant MeV F ectodomain trimers linked to MuV HN ectodomain or a MeV H ectodomain. Chimeric proteins are provided, that include combinations of MuV F ectodomain trimer and MeV H ectodomain, or MeV F ectodomain trimer and MuV HN ectodomain. Embodiments of such proteins are demonstrated to produce a superior immune response in animal models, and can be used, for example, to elicit or boost an immune response to MeV and/or MuV in a subject.

In some embodiments, the immunogen comprises a recombinant MuV F ectodomain trimer stabilized in a prefusion conformation by one or more amino acid substitutions in protomers of the trimer, the amino acid substitutions comprising cysteine substitutions that form a non-natural disulfide bond to stabilize the MuV F ectodomain trimer in the prefusion conformation. In some embodiments, the recombinant MuV F ectodomain trimer is stabilized in the prefusion conformation by a non-natural disulfide bond between the cysteine substitutions in protomers of the trimer at MuV F positions 206 and 223. In some embodiments, the protomers of the trimer further comprise a mutation to remove a F1/F2 furin cleavage site of the MuV F ectodomain. In some embodiments, the protomers of the recombinant MuV F ectodomain trimer are fused C-terminally to a trimerization domain, such as a GCN4 trimerization domain. In additional embodiments, the protomers of the recombinant MuV F ectodomain trimer are linked to a heterologous protein, such as a MuV HN ectodomain or a MeV H ectodomain.

In some embodiments, the immunogen comprises a recombinant MeV F ectodomain trimer stabilized in a prefusion conformation by one or more amino acid substitutions in protomers of the trimer, the amino acid substitutions comprising cysteine substitutions that form a non-natural disulfide bond to stabilize the MeV F ectodomain trimer in the prefusion conformation. In some embodiments, the recombinant MeV F ectodomain trimer is stabilized in the prefusion conformation by a non-natural disulfide bond between the cysteine substitutions in protomers of the trimer at MeV F positions 165 and 171. In some embodiments, the protomers of the trimer further comprise a mutation to remove a F1/F2 furin cleavage site of the MeV F ectodomain. In some embodiments, the protomers of the recombinant MeV F ectodomain trimer are fused C-terminally to a trimerization domain, such as a GCN4 trimerization domain. In additional embodiments, the protomers of the recombinant MeV F ectodomain trimer are linked to a heterologous protein, such as a MuV HN ectodomain or a MeV H ectodomain.

In some embodiments, an immunogen is provided that comprises a trimer of fusion proteins, each fusion protein comprising, in an N- to C-terminal direction: a trimerization domain and one or more MuV HN ectodomains or MeV H ectodomain.

In some embodiments, the immunogen comprises a dimer of a MeV H ectodomain head.

Nucleic acid molecules encoding the disclosed proteins are also provided, as are vectors including the nucleic acid molecules, and methods of their production.

Immunogenic compositions including a disclosed immunogen that are suitable for administration to a subject are also provided, and may also be contained in a unit dosage form. The immunogen may also contain a carrier to facilitate presentation to the immune system.

Methods of inducing an immune response in a subject are disclosed, as are methods of inhibiting or preventing a MuV or MeV infection in a subject, by administering to the subject an effective amount of a disclosed immunogen, nucleic acid molecule, or vector.

The foregoing and other features and advantages of this disclosure will become more apparent from the following detailed description of several embodiments which proceeds with reference to the accompanying figures.

### BRIEF DESCRIPTION OF THE FIGURES

FIGS. 1A-1E. Structure-based design of a disulfide stabilized prefusion mumps F trimer. (FIG. 1A) Structure-based design of prefusion mumps F glycoprotein trimer by systematic screening of disulfides and GCN4 attachment positions, showing both yield and percentage of prefusion relative to postfusion F conformation as determined by negative-stain EM. (FIG. 1B) Homogenous prefusion F trimers were observed at high yield with combination of V206C/A223C with 476-GCN4, upper panel and postfusion is shown in the lower panel. (FIG. 1C) S200 gel filtration analysis shows monodispersal of prefusion F (PreF), glycosylated and deglycosylated, HN and prefusion F-HN. (FIG. 1D) Crystal structure to 2.16 Å resolution of prefusion mumps F trimer (SEQ ID NO: 11) with protomers shown, where residues that undergo >5 Å conformational change to transition to the postfusion conformation shown in black. GCN4 trimerization (TD) motif is shown in dotted line, connected to F residue 476. Zoom insets highlight mutated residues to stabilize the trimeric prefusion F structure. (FIG. 1E) A single protomer of the prefusion mumps trimer showing the D1-3 subregions and the locations of the six N-linked glycans per protomer.

FIGS. 2A-2H. Structural comparison of mumps and PIV5 prefusion Fs and analysis of mumps F and HN genotypic variation. (FIG. 2A) Structural superimposition of mumps prefusion F trimer and the related paramyxovirus PIV5 prefusion F trimer showing similar overall topology (RMSD=1.68 Å), yet share 49% sequence identity. (FIG. 2B) The apical loops of the mumps preF associate in a ‘closed cap’ assembly, whereas in PIV5, these loops are splayed apart (top panel) and the residues that stabilize the mumps preF apex are T178, T179 and N181 (lower panel). (FIG. 2C) The mumps F glycoprotein is glycosylated with 6 glycans per protomer totaling 18 per trimer with 6 glycans in the conformationally mobile region (black) and 12 glycans in the less conformationally variable region, providing substantial glycan shielding to the trimer. (FIG. 2D) Phylogenetic analysis of fusion glycoproteins from mumps genotypes A-J. (FIG. 2E) Phylogenetic analysis of hemagglutinin-neuraminidase glycoproteins from mumps genotypes A-J. (FIGS. 2F and 2G) Sequence alignment of Jeryl Lynn vaccine strain (genotype A, SEQ ID NO: 98) with fusion glycoproteins from mumps genotypes C (SEQ ID NO: 102), D (SEQ ID NO: 103), F (SEQ ID NO: 104), G (SEQ ID NO: 105), and H (SEQ ID NO: 106). (FIG. 2H) Structural mapping of mumps genotype variations on prefusion F (left panels) and HN (right panels), showing A-J genotype variation (top panels) and genotype G versus A (Jeryl Lynn (JL)) (lower panels), showing residue differences and glycan differences.

FIGS. 3A and 3B. Design and immunization scheme for mumps F and preF-HN immunogen variants. (FIG. 3A) Immunization in CB6F1/J mice was performed 3 times at 10 g/dose with Poly(I:C) adjuvant at times 0, 3 and 10 weeks with blood sampling at 2, 5, 12 and 16 weeks. (FIG. 3B) Negative-stain characterization of mumps F and preF-HN proteins confirming the conformational and molecular identity of the immunogens and mumps neutralization titers for postfusion F (left), prefusion F (center), and prefusion F-HN (right) immunogens elicited after 1, 2 or 3 immunizations in mice (shown in arrows), and PRN titers for genotype G virus, Jeryl Lynn genotype A virus, and genotype H virus.

FIGS. 4A-4E. Design of prefusion mumps F-HN immunogen, analysis of sera for specificity and durability of neutralization titers for three diverse mumps genotype viruses. (FIG. 4A) Negative-stain characterization of mumps preF-HN fusion protein, showing positions of preF head, GCN4 trimerization domain and three HN headgroups. (FIG. 4B) Binding titers for immunized mice using the mumps prefusion F probe and (FIG. 4C) HN probe using Octet Bioinferometry. (FIG. 4D) Durability analysis of mice immunized with three doses of 10 g prefusion F protein with Poly(I:C) with monthly serum sampling and PRNT analyses for six months against genotype G virus, Jeryl Lynn virus, and genotype H virus. (FIG. 4E) Durability analysis of mice immunized with three doses of 10 g prefusion F-HN protein with Poly(I:C) with monthly serum sampling and PRNT analyses for six months against genotype G virus, Jeryl Lynn virus and genotype H virus.

FIGS. 5A-5C. Structure-Based Design of prefusion Measles F. (FIG. 5A) Structure-based design of prefusion-stabilized measles F glycoprotein mutations that stabilize the prefusion conformation. (FIG. 5B) Negative stain EM analysis of measles F variants including the indicated non-native disulfide bond and a C-terminal GCN4 trimerization domain, and (FIG. 5C) Immunogenicity of prefusion (MeV F R165C/M171C-486-GCN4, SEQ ID NO: 38) and postfusion (native MeV F sequence with-486-GCN4) measles F glycoproteins.

FIGS. 6A-6C. Design of mumps pre-F-measles H chimeric immunogen. (FIG. 6A) Negative-stain characterization of measles H dimer (upper left), measles H trimer (upper middle), mumps HN trimer (upper right), and mumps pre-F with measles H (MuV F 206C/223C-476+GCN4/Fd+MeV-H, SEQ ID NO: 28) (lower). (FIG. 6B) Measles plaque reduction neutralization titer assays (PRNT). (FIG. 6C) Mumps (genotype G) PRNT assays.

### STRUCTURAL COORDINATES

The atomic coordinates of the crystal structure of MuV F ectodomain trimer stabilized in a prefusion conformation are recited in Table 1 of U.S. Provisional Application No. 62/946,902, filed Dec. 11, 2019, which is incorporated by reference herein in its entirety, and which is submitted therein as an ASCII text file in the form of the file named “Table_1.txt” (~555 KB), which was created on Dec. 9, 2019.

### DETAILED DESCRIPTION

### I. Summary of Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references. As used herein, the singular forms “a,” “an,” and “the,” refer to both the singular as well as plural, unless the context indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

Adjuvant: A vehicle used to enhance antigenicity. In some embodiments, an adjuvant includes a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). In some embodiments, the adjuvant used in a disclosed immunogenic composition is a combination of lecithin and carbomer homopolymer (such as the ADJUPLEX™ adjuvant available from Advanced BioAdjuvants, LLC, see also Wegmann, Clin Vaccine Immunol, 22(9): 1004-1012, 2015). Additional adjuvants for use in the disclosed immunogenic compositions include the QS21 purified plant extract, Matrix M, ASO1, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL, immune stimulating complex (ISCOM) matrix, and toll-like receptor (TLR) agonists, such as TLR-9 agonists, Poly I:C, or PolyICLC. (See, e.g., Singh (ed.) Vaccine Adjuvants and Delivery Systems. Wiley-Interscience, 2007).

Administration: The introduction of a composition into a subject by a chosen route. Administration can be local or systemic. For example, if the chosen route is intranasal, the composition (such as a composition including a disclosed recombinant MuV F ectodomain trimer or recombinant MeV F ectodomain trimer) is administered by introducing the composition into the nasal passages of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of an amino acid in a polypeptide with one or more different amino acids. In the context of a protein sequence, an amino acid substitution is also referred to as a mutation.

Antibody: An immunoglobulin, antigen-binding fragment, or derivative thereof, that specifically binds and recognizes an analyte (antigen) such as MuV or MeV F protein, an antigenic fragment thereof, or a dimer or multimer of the antigen. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity. Non-limiting examples of antibodies include, for example, intact immunoglobulins and variants and fragments thereof that retain binding affinity for the antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2^{nd} Ed., Springer Press, 2010).

Carrier: An immunogenic molecule to which an antigen can be linked. When linked to a carrier, the antigen may become more immunogenic. Carriers are chosen to increase the immunogenicity of the antigen and/or to elicit antibodies against the carrier which are diagnostically, analytically, and/or therapeutically beneficial. Useful carriers include polymeric carriers, which can be natural (for example, proteins from bacteria or viruses), semi-synthetic or synthetic materials containing one or more functional groups to which a reactant moiety can be attached.

Conservative variants: “Conservative” amino acid substitutions are those substitutions that do not substantially affect or decrease a function of a protein, such as the ability of the protein to induce an immune response when administered to a subject. The term conservative variation also includes the use of a substituted amino acid in place of an unsubstituted parent amino acid. Furthermore, individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the recombinant MuV or MeV F ectodomain trimer, such as the ability to induce an immune response when administered to a subject.

For instance, if an amino acid residue is essential for a function of the protein, even an otherwise conservative substitution may disrupt that activity. Thus, a conservative substitution does not alter the basic function of a protein of interest.

Control: A reference standard. In some embodiments, the control is a negative control sample obtained from a healthy patient. In other embodiments, the control is a positive control sample obtained from a patient diagnosed with MuV or MeV infection. In still other embodiments, the control is a historical control or standard reference value or range of values (such as a previously tested control sample, such as a group of MuV or MeV patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Degenerate variant: In the context of the present disclosure, a “degenerate variant” refers to a polynucleotide encoding a polypeptide that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Effective amount: An amount of agent, such as an immunogen, that is sufficient to elicit a desired response, such as an immune response in a subject. It is understood that to obtain a protective immune response against an antigen of interest can require multiple administrations of a disclosed immunogen, and/or administration of a disclosed immunogen as the “prime” in a prime boost protocol wherein the boost immunogen can be different from the prime immunogen. Accordingly, an effective amount of a disclosed immunogen can be the amount of the immunogen sufficient to elicit a priming immune response in a subject that can be subsequently boosted with the same or a different immunogen to elicit a protective immune response.

In one example, a desired response is to inhibit or reduce or prevent MuV infection. The MuV infection does not need to be completely eliminated or reduced or prevented for the method to be effective. For example, administration of an effective amount of the agent can decrease the MuV infection (for example, as measured by infection of cells, or by number or percentage of subjects infected by MuV) by a desired amount, for example by at least 50%, at least 60%, at least 70%, at least 80%, at least 90%, at least 95%, at least 98%, or even at least 100% (elimination or prevention of detectable MuV infection), as compared to a suitable control.

In one example, a desired response is to inhibit or reduce or prevent MeV infection. The MeV infection does not need to be completely eliminated or reduced or prevented for the method to be effective. For example, administration of an effective amount of the agent can decrease the MeV infection (for example, as measured by infection of cells, or by number or percentage of subjects infected by MeV) by a desired amount, for example by at least 50%, at least 60%, at least 70%, at least 80%, at least 90%, at least 95%, at least 98%, or even at least 100% (elimination or prevention of detectable MeV infection), as compared to a suitable control.

In one example, a desired response is to inhibit or reduce or prevent both MuV and MeV infection. The MuV and MeV infections do not need to be completely eliminated or reduced or prevented for the method to be effective. For example, administration of an effective amount of the agent can decrease the MuV and MeV infections (for example, as measured by infection of cells, or by number or percentage of subjects infected by MuV and/or MeV) by a desired amount, for example by at least 50%, at least 60%, at least 70%, at least 80%, at least 90%, at least 95%, at least 98%, or even at least 100% (elimination or prevention of detectable MuV and/or MeV infection), as compared to a suitable control.

Expression: Transcription or translation of a nucleic acid sequence. For example, a gene is expressed when its DNA is transcribed into an RNA or RNA fragment, which in some examples is processed to become mRNA. A gene may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. The term “expression” is used herein to denote either transcription or translation. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

GCN4 trimerization domain: A trimerization domain from the GCN4 protein that comprises a leucine zipper amino acid sequence that naturally forms a trimeric structure. Embodiments of the GCN4 trimerization domain is described, for example, Harbury et al. (1993 Science 262:1401-1407). In some examples, a GCN4 trimerization domain can be included in the amino acid sequence of a disclosed recombinant protein so that the recombinant protein will trimerize. A non-limiting example of a GCN4 trimerization domain sequence for use with the disclosed embodiments is provided as IEDKIEEILSKIYHIENEIARIKKLIGEAP (SEQ ID NO: 33).

Heterologous: Originating from a different genetic source.

Host cells: Cells in which a vector can be propagated and its nucleic acid expressed. The cell may be prokaryotic or eukaryotic. The term also includes any progeny of the subject host cell. It is understood that all progeny may not be identical to the parental cell since there may be mutations that occur during replication.

However, such progeny are included when the term “host cell” is used.

Immune response: A response of a cell of the immune system, such as a B cell, T cell, or monocyte, to a stimulus. In one embodiment, the response is specific for a particular antigen (an “antigen-specific response”).

In one embodiment, an immune response is a T cell response, such as a CD4+ response or a CD8+ response. In another embodiment, the response is a B cell response, and results in the production of specific antibodies.

Immunogen: A compound, composition, or substance (for example, a recombinant MuV or MeV F ectodomain trimer) that can elicit an immune response in an animal, including compositions that are injected or absorbed into an animal. Administration of an immunogen to a subject can lead to protective immunity against a pathogen of interest.

Immunogenic composition: A composition comprising a disclosed immunogen that induces a measurable CTL response against MuV or MeV, or induces a measurable B cell response (such as production of antibodies) against the MuV or MeV, when administered to a subject. It further refers to isolated nucleic acid molecules and vectors encoding a protomer of a disclosed recombinant MuV or MeV F ectodomain trimer that can be used to express the protomer (and thus be used to elicit an immune response against recombinant MuV or MeV F ectodomain trimer). For in vivo use, the immunogenic composition will typically include the recombinant MuV or MeV F ectodomain trimer or a nucleic acid molecule encoding a protomer of the recombinant MuV or MeV F ectodomain trimer in a pharmaceutically acceptable carrier and may also include other agents, such as an adjuvant.

Inhibiting or treating a disease: Inhibiting the full development of a disease or condition, for example, in a subject who is at risk for a disease such as MuV infection or MeV infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Linker and Linked: A bi-functional molecule that can be used to link two molecules into one contiguous molecule. Non-limiting examples of peptide linkers include glycine-serine peptide linkers. Unless context indicates otherwise, reference to “linking” a first polypeptide and a second polypeptide, or to two polypeptides “linked” together, or to a first polypeptide having a “linkage” to a second polypeptide, refers to covalent linkage by peptide bond (for example via a peptide linker) such that the first and second polypeptides form a contiguous polypeptide chain. If a peptide linker is involved, the covalent linkage of the first and second polypeptides can be to the N- and C-termini of the peptide linker. Typically, such linkage is accomplished using molecular biology techniques to genetically manipulate DNA encoding the first polypeptide linked to the second polypeptide by the peptide linker.

Native protein, sequence, or disulfide bond: A polypeptide, sequence or disulfide bond that has not been modified, for example, by selective mutation. For example, selective mutation to focus the antigenicity of the antigen to a target epitope, or to introduce a disulfide bond into a protein that does not occur in the native protein. Native protein or native sequence are also referred to as wild-type protein or wild-type sequence. A non-native disulfide bond is a disulfide bond that is not present in a native protein, for example, a disulfide bond that forms in a protein due to introduction of one or more cysteine residues into the protein by genetic engineering.

Measles: An infectious disease caused by measles virus. Symptoms usually develop 10-12 days after exposure to an infected person and last 7-10 days. Initial symptoms typically include fever, cough, runny nose, and inflamed eyes. Small white spots known as Koplik's spots may form inside the mouth two or three days after the start of symptoms. A red, flat rash which usually starts on the face and then spreads to the rest of the body typically begins three to five days after the start of symptoms. Common complications include diarrhea, middle ear infection, and pneumonia. These occur in part due to measles-induced immunosuppression. Less commonly seizures, blindness, or inflammation of the brain may occur.

Measles virus: A non-segmented, negative-stranded RNA virus of the family Paramyxoviridae, genus Morbillivirus that causes measles disease. Measles virus genomic RNA contains 6 linked transcription units that encode open reading frames for eight proteins: the nucleoprotein (N), phosphoprotein (P), C protein, V protein, matrix (M) protein, fusion (F) protein, hemagglutinin (H) protein, and the large (L) protein. There are at least 7 known genotypes of MeV, designated as genotypes A, B, C, D, F, G, and H, that are currently circulating globally.

MeV fusion (F) protein: An envelope glycoprotein of MeV that facilitates fusion of viral and cellular membranes. In nature, the F protein from MeV is initially synthesized as a single polypeptide precursor approximately 550 amino acids in length, designated F₀. F₀ includes an N-terminal signal peptide that directs localization to the endoplasmic reticulum, where the signal peptide is proteolytically cleaved. The remaining F₀ residues oligomerize to form a trimer and may be proteolytically processed by a cellular protease to generate two disulfide-linked fragments, F₁ and F₂. In MeV F the cleavage site is located approximately between residues 113/114. The smaller of these fragments, F₂, originates from the N-terminal portion of the F₀ precursor (approximately residues 24-113). The larger of these fragments, F₁, includes the C-terminal portion of the F₀ precursor (approximately residues 114-550) including an extracellular/luminal region (approximately residues 110-486), and a transmembrane and cytosolic regions (approximately residues 487-550). The extracellular portion of the MeV F protein is the MeV F ectodomain, which includes the F₂ protein and the F₁ ectodomain.

The MeV F protein exhibits remarkable sequence conservation within MeV strains. In view of this conservation, the person of ordinary skill in the art can easily compare amino acid positions of different MeV F proteins. Unless context indicates otherwise, the numbering of MeV F amino acids is made with reference to SEQ ID NO: 36 (NCBI Reference Sequence P35973.1, which is incorporated by reference herein):

mglkvnvsaifmavlltlqtptgQIHWGNLSKIGVVGIGSASYKVMTRS
SHQSLVIKLMPNITLLNNCTRVEIAEYRRLLRTVLEPIRDALNAMTQNI
RPVQSVASSRRHKRFAGVVLAGAALGVATAAQITAGIALHQSMLNSQAI
DNLRASLETTNQAIEAIRQAGQEMILAVQGVQDYINNELIPSMNQLSCD
LIGQKLGLKLLRYYTEILSLFGPSLRDPISAEISIQALSYALGGDINKV
LEKLGYSGGDLLGILESRGIKARITHVDTESYLIVLSIAYPTLSEIKGV
IVHRLEGVSYNIGSQEWYTTVPKYVATQGYLISNFDESSCTFMPEGTVC
SQNALYPMSPLLQECLRGSTKSCARTLVSGSFGNRFILSQGNLIANCAS
ILCKCYTTGTIINQDPDKILTYIAADHCPVVEVNGVTIQVGSRRYPDAV
YLHRIDLGPPILLERLDVGTNLGNAIAKLEDAKELLESSDQILRSMKGL
SSTCIVYILIAVCLGGLIGIPALICCCRGRCNKKGEQVGMSRPGLKPDL
TGTSKSYVRSL

Three MeV F protomers oligomerize in the mature F protein, which adopts a metastable prefusion conformation that is triggered to undergo a conformational change to a postfusion conformation upon contact with a target cell membrane. This conformational change exposes a hydrophobic sequence, known as the fusion peptide, which is located at the N-terminus of the F₁ ectodomain, and which associates with the host cell membrane and promotes fusion of the membrane of the virus, or an infected cell, with the target cell membrane.

An MeV F ectodomain trimer “stabilized in a prefusion conformation” comprises one or more amino acid substitutions, deletions, or insertions compared to a corresponding native MeV F sequence that provide for increased retention of the prefusion conformation compared to MeV F ectodomain trimers formed from a corresponding native MeV F sequence. The “stabilization” of the prefusion conformation can be, for example, energetic stabilization (for example, reducing the energy of the prefusion conformation relative to the postfusion open conformation) and/or kinetic stabilization (for example, reducing the rate of transition from the prefusion conformation to the postfusion conformation). Additionally, stabilization of the MeV F ectodomain trimer in the prefusion conformation can include an increase in resistance to denaturation compared to a corresponding native MeV F sequence. Methods of determining if a MeV F ectodomain trimer is in the prefusion conformation are provided herein, and include (but are not limited to) negative stain electron microscopy and antibody binding assays using a prefusion conformation specific antibody. The term “pre-F” with reference to a MeV F protein describes a molecule that is a trimeric class I fusion protein stabilized in the prefusion conformation by one or more amino acid substitutions.

MeV F prefusion specific antibody: An antibody that specifically binds to the MeV F protein in a prefusion conformation, but does not specifically bind to the MeV F protein in a postfusion conformation.

MeV hemagglutinin (H) protein: An MeV envelope glycoprotein that is a type II membrane protein and facilitates attachment of MeV to host cell membranes. The full-length H protein has an N-terminal cytoplasmic tail and transmembrane domain (CT and TM, approximately amino acids 1-58), and an ectodomain (approximately amino acids 59-617) including stalk (approximately amino acids 59-179) and head (approximately amino acids 180-617) regions. An exemplary MeV H protein sequence is provided herein as SEQ ID NO: 49 (NCBI Reference Sequence AAA56644.1, which is incorporated by reference herein):

Mspqrdrinafykdnphpkgsrivinrehlmidrpyvllavlfvmflsl
igllaiagirihraaiytaeihkslstnldvtnsiehqvkdvltplfki
igdevglrtpqrftdivkfisdkikfinpdreydfrdltwcinpperik
ldydqycadvaaeelmnalvnstlletrttnqFLAVSKGNCSGPTTIRG
QFSNMSLSLLDLYLGRGYNVSSIVTMTSQGMYGGTYLVEKPNLSSKRSE
LSQLSMYRVFEVGVIRNPGLGAPVFHMTNYLEQPVSNDLSNCMVALGEL
KLAALCHGEDSITIPYQGSGKGVSFQLVKLGVWKSPTDMQSWVPLSTDD
PVIDRLYLSSHRGVIADNQAKWAVPTTRTDDKLRMETCFQQACKGKIQA
LCENPEWAPLKDNRIPSYGVLSVDLSLTVELKIKIASGFGPLITHGSGM
DLYKSNHNNVYWLTIPPMKNLALGVINTLEWIPRFKVSPYLFTVPIKEA
GEDCHAPTYLPAEVDGDVKLSSNLVILPGQDLQYVLATYDTSRVEHAVV
YYVYSPSRSFSYFYPFRLPIKGVPIELQVECFTWDQKLWCRHFCVLADS
ESGGHITHSGMVGMGVSCTVTREDGTNRR

As used herein, MeV H residue positioning is made with reference to the sequence of the set forth as SEQ ID NO: 49.

Mumps: An infectious disease caused by mumps virus. Mumps is characterized by inflammation of the salivary glands, typically the parotid glands. Severe complications of mumps virus infection can occur, such as meningitis, encephalitis, pancreatitis, oophoritis (in females), orchitis (in males) and hearing loss.

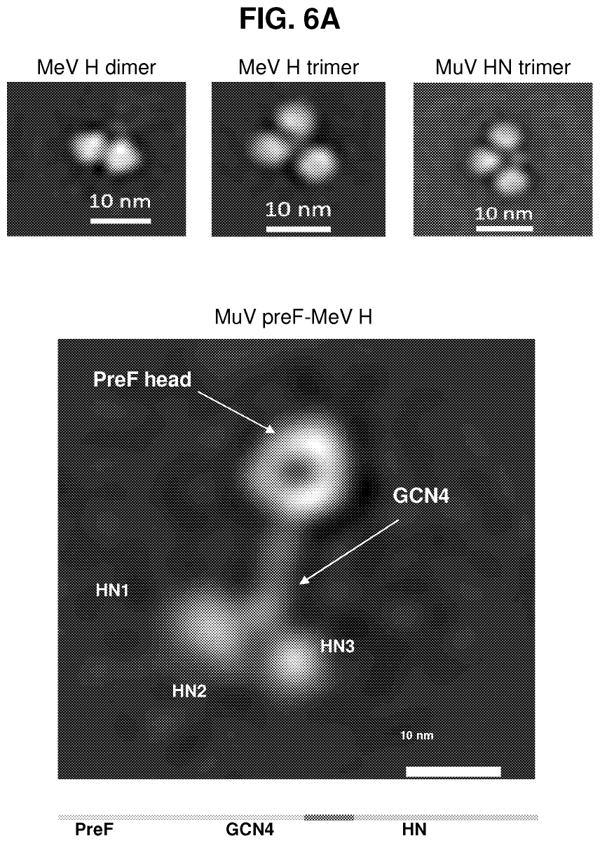

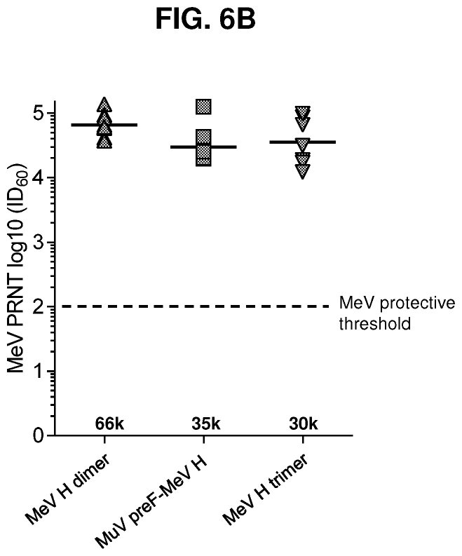

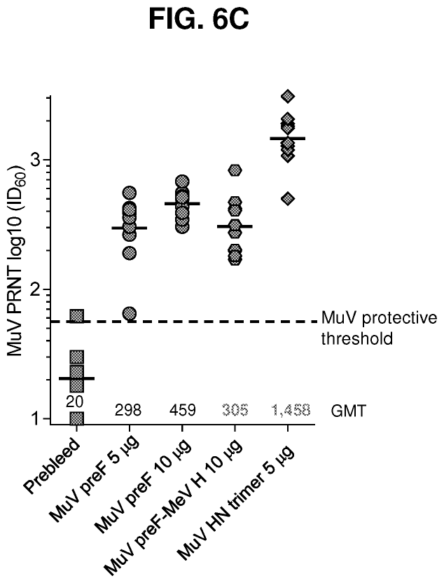

Mumps virus (MuV): A non-segmented, negative-stranded RNA virus of the family Paramyxoviridae, subfamily Paramyxovirinae, genus Rubulavirus that causes mumps disease. Mumps virus genomic RNA contains 7 tandemly linked transcription units that encode open reading frames for the nucleoprotein (N), phosphoprotein (P), V protein, I protein, matrix (M) protein, fusion (F) protein, small hydrophobic (SH) protein, hemagglutinin-neuraminidase (HN) protein, and the large (L) protein. A schematic of the mumps virus genome is shown in FIG. 6. Due to RNA editing by insertion of guanine nucleotides, the P gene (also referred to as the “V/P/I gene”) results in three mRNA transcripts corresponding to the V, P and I proteins. Specifically, faithful transcription of the P gene produces the V protein, insertion of two guanine nucleotides produces an mRNA encoding the P protein, and insertion of four guanine residues results in an mRNA encoding the I protein. The SH gene is the most variable gene amongst different genotypes of MuV and is therefore generally used as the basis for genotyping. There are 12 known genotypes of MuV, designated as genotypes A, B, C, D, F, G, H, I, J, K, L and N, that are currently circulating globally. Most current MuV vaccines are based on genotype A (Jeryl Lynn), genotype B (Urabe-AM9) or undetermined genotype (Leningrad-Zagreb) viruses.

MuV fusion (F) protein: An envelope glycoprotein of MuV that facilitates fusion of viral and cellular membranes. In nature, the F protein from MuV is initially synthesized as a single polypeptide precursor approximately 538 amino acids in length, designated F₀. F₀ includes an N-terminal signal peptide that directs localization to the endoplasmic reticulum, where the signal peptide is proteolytically cleaved. The remaining F₀ residues oligomerize to form a trimer and may be proteolytically processed by a cellular protease to generate two disulfide-linked fragments, F₁ and F₂. In MuV F the cleavage site is located approximately between residues 103/104. The smaller of these fragments, F₂, originates from the N-terminal portion of the F₀ precursor (approximately residues 20-103). The larger of these fragments, F₁, includes the C-terminal portion of the F₀ precursor (approximately residues 104-538) including an extracellular/luminal region (approximately residues 110-483), and a transmembrane and cytosolic regions (approximately residues 484-538). The extracellular portion of the MuV F protein is the MuV F ectodomain, which includes the F₂ protein and the F₁ ectodomain.

The MuV F protein exhibits remarkable sequence conservation within MuV strains. In view of this conservation, the person of ordinary skill in the art can easily compare amino acid positions of different MuV F proteins. Unless context indicates otherwise, the numbering of MuV F amino acids is made with reference to SEQ ID NO: 1 (NCBI Reference Sequence P09458.1, which is incorporated by reference herein):

mkafsvtclgfavfsssicVNINILQQIGYIKQQVRQLSYYSQSSSSYIV
VKLLPNIQPTDNSCEFKSVTQYNKTLSNLLLPIAENINNIASPSPGSRRH
KRFAGIAIGIAALGVATAAQVTAAVSLVQAQTNARAIAAMKNSIQATNRA
IFEVKEGTQQLAIAVQAIQDHINTIMNTQLNNMSCQILDNQLATYLGLYL
TELTTVFQPQLINPALSPISIQALRSLLGSMTPAVVQATLSTSISAAEIL
SAGLMEGQIVSVLLDEMQMIVKINIPTIVTQSNALVIDFYSISSFINNQE
SIIQLPDRILEIGNEQWSYPAKNCKLTRHHIFCQYNEAERLSLESKLCLA
GNISACVFSPIAGSYMRRFVALDGTIVANCRSLTCLCKSPSYPIYQPDHH
AVTTIDLTTCQTLSLDGLDFSIVSLSNITYAENLTISLSQTINTQPIDIS
TELSKVNASLQNAVKYIKESNHQLQSVSVNSKIGAIIVAALVLSILSIII
SLLFCCWAYIATKEIRRINFKTNHINTISSSVDDLIRY

Three MuV F protomers oligomerize in the mature F protein, which adopts a metastable prefusion conformation that is triggered to undergo a conformational change to a postfusion conformation upon contact with a target cell membrane. This conformational change exposes a hydrophobic sequence, known as the fusion peptide, which is located at the N-terminus of the F₁ ectodomain, and which associates with the host cell membrane and promotes fusion of the membrane of the virus, or an infected cell, with the target cell membrane.

An MuV F ectodomain trimer “stabilized in a prefusion conformation” comprises one or more amino acid substitutions, deletions, or insertions compared to a corresponding native MuV F sequence that provide for increased retention of the prefusion conformation compared to MuV F ectodomain trimers formed from a corresponding native MuV F sequence. The “stabilization” of the prefusion conformation can be, for example, energetic stabilization (for example, reducing the energy of the prefusion conformation relative to the postfusion open conformation) and/or kinetic stabilization (for example, reducing the rate of transition from the prefusion conformation to the postfusion conformation). Additionally, stabilization of the MuV F ectodomain trimer in the prefusion conformation can include an increase in resistance to denaturation compared to a corresponding native MuV F sequence. Methods of determining if a MuV F ectodomain trimer is in the prefusion conformation are provided herein, and include (but are not limited to) negative stain electron microscopy and antibody binding assays using a prefusion conformation specific antibody. The term “pre-F” with reference to a MuV F protein describes a molecule that is a trimeric class I fusion protein stabilized in the prefusion conformation by one or more amino acid substitutions.

MuV hemagglutinin-neuraminidase (HN) protein: An MuV envelope glycoprotein that is a type II membrane protein and facilitates attachment of MuV to host cell membranes. The full-length MuV HN protein has an N-terminal cytoplasmic tail and transmembrane domain (CT and TM, approximately amino acids 1-53), and an ectodomain (approximately amino acids 54-582) including stalk (approximately amino acids 54-130) and head regions (approximately amino acids 131-582). An exemplary MuV HN protein sequence is provided herein as SEQ ID NO: 50 (NCBI Reference Sequence AQT03695.1, which is incorporated by reference herein):

mepskfftisdsatfapgpvsnaadkktfrtcfrilvlsvqavtlilviv
tlgelvrmindqglsnqlssitdkiresatmiasavgvmnqvihgvtvsl
plqiegnqnqllatlaticasqkqvsncstNIPLVNDLRFINGINKFIIE
DYATHDFSIGHPLNMPSFIPTATSPNGCTRIPSFSLGKTHWCYTHNVINA
NCKDHTSSNQYVSMGILVQTASGYPMFKTLKIQYLSDGLNRKSCSIATVP
DGCAMYCYVSTQLETDDYAGSSPPTQKLTLLFYNDTVTERTISPSGLEGN
WATLVPGVGSGIYFENKLIEPAYGGVLPNSTLGVKSAREFFRPVNPYNPC
SGPQQDLDQRALRSYFPSYFSNRRIQSAFLVCAWNQILVTNCELVVPSSN
QTMMGAEGRVLLINNRLLYYQRSTSWWPYELLYEISFTFTNSGPSSVNMS
WIPIYSFTRPGSGNCSGENVCPTACVSGVYLDPWPLTPYSHQSGINRNFY
FTGALLNSSTTRVNPTLYVSALNNLKVLAPYGTQGLFASYTTTTCFQDTG
DASVYCVYIMELASNIVGEFQILPVLTRLTIT

As used herein, MuV HN residue positioning is made with reference to the sequence of the set forth as SEQ ID NO: 50.

MuV F prefusion specific antibody: An antibody that specifically binds to the MuV F protein in a prefusion conformation, but does not specifically bind to the MuV F protein in a postfusion conformation.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence.

For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Prime-boost vaccination: An immunotherapy including administration of a first immunogenic composition (the primer vaccine) followed by administration of a second immunogenic composition (the booster vaccine) to a subject to induce an immune response. The primer vaccine and/or the booster vaccine include a vector (such as a viral vector, RNA, or DNA vector) expressing the antigen to which the immune response is directed. The booster vaccine is administered to the subject after the primer vaccine; a suitable time interval between administration of the primer vaccine and the booster vaccine, and examples of such timeframes are disclosed herein. In some embodiments, the primer vaccine, the booster vaccine, or both primer vaccine and the booster vaccine additionally include an adjuvant. In one non-limiting example, the primer vaccine is a DNA-based vaccine (or other vaccine based on gene delivery), and the booster vaccine is a protein subunit or protein nanoparticle based vaccine.

Protein nanoparticle: A self-assembling, multi-subunit, protein-based polyhedron shaped structure. The subunits are each composed of proteins or polypeptides (for example a glycosylated polypeptide), and, optionally of single or multiple features of the following: nucleic acids, prosthetic groups, organic and inorganic compounds. Non-limiting examples of protein nanoparticles include ferritin nanoparticles (see, e.g., Zhang, Y. Int. J. Mol. Sci., 12:5406-5421, 2011, incorporated by reference herein), encapsulin nanoparticles (see, e.g., Sutter et al., Nature Struct. and Mol. Biol., 15:939-947, 2008, incorporated by reference herein), Sulfur Oxygenase Reductase (SOR) nanoparticles (see, e.g., Urich et al., Science, 311:996-1000, 2006, incorporated by reference herein), lumazine synthase nanoparticles (see, e.g., Zhang et al., J. Mol. Biol., 306: 1099-1114, 2001) or pyruvate dehydrogenase nanoparticles (see, e.g., Izard et al., PNAS 96: 1240-1245, 1999, incorporated by reference herein). Ferritin, encapsulin, SOR, lumazine synthase, and pyruvate dehydrogenase are monomeric proteins that self-assemble into a globular protein complexes that in some cases consists of 24, 60, 24, 60, and 60 protein subunits, respectively. In some examples, ferritin, encapsulin, SOR, lumazine synthase, or pyruvate dehydrogenase monomers are linked to a recombinant MuV or MeV F ectodomain and self-assemble into a protein nanoparticle presenting the recombinant MuV or MeV F ectodomain trimer on its surface, which can be administered to a subject to stimulate an immune response to the antigen.

Recombinant: A recombinant nucleic acid molecule is one that has a sequence that is not naturally occurring, for example, includes one or more nucleic acid substitutions, deletions or insertions, and/or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques.

A recombinant virus is one that includes a genome that includes a recombinant nucleic acid molecule.

A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell, or into the genome of a recombinant virus.

Sequence identity: The similarity between amino acid sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide will possess a relatively high degree of sequence identity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid sequence of interest. Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Signal Peptide: A short amino acid sequence (e.g., approximately 18-25 amino acids in length) that directs newly synthesized secretory or membrane proteins to and through membranes (for example, the endoplasmic reticulum membrane). Signal peptides are typically located at the N-terminus of a polypeptide and are removed by signal peptidases after the polypeptide has crossed the membrane. Signal peptide sequences typically contain three common structural features: an N-terminal polar basic region (n-region), a hydrophobic core, and a hydrophilic c-region). An exemplary signal peptide sequence is set forth as MKAFSVTCLSFAVFSSSIC (residues 1-19 of SEQ ID NO: 2).

Specifically bind: When referring to the formation of an antibody:antigen protein complex, or a protein:protein complex, refers to a binding reaction which determines the presence of a target protein, peptide, or polysaccharide (for example, a glycoprotein), in the presence of a heterogeneous population of proteins and other biologics. Thus, under designated conditions, a particular antibody or protein binds preferentially to a particular target protein, peptide or polysaccharide (such as an antigen present on the surface of a pathogen, for example, an antigenic site at the membrane distal apex of the prefusion conformation of the MuV F or MeV F ectodomain timer) and does not bind in a significant amount to other proteins or polysaccharides present in the sample or subject or to alternative conformations of the same protein (for example, the postfusion conformation of the MuV or MeV F proteins). Specific binding can be determined by methods known in the art. A first protein or antibody specifically binds to a target protein when the interaction has a K_{D} of less than 10⁻⁶ Molar, such as less than 10⁻⁷ Molar, less than 10⁻⁸ Molar, less than 10⁻⁹, or even less than 10⁻¹⁰ Molar.

Soluble protein: A protein capable of dissolving in aqueous liquid at room temperature and remaining dissolved. The solubility of a protein may change depending on the concentration of the protein in the water-based liquid, the buffering condition of the liquid, the concentration of other solutes in the liquid, for example salt and protein concentrations, and the heat of the liquid. In several embodiments, a soluble protein is one that dissolves to a concentration of at least 0.5 mg/ml in phosphate buffered saline (pH 7.4) at room temperature and remains dissolved for at least 48 hours.

Subject: Living multi-cellular vertebrate organisms, a category that includes human and non-human mammals. In an example, a subject is a human. In a particular example, the subject is a newborn infant. In an additional example, a subject is selected that is in need of inhibiting of a MuV or MeV infection. For example, the subject is either uninfected and at risk of MuV or MeV infection or is infected in need of treatment.

T4 fibritin trimerization domain: Also referred to as a “foldon” domain, the T4 fibritin trimerization domain comprises an amino acid sequence that naturally forms a trimeric structure. In some examples, a T4 fibritin trimerization domain can be included in the amino acid sequence of a disclosed recombinant protein so that the antigen will form a trimer. In one example, a T4 fibritin trimerization domain comprises the amino acid sequence set forth as GYIPEAPRDGQAYVRKDGEWVLLSTFL (SEQ ID NO: 34). Several embodiments include a T4 fibritin trimerization domain that can be cleaved from a purified protein, for example by incorporation of a thrombin cleave site adjacent to the T4 fibritin trimerization domain that can be used for cleavage purposes.

Transmembrane domain: An amino acid sequence that inserts into a lipid bilayer, such as the lipid bilayer of a cell or virus or virus-like particle. A transmembrane domain can be used to anchor an antigen to a membrane. In some examples a transmembrane domain is a MuV F transmembrane domain. In other examples a transmembrane domain is a MeV F transmembrane domain.

Under conditions sufficient for: A phrase that is used to describe any environment that permits a desired activity.

Vaccine: A preparation of immunogenic material capable of stimulating an immune response, administered for the prevention, amelioration, or treatment of infectious or other types of disease. The immunogenic material may include attenuated or killed microorganisms (such as bacteria or viruses), or antigenic proteins, peptides, or DNA derived from them. A vaccine may include a disclosed immunogen (such as a recombinant MuV or MeV F ectodomain trimer or nucleic acid molecule encoding same), a virus, a cell or one or more cellular constituents. Vaccines may elicit both prophylactic (preventative or protective) and therapeutic responses. Methods of administration vary according to the vaccine, but may include inoculation, ingestion, inhalation or other forms of administration. Vaccines may be administered with an adjuvant to boost the immune response. In one specific, non-limiting example, a vaccine prevents and/or reduces the severity of the symptoms associated with MuV infection and/or decreases the viral load compared to a control. In another specific, non-limiting example, a vaccine prevents and/or reduces the severity of the symptoms associated with MeV infection and/or decreases the viral load compared to a control.

Vector: An entity containing a DNA or RNA molecule bearing a promoter(s) that is operationally linked to the coding sequence of an antigen(s) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant nucleic acid vectors having at least some nucleic acid sequences derived from one or more viruses.

Virus-like particle (VLP): A non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Virol. 68:4503-4505; Vincente, J. Invertebr Pathol., 2011; Schneider-Ohrum and Ross, Curr. Top. Microbiol. Immunol., 354: 53073, 2012).

### II. Immunogens

### A. Recombinant MuV F Ectodomain Trimers

Recombinant MuV F ectodomain trimers are disclosed herein that are modified from a native form (e.g., by introduction of one or more amino acid substitutions) to be stabilized in a prefusion conformation. As described in the Examples, embodiments of the disclosed MuV F ectodomain trimers have been selected through multiple rounds of structure based design for optimized solubility, stability, expression, and immunogenicity. The recombinant MuV F ectodomain trimers are useful to induce an immune response in a vertebrate animal (such humans) to MuV. Exemplary embodiments are shown to produce a superior immune response in an animal model compared to corresponding MuV F ectodomain trimers that are not stabilized in the prefusion conformation.

In some embodiments, the immunogen comprises a recombinant MuV F ectodomain trimer comprising protomers comprising one or more amino acid substitutions or deletions that stabilize the MuV F ectodomain trimer in the prefusion conformation.

In some embodiments, the immunogen comprises a recombinant MuV F ectodomain trimer stabilized in a prefusion conformation by one or more amino acid substitutions in protomers of the trimer, the amino acid substitutions comprising cysteine substitutions that form a non-natural disulfide bond to stabilize the MuV F ectodomain trimer in the prefusion conformation. A non-natural disulfide bond is one that does not occur in a native MuV F protein, and is introduced by protein engineering (e.g., by including one or more substituted cysteine residues that form the non-natural disulfide bond). For example, in some embodiments, any of the disclosed recombinant MuV F proteins can be stabilized in a prefusion conformation by any one of 1, 2, 3, 4, 5, 6, 7, 8, 9, or 10 non-natural disulfide bonds.

The cysteine residues that form the disulfide bond can be introduced into a native MuV F sequence by one or more amino acid substitutions. For example, in some embodiments, a single amino acid substitution introduces a cysteine that forms a disulfide bond with a cysteine residue present in the native MuV F sequence. Alternately, two cysteine residues can be introduced into a native MuV F sequence to form the disulfide bond. The location of the cysteine (or cysteines) of the non-natural disulfide bond can be determined by the person of ordinary skill in the art using the disclosed structure of the MuV F ectodomain trimer in a prefusion conformation.

The amino acid positions of the cysteines are typically within a sufficiently close distance for formation of a disulfide bond in the prefusion conformation of the MuV F protein trimer. Methods of using three-dimensional structure data (for example, as provided in Table 1) to determine if two residues are within a sufficiently close distance to one another for disulfide bond formation are known (see, e.g., Peterson et al., Protein engineering, 12:535-548, 1999 and Dombkowski, Bioinformatics, 19:1852-1853, 3002 (disclosing DISULFIDE BY DESIGN™), each of which is incorporated by reference herein). Residues can be selected manually, based on the three dimensional structure of the MuV F trimer in a prefusion conformation provided herein, or a software, such as DISULFIDEBYDESIGN™, can be used. Without being bound by theory, ideal distances for formation of a disulfide bond are generally considered to be about −5.6 Å for Cα-Cα distance, ~2.02 Å for Sγ-Sγ distance, and 3.5-4.25 Å for Cβ-Cβ distance (using the optimal rotomer). The person of ordinary skill in the art will appreciate that variations from these distances are included when selecting residues in a three dimensional structure that can be substituted for cysteines for introduction of a disulfide bond. For example, in some embodiments the selected residues have a Cα-Cα distance of less than 7.0 Å and/or a Cβ-Cβ distance of less than 4.7 Å. In some embodiments the selected residues have a Cα-Cα distance of from 2.0-8.0 Å and/or a Cβ-Cβ distance of from 2.0-6.0 Å.

In some embodiments, the protomers of the recombinant MuV F ectodomain trimer comprise cysteine substitutions at MuV F positions 86 and 215 (such as N86C and A215C substitutions) that form a non-natural intra-protomer disulfide bond for stabilization in the prefusion conformation.

In some embodiments, the protomers of the recombinant MuV F ectodomain trimer comprise cysteine substitutions at MuV F positions 155 and 161 (such as K155C and L161C substitutions) that form a non-natural intra-protomer disulfide bond for stabilization in the prefusion conformation.

In some embodiments, the protomers of the recombinant MuV F ectodomain trimer comprise cysteine substitutions at MuV F positions 165 and 231 (such as V165C and M231C substitutions) that form a non-natural intra-protomer disulfide bond for stabilization in the prefusion conformation.

In some embodiments, the protomers of the recombinant MuV F ectodomain trimer comprise cysteine substitutions at MuV F positions 206 and 223 (such as V206C and A223C substitutions) that form a non-natural intra-protomer disulfide bond for stabilization in the prefusion conformation.

In some embodiments, the protomers of the recombinant MuV F ectodomain trimer comprise cysteine substitutions at MuV F positions 209 and 214 (such as P209C and P214C substitutions) that form a non-natural intra-protomer disulfide bond for stabilization in the prefusion conformation.

In some embodiments, the protomers of the recombinant MuV F ectodomain trimer comprise cysteine substitutions at MuV F positions 221 and 255 (such as I221C and M255C substitutions) that form a non-natural intra-protomer disulfide bond for stabilization in the prefusion conformation.

Any of the above recombinant MuV F proteins can further comprise modification to eliminate the protease cleavage site between the F₁ and F₂ polypeptides to generate a “single chain” recombinant F protein. For example, any of the above recombinant MuV proteins can comprise deletion of MuV F positions 101-103 with positions 100 and 104 fused by a peptide linker. This modification removes the F2/F1 furin cleavage site and also removed the first residue of the fusion peptide (which is hydrophobic). Any suitable peptide linker may be used that fuses the F2 and F1 ectodomain and allows folding of the F ectodomain into the prefusion conformation. In some embodiments, the peptide linker is a glycine, serine, or glycine-serine peptide linker. In some embodiments, the peptide linker is a Gly-Gly-Gly linker.

In a non-limiting example, a recombinant MuV F ectodomain trimer is provided that includes protomers with V206C and A223C substitutions to form a non-natural disulfide bond and a deletion of MuV F positions 101-103 with positions 100 and 104 fused by a Gly-Gly-Gly peptide linker.

In several embodiments, the protomers of the recombinant MuV F ectodomain can comprise one or more additional amino acid substitutions, for example, to increase stabilization of the prefusion conformation, or for other purposes, such as to increase solubility or to reduce and unwanted immune response.

The above-listed non-native disulfide bonds stabilize the membrane-distal portion of the MuV F ectodomain in its prefusion conformation. Any of these mutations can be combined with modifications to the membrane proximal portion (such as the stem) of the MuV F ectodomain, for example, to increase trimerization of the ectodomain.

In several embodiments, the N-terminal position of the recombinant F2 polypeptide in the protomer can be one of MuV F positions 20-30 (such as position 20), and the C-terminal position of the F1 ectodomain can be from the stem region of the ectodomain, such as one of MuV F positions 469-483 (such as position 476).

In a non-limiting example, a recombinant MuV F ectodomain trimer is provided that includes protomers including MuV positions 20-476 with V206C and A223C substitutions to form a non-natural disulfide bond and a deletion of MuV F positions 101-103 with positions 100 and 104 fused by a Gly-Gly-Gly peptide linker.

Non-limiting examples of protomers of a MuV F ectodomain trimer including amino acid substitutions for stabilization in the prefusion conformation are provided herein. In some embodiments, the protomers of the MuV F ectodomain trimer comprise an amino acid sequence at least 90% identical to residues 20-483 of any one of SEQ ID NOs: 3-8, residues 20-476 of any one of SEQ ID NOs: 11-16, 26, or 51, or residues 20-469 of any one of SEQ ID NOs: 19-24; wherein the protomers comprise the one or more amino acid substitutions that stabilize the MuV F ectodomain trimer in the prefusion conformation. In some embodiments, the protomers of the MuV F ectodomain trimer comprise residues 20-483 of any one of SEQ ID NOs: 3-8, residues 20-476 of any one of SEQ ID NOs: 11-16, 26, or 51, or residues 20-469 of any one of SEQ ID NOs: 19-4.

In several embodiments, the recombinant MuV F ectodomain trimer is a soluble protein complex, for example, for use as a recombinant subunit vaccine. In several such embodiments, the protomers of the recombinant MuV F ectodomain trimer can each comprise a C-terminal linkage to a trimerization domain, such as a GCN4 trimerization domain or a T4 fibritin trimerization domain, or both. The trimerization domain promotes trimerization and stabilization of the membrane proximal aspect of the recombinant MuV F ectodomain trimer. For example, a C-terminal residue of the protomers of the recombinant MuV F ectodomain trimer (such as a residue of the stem region of the trimer) can be directly linked to the trimerization domain, or indirectly linked to the trimerization domain via a peptide linker. Exemplary linkers include glycine and glycine-serine linkers. Non-limiting examples of exogenous multimerization domains that promote stable trimers of soluble recombinant proteins include: the GCN4 leucine zipper, a T4 fibritin trimerization domain, the trimerization motif from the lung surfactant protein (Hoppe et al. 1994 FEBS Lett 344:191-195) or collagen (McAlinden et al. 2003 J Biol Chem 278:42200-42207), any of which can be linked to the C-terminus of the protomers of a recombinant MuV F ectodomain to promote trimerization, as long as the recombinant MuV F ectodomain trimer retains the prefusion conformation. In some examples, the protomers of the recombinant MuV F ectodomain trimer can be linked to a MuV trimerization domain, for example, each protomer in the trimer can include a C-terminal linkage to the GCN4 trimerization domain, such as a linkage to any one of MuV F positions 469-483, such as MuV F position 469, MuV F position 476, or MuV F position 483. In specific examples, the GCN4 trimerization domain comprises or consists of the amino acid sequence IEDKIEEILSKIYHIENEIARIKKLIGEAP (SEQ ID NO: 33). In specific examples, the T4 fibritin trimerization domain comprises or consists of the amino acid sequence GYIPEAPRDGQAYVRKDGEWVLLSTFL (SEQ ID NO: 34). In specific examples, the GCN4 trimerization domain fused to the fibritin trimerization domain comprises or consists of the amino acid sequence

(SEQ ID NO: 35)
IEDKIEEILSKIYHIENEIARIKKLIGEAPGSGYIPEAPRDGQAYVRKDG
EWVLLSTFL.

In a non-limiting example, a recombinant MuV F ectodomain trimer is provided that includes protomers including MuV positions 20-476 with V206C and A223C substitutions to form a non-natural disulfide bond, a deletion of MuV F positions 101-103 with positions 100 and 104 fused by a Gly-Gly-Gly peptide linker, and a GCN4 trimerization domain linked to the C-terminus of the protomers in the ectodomain.

Non-limiting examples of protomers of a MuV F ectodomain trimer including amino acid substitutions for stabilization in the prefusion conformation as well as a C-terminal linkage to a trimerization domain are provided herein. In some embodiments, the protomers of the MuV F ectodomain trimer comprise an amino acid sequence at least 90% identical to residues 20-513 of any one of SEQ ID NOs: 3-8, residues 20-506 of any one of SEQ ID NOs: 11-16, 26, or 51, or residues 20-499 of any one of SEQ ID NOs: 19-24; and wherein the protomers comprise the one or more amino acid substitutions that stabilize the MuV F ectodomain trimer in the prefusion conformation. In some embodiments, the protomers of the MuV F ectodomain trimer comprise residues 20-513 of any one of SEQ ID NOs: 3-8, residues 20-506 of any one of SEQ ID NOs: 11-16, 26, or 51, or residues 20-499 of any one of SEQ ID NOs: 19-24.

In some embodiments, the recombinant MuV F ectodomain trimer can be a membrane anchored protein complex, for example, for use in an attenuated virus or virus like particle vaccine. Membrane anchoring can be accomplished, for example, by C-terminal linkage of the protomers of the recombinant MuV F ectodomain trimer to a transmembrane domain and optionally a cytoplasmic tail, such as a MuV F transmembrane domain and cytoplasmic tail. In some embodiments, one or more peptide linkers (such as a gly-ser linker, for example, a 10 amino acid glycine-serine peptide linker can be used to link the protomers of the recombinant MuV F ectodomain trimer to the transmembrane domain. A non-limiting example of a transmembrane domain for use with the disclosed embodiments includes a MuV F transmembrane domain, such as GAI IVAALVLSILSIIISLLFCCW (SEQ ID NO: 44). A non-limiting example of a transmembrane domain and cytoplasmic tail for use with the disclosed embodiments includes a MuV F transmembrane domain and cytoplasmic tail, such as GAIIVAALVLSILSIIISLLFCCWAYIATKEIRRINFKTNHINTISSSVDDLIRY (SEQ ID NO: 99).

Native MuV F proteins from different MuV strains, as well as nucleic acid sequences encoding such proteins and methods, are known and can be altered using the description provided herein to generate a recombinant MuV F ectodomain trimer.

The recombinant MuV F ectodomain trimer can be derivatized or linked to another molecule (such as another peptide or protein). In general, the recombinant MuV F ectodomain is derivatized such that the binding to neutralizing antibodies to a trimer of the recombinant MuV F protein is not affected adversely by the derivatization or labeling. For example, the recombinant MuV F ectodomain can be functionally linked (by chemical coupling, genetic fusion, non-covalent association or otherwise) to one or more other molecular entities, such as a cater protein, an antibody, a heterologous protein, or a detection tag.

In some embodiments, the recombinant MuV F ectodomain trimers are fused to one or more MuV HN ectodomains, such as the ectodomain head of the MuV HN sequence set forth as:

MuV HN ectodomain head (genotype G,
Arkansas16, GenBank ARM65482.1
(SEQ ID NO: 30)
NIPLVNDLRFINGINKFIIEDYATHDFSIGHPLNMPSFIPTATSPNGCTR
IPSFSLGKTHWCYTHNVINANCKDHTSSNQYVSMGILVQTASGYPMFKTL
KIQYLSDGLNRKSCSIATVPDGCAMYCYVSTQLETDDYAGSSPPTQKLTL
LFYNDTVTERTISPSGLEGNWATLVPGVGSGIYFENKLIFPAYGGVLPNS
TLGVKSAREFFRPVNPYNPCSGPQQDLDQRALRSYFPSYFSNRRIQSAFL
VCAWNQILVTNCELVVPSSNQTMMGAEGRVLLINNRLLYYQRSTSWWPYE
LLYEISFTFTNSGPSSVNMSWIPIYSFTRPGSGNCSGENVCPTACVSGVY
LDPWPLTPYSHQSGINRNFYFTGALLNSSTTRVNPTLYVSALNNLKVLAP
YGTQGLFASYTTTTCFQDTGDASVYCVYIMELASNIVGEFQILPVLTRLT
IT

In some embodiments, the recombinant MuV F ectodomain trimers are fused to one or more MuV HN ectodomains, such as the ectodomain stalk and head of any one of MuV HN positions 54-130 to MuV HN position 582 (such as MuV HN positions 54-63 to MuV HN position 582, for example, positions 54-582, 61-582, 63-582, or 55-582). In some embodiments, the recombinant MuV F ectodomain trimers are fused to one or more MuV HN ectodomains, such as the ectodomain stalk and head of the sequence set forth as residues 22-550 of SEQ ID NO: 90, residues 22-543 of SEQ ID NO: 91, residues 22-541 of SEQ ID NO: 92, or residues 22-549 of SEQ ID NO: 93.

For example, the protomers of the recombinant MuV F ectodomain trimer are each fused to a MuV HN ectodomain. The fusion can be direct or via a peptide linker. In some embodiments, the MuV HN ectodomain can be fused, directly or indirectly via a peptide linker, to the C-terminus of the protomers of the MuV F ectodomain trimer. In some such embodiments, the MuV HN ectodomain can be fused, directly or indirectly via a peptide linker, to the C-terminus of a trimerization domain (such as a GCN4 or T4 fibritin trimerization domain) fused to the C-terminus of the protomers of the MuV F ectodomain trimer. In some such embodiments, the protomers of the MuV F ectodomain trimer linked to the trimerization domain and the MuV HN ectodomain comprise an amino acid sequence set forth as residues 20-966 of SEQ ID NO: 27, or an amino acid sequence at least 90% identical to residues 20-966 of SEQ ID NO: 27.

In some embodiments, the recombinant MuV F ectodomain trimers are fused to one or more MeV H ectodomains, such as the ectodomain head of the H sequence set forth as:

MeV H ectodomain head
(SEQ ID NO: 31)
FLAVSKGNCSGPTTIRGQFSNMSLSLLDLYLGRGYNVSSIVTMTSQGMYG
GTYLVEKPNLSSKRSELSQLSMYRVFEVGVIRNPGLGAPVFHMTNYLEQP
VSNDLSNCMVALGELKLAALCHGEDSITIPYQGSGKGVSFQLVKLGVWKS
PADMQSWVPLSTDDPVIDRLYLSSHRGVIADNQAKWAVPTTRTDDKLRME
TCFQQACKGKIQTLCENPEWAPLKDNRIPSYGVLSVDLSLTVELKIKIAS
GFGPLITHGSGMDLYKSNHNNVYWLTIPPMKNLALGVINTLEWIPRFKVS
PYLFTVPIKEAGEDCHAPTYLPAEVDGDVKLSSNLVILPGQDLQYVLATY
DTSRVEHAVVYYVYSPGRSFSYFYPFRLPIKGVPIELQVECFTWDQKLWC
RHFCVLADSESGGHITHSGMVGMGVSCTVTREDGTNRR
MeV H ectodomain head
(SEQ ID NO: 32)
ADVAAEELMNALVNSTLLEARATNQFLAVSKGNCSGPTTIRGQFSNMSLS
LLDLYLSRGYNVSSIVTMTSQGMYGGTYLVGKPNLSSKGSELSQLSMHRV
FEVGVIRNPGLGAPVFHMTNYFEQPVSNDFSNCMVALGELKFAALCHRED
SITIPYQGSGKGVSFQLVKLGVWKSPTDMRSWVPLSTDDPVIDRLYLSSH
RGVIADNQAKWAVPTTRTDDKLRMETCFQQACKGKNQALCENPEWAPLKD
NRIPSYGVLSVNLSLTVELKIKIASGFGPLITHGSGMDLYKTNHNNVYWL
TIPPMKNLALGVINTLEWIPRFKVSPNLFTVPIKEAGEDCHAPTYLPAEV
DGDVKLSSNLVILPGQDLQYVLATYDTSRVEHAVVYYVYSPSRSFSYFYP
FRLPIKGVPIELQVECFTWDKKLWCRHFCVLADSESGGHITHSGMVGMGV
SCTVTREDGTNRR

In some embodiments, the recombinant MuV F ectodomain trimers are fused to one or more MeV H ectodomains, such as the ectodomain stalk and head of any one of MeV H positions 59-179 to MeV H position 617 (such as any one of MeV H positions 59-67 to MeV H position 617, for example positions 59-617, 62-617, 60-617, or 67-617). In some embodiments, the recombinant MuV F ectodomain trimers are fused to one or more MeV H ectodomains, such as the ectodomain stalk and head of the sequence set forth as residues 22-580 of SEQ ID NO: 86, residues 22-577 of SEQ ID NO: 87, residues 22-579 of SEQ ID NO: 88, or residues 22-572 of SEQ ID NO: 89.

For example, the protomers of the recombinant MuV F ectodomain trimer stabilized in the prefusion conformation are each fused to a MeV H ectodomain. The fusion can be direct or via a peptide linker. In some embodiments, the MeV H ectodomain can be fused, directly or indirectly via a peptide linker, to the C-terminus of the protomers of the prefusion MuV F ectodomain trimer. In some such embodiments, the MeV H ectodomain can be fused, directly or indirectly via a peptide linker, to the C-terminus of a trimerization domain (such as a GCN4 or T4 fibritin trimerization domain) fused to the C-terminus of the protomers of the MuV F ectodomain trimer. In some such embodiments, the protomers of the MuV F ectodomain trimer linked to the trimerization domain and the MeV H ectodomain comprise an amino acid sequence set forth as residues 21-981 of SEQ ID NO: 28 or residues 20-1006 of SEQ ID NO: 29, or an amino acid sequence at least 90% identical to residues 21-981 of SEQ ID NO: 28 or residues 20-1006 of SEQ ID NO: 29.

Non-limiting examples of sequences containing a MuV F ectodomain with amino acid substitutions for stabilization in a refusion conformation are provided as follows:

MuV F V206C/A223C-GGG-483-GCN4
(SEQ ID NO: 3)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttCfqpqltnpalspisiqClrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyikesnhqlqsvsvn
skiIEDKIEEILSKIYHIENEIARIKKLIGEAP
MuV F N86C/A215C-GGG-483-GCN4
(SEQ ID NO: 4)
mkafsvtclsfavfsssicvninilqqigyikqqvrqlsyysqssssyivvkllpniqptddscefksvtqynktlsnll
lpiaeCinniaspspgsrrhGGGagiaigiaalgvataaqvtaavslvqaqtnaraiaamknsiqatnravfevkegtqq
laiavqaiqdhintimntqlnnmscqildnqlatslglyltelttvfqpqltnpClspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyikesnhqlqsvsvn
skiIEDKIEEILSKIYHIENEIARIKKLIGEAP
MuV F P209C/P214C-GGG-483-GCN4
(SEQ ID NO: 5)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttvfqCqltnCalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyikesnhqlqsvsvn
skiIEDKIEEILSKIYHIENEIARIKKLIGEAP
MuV F K155C/L161C-GGG-483-GCN4
(SEQ ID NO: 6)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevCegtqq
Caiavqaiqdhintimntqlnnmscqildnqlatslglyltelttvfqpqltnpalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyikesnhqlqsvsvn
skiIEDKIEEILSKIYHIENEIARIKKLIGEAP
MuV F V165C/M231C-GGG-483-GCN4
(SEQ ID NO: 7)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiaCqaiqdhintimntqlnnmscqildnqlatslglyltelttvfqpqltnpalspisiqalrsllgsCtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyikesnhqlqsvsvn
skiIEDKIEEILSKIYHIENEIARIKKLIGEAP
MuV F I221C/M255C-GGG-483-GCN4
(SEQ ID NO: 8)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttvfqpqltnpalspisCqalrsllgsmtpavvqatl
stsisaaeilsaglCegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyikesnhqlqsvsvn
skiIEDKIEEILSKIYHIENEIARIKKLIGEAP
MuV F V206C/A223C-GGG-476-GCN4
(SEQ ID NO: 11)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttCfqpqltnpalspisiqClrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyikesnhqlqs
IEDKIEEILSKIYHIENEIARIKKLIGEAP
MuV F N86C/A215C-GGG-476-GCN4
(SEQ ID NO: 12)
mkafsvtclsfavfsssicvninilqqiqyikqqvrqlsyysqssssyivvkllpniqptddscefksvtqynktlsnll
lpiaeCinniaspspgsrrhGGGagiaigiaalgvataaqvtaavslvqaqtnaraiaamknsiqatnravfevkegtqq
laiavqaiqdhintimntqlnnmscqildnqlatslglyltelttvfqpqltnpClspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyikesnhqlqs
IEDKIEEILSKIYHIENEIARIKKLIGEAP
MuV F P209C/P214C-GGG-476-GCN4
(SEQ ID NO: 13)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttvfqCqltnCalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyikesnhqlqs
IEDKIEEILSKIYHIENEIARIKKLIGEAP
MuV F K155C/L161C-GGG-476-GCN4
(SEQ ID NO: 14)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevCegtqq
Caiavqaiqdhintimntqlnnmscqildnqlatslglyltelttvfqpqltnpalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyikesnhqlqs
IEDKIEEILSKIYHIENEIARIKKLIGEAP
MuV F V165C/M231C-GGG-476-GCN4
(SEQ ID NO: 15)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiaCqaiqdhintimntqlnnmscqildnqlatslglyltelttvfqpqltnpalspisiqalrsllgsCtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyikesnhqlqs
IEDKIEEILSKIYHIENEIARIKKLIGEAP
MuV F I221C/M255C-GGG-476-GCN4
(SEQ ID NO: 16)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttvfqpqltnpalspisCqalrsllgsmtpavvqatl
stsisaaeilsaglCegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyikesnhqlqs
IEDKIEEILSKIYHIENEIARIKKLIGEAP
MuV F V206C/A223C-GGG-469-GCN4
(SEQ ID NO: 19)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttCfqpqltnpalspisiqClrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyike
IEDKIEEILSKIYHIENEIARIKKLIGEAP
MuV F N86C/A215C-GGG-469-GCN4
(SEQ ID NO: 20)
mkafsvtclsfavfsssicvninilqqiqyikqqvrqlsyysqssssyivvkllpniqptddscefksvtqynktlsnll
lpiaeCinniaspspgsrrhGGGagiaigiaalgvataaqvtaavslvqaqtnaraiaamknsiqatnravfevkegtqq
laiavqaiqdhintimntqlnnmscqildnqlatslglyltelttvfqpqltnpClspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyike
IEDKIEEILSKIYHIENEIARIKKLIGEAP
MuV F P209C/P214C-GGG-469-GCN4
(SEQ ID NO: 21)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttvfqCqltnCalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyike
IEDKIEEILSKIYHIENEIARIKKLIGEAP
MuV F K155C/L161C-GGG-469-GCN4
(SEQ ID NO: 22)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevCegtqq
CaiavqaiqdhintimntqlnnmscqildnqlatslglyItelttvfqpqltnpalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyike
IEDKIEEILSKIYHIENEIARIKKLIGEAP
MuV F V165C/M231C-GGG-469-GCN4
(SEQ ID NO: 23)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiaCqaiqdhintimntqlnnmscqildnqlatslglyltelttvfqpqltnpalspisiqalrsiIgsCtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyike
IEDKIEEILSKIYHIENEIARIKKLIGEAP
MuV F I221C/M255C-GGG-469-GCN4
(SEQ ID NO: 24)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttvfqpqltnpalspisCqalrsllgsmtpavvqatl
stsisaaeilsaglCegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivsisnityaenltislsqtintqpidistelskvnaslqnavkyike
IEDKIEEILSKIYHIENEIARIKKLIGEAP
MuV-JL F 206C/A223C-GGG-476-GCN4
(SEQ ID NO: 26)
mkafsvtclsfavfsssicvninilqqigyikqqvrqlsyysqssssyVvvkllpniqptdNscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttCfqpqlInpalspisiqClrsllgsmtpavvqatl
stsisaaeilsaglmegqiVsvlldemqmivkinVptivtqsnalvidfysissfinnqesiiqlpdrileigneqwRyp
aknckStrhhifcqyneaerisleTklclagnisacvfspiagsymrrfvaldgtivancrsltclckspsypiyqpdhh
avttidltScqtlsldgldfsivsisnityaenltislsqtintqpidistelskvnaslqnavkyikesnhqlqs
IEDKIEEILSKIYHIENEIARIKKLIGEAP
MuV-IL17 F 206C/A223C-GGG-476-GCN4
(SEQ ID NO: 51)
mkvslvtclgfavfsfsicvninilqqiqyikqqvrqlsyysqssssyivvkllpniqptdnscefksvtqynktlsnll
lpiaeninniaspspgsrrhGGGagiaigiaalgvataaqvtaavslvqaqtnaraiaamknsiqatnravfevkegtqq
laiavqaiqnhintimntqlnnmscqildnqlatslglylteLTTCfqpqlinpalspisiqCLRSllgsmtpavvqatl
stsisaaeilsaglmegqivsvlldemqmivkiniptivtqsnalvidfysissfingqesiiqlpdrileigneqwsyp
aknckltrhnifcqyneaerlslesklclagnisacvfspiagsymrrfvaldgtivancrsltclckspsypiyqpdhh
avttidltacqtlsldgldfsivsisnityaenltisisqtintqpidistelikvnaslqnavkyikesnhqlqs
IEDKIEEILSKIYHIENEIARIKKLIGEAP

The above sequences include an N-terminal signal peptide, a MuV F ectodomain, and a GCN4 trimerization domain. It will be appreciated that an alternative trimerization domain can be used, such as a T4 Fibritin trimerization domain. Additionally, many of the above sequence include a GGG linker to remove the native furin cleavage site separating the F1 and F2 subunits. Alternate glycine linkers can also be used, such as GSG, GGS, or SGG. Additionally, in any of the sequences, the native furin cleavage site can be included instead of the GGG linker. It will be appreciated that the N-terminal signal peptide is removed during cellular processing and is not present in the purified protein. Additionally, the MuV F ectodomain of any of the above sequences can be included in a full-length MuV F protein to provide a membrane anchored version of the prefusion MuV F protein, for example for mRNA immunization.

Non-limiting examples of sequences containing a MuV F ectodomain with amino acid substitutions for stabilization in a prefusion conformation linked to a MuV HN ectodomain or a MeV H ectodomain are provided as follows:

MuV F 206C/223C-GGG-476 + GCN4 + MuV HN_G
(SEQ ID NO: 27)
Mkafsvtclsfavfsssicvninilqqiqyikqqvrqlsyysqssssyiv
vkllpniqptddscefksvtqynktlsnlllpiaeninniaspspgsrrh
GGGagiaigiaalgvataaqvtaavslvqaqtnaraiaamknsiqatnra
vfevkegtqqlaiavqaiqdhintimntqlnnmscqildnqlatslglyl
telttCfqpqltnpalspisiqClrsllgsmtpavvqatlstsisaaeil
saglmegqiisvlldemqmivkiniptivtqsnalvidfysissfinnqe
siiqlpdrileigneqwsypaknckltrhhifcqyneaerlslesklcla
gnisacvfspiagsymrrfvaldgtivancrsltclckspsypiyqpdhh
avttidltacqtlsldgldfsivsisnityaenltislsqtintqpidis
telskvnaslqnavkyikesnhqlqsIEDKIEEILSKIYHIENEIARIKK
LIGEAPGSGGGGGGNiplvndlrfinginkfiiedyathdfsighpinm
psfiptatspngctripsfsigkthwcythnvinanckdhtssnqyvsmg
ilvqtasgypmfktikiqylsdglnrkscsiatvpdgcamycyvstqlet
ddyagsspptqkltllfyndtvtertispsglegnwativpgvgsgiyfe
nklifpayggvlpnstigvksareffrpvnpynpcsgpqqdldqralrsy
fpsyfsnrriqsaflvcawnqilvtncelvvpssnqtmmgaegrvllinn
rllyyqrstswwpyellyeisftftnsgpssvnmswipiysftrpgsgnc
sgenvcptacvsgvyldpwpltpyshqsginrnfyftgallnssttrvnp
tlyvsalnnlkvlapygtqglfasyttttcfqdtgdasvycvyimelasn
ivgefqilpvltrltit
MuV F 206C/223C-GGG-476 + GCN4/Fd + MeV_H
(SEQ ID NO: 28)
Mkafsvtclsfavfsssicvninilqqiqyikqqvrqlsyysqssssyiv
vkllpniqptddscefksvtqynktlsnlllpiaeninniaspspgsrrh
GGGagiaigiaalgvataaqvtaavslvqaqtnaraiaamknsiqatnra
vfevkegtqqlaiavqaiqdhintimntqlnnmscqildnqlatslglyl
telttCfqpqltnpalspisiqClrsllgsmtpavvqatlstsisaaeil
saglmegqiisvlldemqmivkiniptivtqsnalvidfysissfinnqe
siiqlpdrileigneqwsypaknckltrhhifcqyneaerlslesklcla
gnisacvfspiagsymrrfvaldgtivancrsltclckspsypiyqpdhh
avttidltacqtlsldgldfsivsisnityaenltislsqtintqpidis
telskvnaslqnavkyikesnhqlqsIEDKIEEILSKIYHIENEIARIKK
LIGEAPGSGYIPEAPRDGQAYVRKDGEWVLLSTFLGSGGGGGgF
lavskgncsgpttirgqfsnmsislldlylgrgynvssivtmtsqgmygg
tylvekpnlsskrselsqlsmyrvfevgvirnpglgapvfhmtnyleqpv
sndlsncmvalgelklaalchgedsitipyqgsgkgvsfqlvklgvwksp
admqswvplstddpvidrlyisshrgviadnqakwavpttrtddklrmet
cfqqackgkiqticenpewaplkdnripsygvlsvdls1tvelkikiasg
fgplithgsgmdlyksnhnnvywltippmknlalgvintlewiprfkvsp
ylftvpikeagedchaptyipaevdgdvklssnlvilpgqdlqyvlatyd
tsrvehavvyyvyspgrsfsyfypfrlpikgvpielqvecftwdqklwcr
hfcvladsesgghithsgmvgmgvsctvtredgtnrr
MuV F 206C/223C-GGG-476 + GCN4/Fd + MeV_H
(SEQ ID NO: 29)
Mkafsvtclsfavfsssicvninilqqigyikqqvrqlsyysqssssyiv
vkllpniqptddscefksvtqynktlsnlllpiaeninniaspspgsrrh
GGGagiaigiaalgvataaqvtaavslvqaqtnaraiaamknsiqatnra
vfevkegtqqlaiavqaiqdhintimntqlnnmscqildnqlatslglyl
telttCfqpqltnpalspisiqClrsllgsmtpavvqatlstsisaaeil
saglmegqiisvlldemqmivkiniptivtqsnalvidfysissfinnqe
siiqlpdrileigneqwsypaknckltrhhifcqyneaerlslesklcla
gnisacvfspiagsymrrfvaldgtivancrsltclckspsypiyqpdhh
avttidltacqtlsldgldfsivsisnityaenltislsqtintqpidis
telskvnaslqnavkyikesnhqlqsIEDKIEEILSKIYHIENEIARIKK
LIGEAPGSGYIPEAPRDGQAYVRKDGEWVLLSTFLGSGGGGGgA
dvaaeelmnalvnstllearatnqflavskgncsgpttirgqfsnmslsl
ldlylsrgynvssivtmtsqgmyggtylvgkpnlsskgselsqlsmhrvf
evgvirnpglgapvfhmtnyfeqpvsndfsncmvalgelkfaalchreds
itipyqgsgkgvsfqlvklgvwksptdmrswvplstddpvidrlyisshr
gviadnqakwavpttrtddklrmetcfqqackgknqalcenpewaplkdn
ripsygvlsvnlsitvelkikiasgfgplithgsgmdlyktnhnnvywlt
ippmknlalgvintlewiprfkvspniftvpikeagedchaptyIpaevd
gdvkissnlvilpgqdlqyvlatydtsrvehawyyvyspsrsfsyfypfr
ipikgvpielqvecftwdkklwcrhfcvladsesgghithsgmvgmgvsc
tvtredgtnrr

The above sequences include an N-terminal signal peptide and a MuV F ectodomain in combination with various other elements, including a GCN4 trimerization domain, a T4-fibritin trimerization domain, peptide cleavage sites (e.g., thrombin), a HIS tag, a Strep tag, as well as various linker residues between segments. Purified forms of these proteins typically lack the N-terminal signal peptide and C-terminal residues removed by peptide cleavage.

### B. Recombinant MeV F Ectodomain Trimers

Recombinant MeV F ectodomain trimers are disclosed herein that are modified from a native form (e.g., by introduction of one or more amino acid substitutions) to be stabilized in a prefusion conformation. As described in the Examples, embodiments of the disclosed MeV F ectodomain trimers have been selected through multiple rounds of structure based design for optimized solubility, stability, expression, and immunogenicity. The recombinant MeV F ectodomain trimers are useful to induce an immune response in a vertebrate animal (such humans) to MeV. Exemplary embodiments are shown to produce a superior immune response in an animal model compared to corresponding MeV F ectodomain trimers that are not stabilized in the prefusion conformation.

In some embodiments, the immunogen comprises a recombinant MeV F ectodomain trimer comprising protomers comprising one or more amino acid substitutions or deletions that stabilize the MeV F ectodomain trimer in the prefusion conformation.

In some embodiments, the protomers of the recombinant MeV F ectodomain trimer comprise cysteine substitutions at MeV F positions 48 and 284 (such as R48C and A284C substitutions) that form a non-natural intra-protomer disulfide bond for stabilization in the prefusion conformation.

In some embodiments, the protomers of the recombinant MeV F ectodomain trimer comprise cysteine substitutions at MeV F positions 90 and 225 (such as A90C and 1225C substitutions) that form a non-natural intra-protomer disulfide bond for stabilization in the prefusion conformation.

In some embodiments, the protomers of the recombinant MeV F ectodomain trimer comprise cysteine substitutions at MeV F positions 141 and 270 (such as M141C and T270C substitutions) that form a non-natural intra-protomer disulfide bond for stabilization in the prefusion conformation.

In some embodiments, the protomers of the recombinant MeV F ectodomain trimer comprise cysteine substitutions at MeV F positions 165 and 171 (such as R165C and M171C substitutions) that form a non-natural intra-protomer disulfide bond for stabilization in the prefusion conformation.

In some embodiments, the protomers of the recombinant MeV F ectodomain trimer comprise cysteine substitutions at MeV F positions 173 and 245 (such as L173C and V245C substitutions) that form a non-natural intra-protomer disulfide bond for stabilization in the prefusion conformation.

In some embodiments, the protomers of the recombinant MeV F ectodomain trimer comprise cysteine substitutions at MeV F positions 175 and 241 (such as V175C and D241C substitutions) that form a non-natural intra-protomer disulfide bond for stabilization in the prefusion conformation.

In some embodiments, the protomers of the recombinant MeV F ectodomain trimer comprise cysteine substitutions at MeV F positions 212 and 236 (such as E212C and Y236C substitutions) that form a non-natural intra-protomer disulfide bond for stabilization in the prefusion conformation.

In some embodiments, the protomers of the recombinant MeV F ectodomain trimer comprise cysteine substitutions at MeV F positions 216 and 233 (such as L216C and A233C substitutions) that form a non-natural intra-protomer disulfide bond for stabilization in the prefusion conformation.

In some embodiments, the protomers of the recombinant MeV F ectodomain trimer comprise cysteine substitutions at MeV F positions 219 and 224 (such as P219C and P224C substitutions) that form a non-natural intra-protomer disulfide bond for stabilization in the prefusion conformation.

In some embodiments, the protomers of the recombinant MeV F ectodomain trimer comprise cysteine substitutions at MeV F positions 99 and 117 (such as R99C and V117C substitutions) that form a non-natural intra-protomer disulfide bond for stabilization in the prefusion conformation.

In some embodiments, the protomers of the recombinant MeV F ectodomain trimer comprise cysteine substitutions at MeV F positions 100 and 117 (such as P100C and V 117C substitutions) that form a non-natural intra-protomer disulfide bond for stabilization in the prefusion conformation.

In some embodiments, the protomers of the recombinant MeV F ectodomain trimer comprise cysteine substitutions at MeV F positions 101 and 117 (such as V101C and V 117C substitutions) that form a non-natural intra-protomer disulfide bond for stabilization in the prefusion conformation.

In some embodiments, the protomers of the recombinant MeV F ectodomain trimer comprise cysteine substitutions at MeV F positions 102 and 117 (such as Q102C and V 117C substitutions) that form a non-natural intra-protomer disulfide bond for stabilization in the prefusion conformation.

In some embodiments, the protomers of the recombinant MeV F ectodomain trimer comprise cysteine substitutions at MeV F positions 103 and 117 (such as S103C and V117C substitutions) that form a non-natural intra-protomer disulfide bond for stabilization in the prefusion conformation.

In some embodiments, the protomers of the recombinant MeV F ectodomain trimer comprise cysteine substitutions at MeV F positions 165 and 171 (such as R165C and M171C substitutions) and positions 141 and 270 (such as M141C and T270C substitutions) that form a non-natural intra-protomer disulfide bond for stabilization in the prefusion conformation.

In some embodiments, the protomers of the recombinant MeV F ectodomain trimer comprise cysteine substitutions at MeV F positions 165 and 171 (such as R165C and M171C substitutions) and positions 212 and 236 (such as E212C and Y236C substitutions) that form a non-natural intra-protomer disulfide bond for stabilization in the prefusion conformation.

In some embodiments, the protomers of the recombinant MeV F ectodomain trimer comprise cysteine substitutions at MeV F positions 165 and 171 (such as R165C and M171C substitutions) and positions 48 and 284 (such as R48C and A284C substitutions) that form a non-natural intra-protomer disulfide bond for stabilization in the prefusion conformation.

In some embodiments, the protomers of the recombinant MeV F ectodomain trimer comprise a phenylalanine at MeV F position 175 (such as a V175F substitution) for stabilization in the prefusion conformation. The phenylalanine substitution can be combined with any of the disclosed cysteine substitutions or the proline substitution at MeV F position 194 to stabilize the recombinant MeV F ectodomain trimer in the prefusion conformation.

In some embodiments, the protomers of the recombinant MeV F ectodomain trimer comprise a proline substitution at MeV F position 194 (such as a S194P substitution) for stabilization in the prefusion conformation. The proline substitution can be combined with any of the disclosed cysteine substitutions to stabilize the recombinant MeV F ectodomain trimer in the prefusion conformation.

Any of the above recombinant MeV F proteins can further comprise modification to eliminate the protease cleavage site between the F1 and F2 polypeptides to generate a “single chain” recombinant F protein. For example, any of the above recombinant MeV F proteins can comprise deletion of MeV F positions 111-113 with positions 110 and 114 fused by a peptide linker. This modification removes the F2/F1 furin cleavage site and also removed the first residue of the fusion peptide (which is hydrophobic). Any suitable peptide linker may be used that fuses the F2 and F1 ectodomain and allows folding of the F ectodomain into the prefusion conformation. In some embodiments, the peptide linker is a glycine, serine, or glycine-serine peptide linker. In some embodiments, the peptide linker is a Gly-Gly-Gly linker.

In a non-limiting example, a recombinant MeV F ectodomain trimer is provided that includes protomers with R165C and M171C substitutions to form a non-natural disulfide bond and a deletion of MeV F positions 111-113 with positions 110 and 114 fused by a Gly-Gly-Gly peptide linker.

In several embodiments, the protomers of the recombinant MeV F ectodomain can comprise one or more additional amino acid substitutions, for example, to increase stabilization of the prefusion conformation, or for other purposes, such as to increase solubility or to reduce and unwanted immune response.

The above-listed non-native disulfide bonds stabilize the membrane-distal portion of the MeV F ectodomain in its prefusion conformation. Any of these mutations can be combined with modifications to the membrane proximal portion (such as the stem) of the MeV F ectodomain, for example, to increase trimerization of the ectodomain.

In several embodiments, the N-terminal position of the recombinant F2 polypeptide in the protomer can be one of MeV F positions 24-34 (such as position 24), and the C-terminal position of the F1 ectodomain can be from the stem region of the ectodomain, such as one of MeV F positions 472-486 (such as position 486).

In a non-limiting example, a recombinant MeV F ectodomain trimer is provided that includes protomers including MeV positions 24-486 with R165C and M171C substitutions to form a non-natural disulfide bond and a deletion of MeV F positions 111-113 with positions 110 and 114 fused by a Gly-Gly-Gly peptide linker.

Non-limiting examples of protomers of a MeV F ectodomain trimer including amino acid substitutions for stabilization in the prefusion conformation are provided herein. In some embodiments, the protomers of the MeV F ectodomain trimer comprise an amino acid sequence at least 90% identical to residues 21-483 of any one of SEQ ID NOs: 37-43 or 53-55; wherein the protomers comprise the one or more amino acid substitutions that stabilize the MeV F ectodomain trimer in the prefusion conformation. In some embodiments, the protomers of the MeV F ectodomain trimer comprise residues 21-483 of any one of SEQ ID NOs: 37-43 or 53-55.

In several embodiments, the recombinant MeV F ectodomain trimer is a soluble protein complex, for example, for use as a recombinant subunit vaccine. In several such embodiments, the protomers of the recombinant MeV F ectodomain trimer can each comprise a C-terminal linkage to a trimerization domain, such as a GCN4 trimerization domain or a T4 fibritin trimerization domain, or both. The trimerization domain promotes trimerization and stabilization of the membrane proximal aspect of the recombinant MeV F ectodomain trimer. For example, a C-terminal residue of the protomers of the recombinant MeV F ectodomain trimer (such as a residue of the stem region of the trimer) can be directly linked to the trimerization domain, or indirectly linked to the trimerization domain via a peptide linker. Exemplary linkers include glycine and glycine-serine linkers. Non-limiting examples of exogenous multimerization domains that promote stable trimers of soluble recombinant proteins include: the GCN4 leucine zipper, a T4 fibritin trimerization domain, the trimerization motif from the lung surfactant protein (Hoppe et al. 1994 FEBS Lett 344:191-195) or collagen (McAlinden et al. 2003 J Biol Chem 278:42200-42207), any of which can be linked to the C-terminus of the protomers of a recombinant MeV F ectodomain to promote trimerization, as long as the recombinant MeV F ectodomain trimer retains the prefusion conformation. In some examples, the protomers of the recombinant MeV F ectodomain trimer can be linked to a MeV trimerization domain, for example, each protomer in the trimer can include a C-terminal linkage to the GCN4 trimerization domain, such as a linkage to any one of MeV F positions 472-486, such as MeV F position 486. In specific examples, the GCN4 trimerization domain comprises or consists of the amino acid sequence IEDKIEEILSKIYHIENEIARIKKLIGEAP (SEQ ID NO: 33). In specific examples, the T4 fibritin trimerization domain comprises or consists of the amino acid sequence GYIPEAPRDGQAYVRKDGEWVLLSTFL (SEQ ID NO: 34). In specific examples, the GCN4 trimerization domain fused to the fibritin trimerization domain comprises or consists of the amino acid sequence

(SEQ ID NO: 35)
IEDKIEEILSKIYHIENEIARIKKLIGEAPGSGYIPEAPRDGQAYVRKDG
EWVLLSTFL.

In a non-limiting example, a recombinant MeV F ectodomain trimer is provided that includes protomers including MeV positions 24-486 with R165C and M171C substitutions to form a non-natural disulfide bond and a deletion of MeV F positions 111-113 with positions 110 and 114 fused by a Gly-Gly-Gly peptide linker, and a GCN4 trimerization domain linked to the C-terminus of the protomers in the ectodomain.

Non-limiting examples of protomers of a MeV F ectodomain trimer including amino acid substitutions for stabilization in the prefusion conformation as well as a C-terminal linkage to a trimerization domain are provided herein. In some embodiments, the protomers of the MeV F ectodomain trimer comprise an amino acid sequence at least 90% identical to residues 21-513 of any one of SEQ ID NOs: 37-43 or 53-55; wherein the protomers comprise the one or more amino acid substitutions that stabilize the MeV F ectodomain trimer in the prefusion conformation. In some embodiments, the protomers of the MeV F ectodomain trimer comprise residues 21-513 of any one of SEQ ID NOs: 37-43 or 53-55.

In some embodiments, the recombinant MeV F ectodomain trimer can be a membrane anchored protein complex, for example, for use in an attenuated virus or virus like particle vaccine. Membrane anchoring can be accomplished, for example, by C-terminal linkage of the protomers of the recombinant MeV F ectodomain trimer to a transmembrane domain and optionally a cytoplasmic tail, such as a MeV F transmembrane domain and cytoplasmic tail. In some embodiments, one or more peptide linkers (such as a gly-ser linker, for example, a 10 amino acid glycine-serine peptide linker can be used to link the protomers of the recombinant MeV F ectodomain trimer to the transmembrane domain. A non-limiting example of a transmembrane domain for use with the disclosed embodiments includes a MeV F transmembrane domain, such as CIVYILIAVCLGGLIGI (SEQ ID NO: 100). A non-limiting example of a transmembrane domain for use with the disclosed embodiments includes a MeV F transmembrane domain, such as

(SEQ ID NO: 101)
CIVYILIAVCLGGLIGIPALICCCRGRCNKKGEQVGMSRPGLKPDLTGTS
KSYVRSL.

Native MeV F proteins from different MeV strains, as well as nucleic acid sequences encoding such proteins and methods, are known and can be altered using the description provided herein to generate a recombinant MeV F ectodomain trimer.

The recombinant MeV F ectodomain trimer can be derivatized or linked to another molecule (such as another peptide or protein). In general, the recombinant MeV F ectodomain is derivatized such that the binding to cross-neutralizing antibodies to a trimer of the recombinant MeV F protein is not affected adversely by the derivatization or labeling. For example, the recombinant MeV F ectodomain can be functionally linked (by chemical coupling, genetic fusion, non-covalent association or otherwise) to one or more other molecular entities, such as a cater protein, an antibody, a heterologous protein, or a detection tag.

In some embodiments, the recombinant MeV F ectodomain trimers are fused to one or more MuV HN ectodomains, such as the ectodomain of the MuV HN sequence set forth as:

MuV HN ectodomain head
(SEQ ID NO: 30)
NIPLVNDLRFINGINKFIIEDYATHDFSIGHPLNMPSFIPTATSPNGCTR
IPSFSLGKTHWCYTHNVINANCKDHTSSNQYVSMGILVQTASGYPMFKTL
KIQYLSDGLNRKSCSIATVPDGCAMYCYVSTQLETDDYAGSSPPTQKLTL
LFYNDTVTERTISPSGLEGNWATLVPGVGSGIYFENKLIFPAYGGVLPNS
TLGVKSAREFFRPVNPYNPCSGPQQDLDQRALRSYFPSYFSNRRIQSAFL
VCAWNQILVTNCELVVPSSNQTMMGAEGRVLLINNRLLYYQRSTSWWPYE
LLYEISFTFTNSGPSSVNMSWIPIYSFTRPGSGNCSGENVCPTACVSGVY
LDPWPLTPYSHQSGINRNFYFTGALLNSSTTRVNPTLYVSALNNLKVLAP
YGTQGLFASYTTTTCFQDTGDASVYCVYIMELASNIVGEFQILPVLTRLT
IT

For example, the protomers of the recombinant MeV F ectodomain trimer are each fused to a MuV HN ectodomain. The fusion can be direct or via a peptide linker. In some embodiments, the MuV HN ectodomain can be fused, directly or indirectly via a peptide linker, to the C-terminus of the protomers of the MeV F ectodomain trimer. In some such embodiments, the MuV HN ectodomain can be fused, directly or indirectly via a peptide linker, to the C-terminus of a trimerization domain (such as a GCN4 or T4 fibritin trimerization domain) fused to the C-terminus of the protomers of the MeV F ectodomain trimer. In some such embodiments, the protomers of the MeV F ectodomain trimer linked to the trimerization domain and the MuV HN ectodomain comprise an amino acid sequence set forth as residues 20-966 of SEQ ID NO: 27, or an amino acid sequence at least 90% identical to residues 20-966 of SEQ ID NO: 27.

In some embodiments, the recombinant MeV F ectodomain trimers are fused to one or more MeV H ectodomains, such as the ectodomain of the H sequence set forth as:

MeV H ectodomain
(SEQ ID NO: 31)
FLAVSKGNCSGPTTIRGQFSNMSLSLLDLYLGRGYNVSSIVTMTSQGMYG
GTYLVEKPNLSSKRSELSQLSMYRVFEVGVIRNPGLGAPVFHMTNYLEQP
VSNDLSNCMVALGELKLAALCHGEDSITIPYQGSGKGVSFQLVKLGVWKS
PADMQSWVPLSTDDPVIDRLYLSSHRGVIADNQAKWAVPTTRTDDKLRME
TCFQQACKGKIQTLCENPEWAPLKDNRIPSYGVLSVDLSLTVELKIKIAS
GFGPLITHGSGMDLYKSNHNNVYWLTIPPMKNLALGVINTLEWIPRFKVS
PYLFTVPIKEAGEDCHAPTYLPAEVDGDVKLSSNLVILPGQDLQYVLATY
DTSRVEHAVVYYVYSPGRSFSYFYPFRLPIKGVPIELQVECFTWDQKLWC
RHFCVLADSESGGHITHSGMVGMGVSCTVTREDGTNRR
MeV H ectodomain
(SEQ ID NO: 32)
ADVAAEELMNALVNSTLLEARATNQFLAVSKGNCSGPTTIRGQFSNMSLS
LLDLYLSRGYNVSSIVTMTSQGMYGGTYLVGKPNLSSKGSELSQLSMHRV
FEVGVIRNPGLGAPVFHMTNYFEQPVSNDFSNCMVALGELKFAALCHRED
SITIPYQGSGKGVSFQLVKLGVWKSPTDMRSWVPLSTDDPVIDRLYLSSH
RGVIADNQAKWAVPTTRTDDKLRMETCFQQACKGKNQALCENPEWAPLKD
NRIPSYGVLSVNLSLTVELKIKIASGFGPLITHGSGMDLYKTNHNNVYWL
TIPPMKNLALGVINTLEWIPRFKVSPNLFTVPIKEAGEDCHAPTYLPAEV
DGDVKLSSNLVILPGQDLQYVLATYDTSRVEHAVVYYVYSPSRSFSYFYP
FRLPIKGVPIELQVECFTWDKKLWCRHFCVLADSESGGHITHSGMVGMGV
SCTVTREDGTNRR

For example, the protomers of the recombinant MeV F ectodomain trimer are each fused to a MeV H ectodomain. The fusion can be direct or via a peptide linker. In some embodiments, the MeV H ectodomain can be fused, directly or indirectly via a peptide linker, to the C-terminus of the protomers of the MeV F ectodomain trimer. In some such embodiments, the MeV H ectodomain can be fused, directly or indirectly via a peptide linker, to the C-terminus of a trimerization domain (such as a GCN4 or T4 fibritin trimerization domain) fused to the C-terminus of the protomers of the MeV F ectodomain trimer. In some such embodiments, the protomers of the MeV F ectodomain trimer linked to the trimerization domain and the MeV H ectodomain comprise an amino acid sequence set forth as residues 21-959 of SEQ ID NO: 56 or 21-973 of SEQ ID NO 57 or an amino acid sequence at least 90% identical thereto.

Non-limiting examples of sequences containing a MeV F ectodomain with amino acid substitutions for stabilization in a prefusion conformation are provided as follows:

MeV F R48C/A284C-GGG-486-GCN4
(SEQ ID NO: 53)
mysmqlascvtltlvllvnsqihwgnlskigvvgigsasykvmtCsshqslviklmpnitllnnctrveiaeyrrllrtv
lepirdalnavtqnirpvqsvassrrhGGGagvvlagaalgvataaqitagialhqsmlnsqaidnlraslettnqaiea
irqagqemilavqgvqdyinnelipsmnqlscdligqklglkllryytellsifgpslrdpisaeisiqalsyalggdin
kvleklgysggdllgilesrgikarithvdtesyfivlsiCyptlseikgvivhrlegvsynigsqewyttvpkyvatqg
ylisnfdessctfmpegtvcsqnalypmspllqeclrgstkscartlvsgsfgnrfiisqgnllaneaslickcyttgti
inqdpdkiltyiaadhcpvvevngvtiqvgsrrypdavylhridlgppislerIdvgtnlgnaiakledakellessdqi
lsaIEDKIEEILSKIYHIENEIARIKKLIGEAP
MeV F A90C/I225C-GGG-486-GCN4
(SEQ ID NO: 37)
mysmqlascvtltlvllvnsqihwgnlskigvvgigsasykvmtrsshqslviklmpnitllnnctrveiaeyrrllrtv
lepirdClnavtqnirpvqsvassrrhGGGagvvlagaalgvataaqitagialhqsmlnsqaidnlraslettnqaiea
irqagqemilavqgvqdyinnelipsmnqlscdligqklglkllryytellsifgpslrdpCsaeisiqalsyalggdin
kvleklqysqqdllqilesrgikarithvdtesyfivlsiayptlseikqvivhrleqvsynigsqewyttvpkyvatqq
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F M141C/T270C-GGG-486-GCN4
(SEQ ID NO: 54)
mysmqlascvtltlvllvnsqihwqnlskigvvgiqsasykvmtrsshqslviklmpnitllnnctrveiaeyrrllrtv
lepirdalnavtqnirpvqsvassrrhGGGagvvlagaalgvataaqitagialhqsClnsqaidnlraslettnqaiea
irqagqemilavqgvqdyinnelipsmnqlscdligqklglkllryytellsifgpslrdpisaeisiqalsyalggdin
kvleklgysggdllgilesrgikariChvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F R165C/M171C-GGG-486-GCN4
(SEQ ID NO: 38)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaaIgvataaqitagiaihqsmlnsqaidnlraslettnqaiea
iCqagqeCilavqgvqdyinnelipsmnqlscdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F L173C/V245C-GGG-486-GCN4
(SEQ ID NO: 39)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaaigvataaqitagiaihqsmlnsqaidnlraslettnqaiea
irqagqemiCavqgvqdyinnelipsmnqlscdligqklglkllryyteilsifgpslrdpisaeisiqalsyalggdin
kCleklqysqqdllqilesrqikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F V175C/D241C-GGG-486-GCN4
(SEQ ID NO: 40)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaaigvataaqitagiaihqsmlnsqaidnlraslettnqaiea
irqagqemilaCqgvqdyinnelipsmnqlscdligqklglkllryyteilslfgpslrdpisaeisiqalsyalggCin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F E212C/Y236C-GGG-486-GCN4
(SEQ ID NO: 55)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaaigvataaqitagiaihqsmlnsqaidnlraslettnqaiea
irqagqemilavqgvqdyinnelipsmnqlscdligqklglkllryytCilslfgpslrdpisaeisiqalsCalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F L216C/A233C-GGG-486-GCN4
(SEQ ID NO: 41)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaaigvataaqitagiaihqsmlnsqaidnlraslettnqaiea
irqagqemilavqgvqdyinnelipsmnqlscdligqklglkllryyteilsCfgpslrdpisaeisiqClsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F P219C/P224C-GGG-486-GCN4
(SEQ ID NO: 42)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaaigvataaqitagiaihqsmlnsqaidnlraslettnqaiea
irqagqemilavqgvqdyinnelipsmnqlscdligqklglkllryyteilslfgCslrdCisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerIdvgtnlgnaiakledakellessdqi
lsaIEDKIEEILSKIYHIENEIARIKKLIGEAP
MeV F S194P-GGG-486-GCN4
(SEQ ID NO: 43)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaalgvataaqitagialhqsmlnsqaidnlraslettnqaiea
irqagqemilavqgvqdyinnelipsmnqlPcdligqklglkllryyteilslfgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerIdvgtnlgnaiakledakellessdqi
lsaIEDKIEEILSKIYHIENEIARIKKLIGEAP
MeV F S103C/V117C-GGG-486-GCN4
(SEQ ID NO: 62)
mysmqlascvtltlvllvnsqihwqnlskigvvgiqsasykvmtrsshqslviklmpnitllnnctrveiaeyrrllrtv
lepirdalnavtqnirpvqCvassrrhGGGagvClagaaIgvataaqitagiaIhqsmlnsqaidnlraslettnqalea
irqagqemilavqgvqdyinnelipsmnqlscdligqklglkllryytellsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerIdvgtnlgnaiakledakellessdqi
lsaIEDKIEEILSKIYHIENEIARIKKLIGEAP
MeV F V175F-GGG-486-GCN4
(SEQ ID NO: 63)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaaigvataaqitagiaihqsmlnsqaidnlraslettnqalea
irqagqemilaFqgvqdyinnelipsmnqlscdligqklglkllryytellsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerIdvgtnlgnaiakledakellessdqi
lsaIEDKIEEILSKIYHIENEIARIKKLIGEAP
MeV F S103C/V117C, V175F, S194P-GGG-486-GCN4
(SEQ ID NO: 64)
mysmqlascvtitlvllvnsqihwgnlskigvvgigsasykvmtrsshqslviklmpnitllnnctrveiaeyrrllrtv
lepirdalnavtqnirpvqCvassrrhGGGagvClagaaigvataaqitagiaihqsmlnsqaidnlraslettnqaiea
irqagqemilaFqgvqdyinnelipsmnqlPcdligqklglkllryytellsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerIdvgtnlgnaiakledakellessdqi
lsaIEDKIEEILSKIYHIENEIARIKKLIGEAP
MeV F S103C/V117C, R165C/M171C, S194P-GGG-486-GCN4
(SEQ ID NO: 65)
mysmqlascvtltlvllvnsqihwgnlskigvvgigsasykvmtrsshqslviklmpnitllnnctrveiaeyrrllrtv
lepirdalnavtqnirpvqCvassrrhGGGagvClagaaIgvataaqitagiaIhqsmlnsqaidnlraslettnqalea
iCqagqeCilaFqgvqdyinnelipsmnqlPcdligqklglkllryytellsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerIdvgtnlgnaiakledakellessdqi
lsaIEDKIEEILSKIYHIENEIARIKKLIGEAP
MeV F R99C/V117C-GGG-486-GCN4
(SEQ ID NO: 66)
mysmqlascvtitlvllvnsqihwqnlskigvvgiqsasykvmtrsshqslviklmpnitllnnctrveiaeyrrllrtv
lepirdalnavtqniCpvqsvassrrhGGGagvClagaaIgvataaqitagiaihqsmlnsqaidnlraslettnqalea
irqagqemilavqgvqdyinnelipsmnqlscdligqklglkllryvte11sifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerIdvgtnlgnaiakledakellessdqi
lsaIEDKIEEILSKIYHIENEIARIKKLIGEAP
MeV F R99C/V117C, V175F, S194P-GGG-486-GCN4
(SEQ ID NO: 67)
mysmqlascvtltlvllvnsqihwgnlskigvvgigsasykvmtrsshqslviklmpnitllnnctrveiaeyrrllrtv
lepirdalnavtqniCpvqsvassrrhGGGagvClagaaigvataaqitagiaihqsminsqaidnlraslettnqaiea
irqagqemilaFqgvqdyinnelipsmnqlPcdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F R99C/VH7C, R165C/M171C, S194P-GGG-486-GCN4
(SEQ ID NO: 68)
mysmqlascvtitlvllvnsqihwgnlskigvvgigsasykvmtrsshqslviklmpnitllnnctrveiaeyrrllrtv
lepirdalnavtqniCpvqsvassrrhGGGagvClagaaIgvataaqitagiaihqsminsqaidnlraslettnqaiea
iCqagqeCilaFqgvqdyinnelipsmnqlPcdligqklglkllryytellsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F R99C/V117C-GGG-493-GCN4
(SEQ ID NO: 69)
mysmqlascvtltlvllvnsqihwgnlskigvvgigsasykvmtrsshqslviklmpnitllnnctrveiaeyrrllrtv
lepirdalnavtqniCpvqsvassrrhGGGagvClagaaigvataaqitagiaihqsminsqaidnlraslettnqaiea
irqagqemilavqgvqdvinnelipsmnqlscdligqklglkllrvvteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lrsmkglssaIEDKIEEILSKIYHIENEIARIKKLIGEAP
MeV F R99C/V117C, V175F, S194P-GGG-493-GCN4
(SEQ ID NO: 70)
mysmqlascvtltlvllvnsqihwgnlskigvvgigsasykvmtrsshqslviklmpnitllnnctrveiaeyrrllrtv
lepirdalnavtqniCpvqsvassrrhGGGagvClagaaIgvataaqitagiaihqsminsqaidnlraslettnqaiea
irqagqemilaFqgvqdyinnelipsmnqlPcdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lrsmkglssaIEDKIEEILSKIYHIENEIARIKKLIGEAP
MeV F R99C/V117C, R165C/M171C, S194P-GGG-493-GCN4
(SEQ ID NO: 71)
mysmqlascvtltlvllvnsqihwgnlskigvvgigsasykvmtrsshqslviklmpnitllnnctrveiaeyrrllrtv
lepirdalnavtqniCpvqsvassrrhGGGagvClagaaIgvataaqitagiaihqsminsqaidnlraslettnqaiea
iCqagqeCilaFqgvqdyinnelipsmnqlPcdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lrsmkglssaIEDKIEEILSKIYHIENEIARIKKLIGEAP
MeV F P100C/V117C, R165C/M171C, S194P-GGG-486-GCN4
(SEQ ID NO: 72)
mysmqlascvtltlvllvnsqihwgnlskigvvgigsasykvmtrsshqslviklmpnitllnnctrveiaeyrrllrtv
lepirdaInavtqnirCvqsvassrrhGGGagvClagaaigvataaqitagiaihqsminsqaidnlraslettnqaiea
iCqagqeCilaFqgvqdyinnelipsmnqlPcdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F V101C/V117C, R165C/M171C, S194P-GGG-486-GCN4
(SEQ ID NO: 73)
mysmqlascvtltlvllvnsqihwgnlskigvvgigsasykvmtrsshqslviklmpnitllnnctrveiaeyrrllrtv
lepirdaInavtqnirpCqsvassrrhGGGagvClagaaigvataaqitagiaihqsminsqaidnlraslettnqaiea
iCqagqeCilaFqgvqdyinnelipsmnqlPcdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F Q102C/V117C, R165C/M171C, S194P-GGG-486-GCN4
(SEQ ID NO: 74)
mysmqlascvtltlvllvnsqihwqnlskigvvgiqsasykvmtrsshqslviklmpnitllnnctrveiaeyrrllrtv
lepirdalnavtqnirpvCsvassrrhGGGagvClagaaigvataaqitagiaihqsmlnsqaidnlraslettnqaiea
iCqagqeCilaFqgvqdyinnelipsmnqlPcdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerIdvgtnlgnaiakledakellessdqi
lsaIEDKIEEILSKIYHIENEIARIKKLIGEAP
MeV F R165C/M171C, Ml 41C/T270C-GGG-486-GCN4
(SEQ ID NO: 75)
mysmqlascvtitlvllvnsqihwgnlskigvvgigsasykvmtrsshqslviklmpnitllnnctrveiaeyrrllrtv
lepirdalnavtqnirpvqsvassrrhGGGagvvlagaaigvataaqitagiaihqsClnsqaidnlraslettnqaiea
iCqagqeCilavqgvqdyinnelipsmnqlscdligqklglkllryyteilsifgpslrdpisaeisiqalsyalggdin
kvleklgysggdllgilesrgikarichvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F R165C/M171C, E212C/Y236C-GGG-486-GCN4
(SEQ ID NO: 76)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaaigvataaqitagiaihqsmlnsqaidnlraslettnqaiea
iCqagqeCilavqgvqdyinnelipsmnqlscdligqklglkllryytCilslfgpslrdpisaeisiqalsCalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F R165C/M171C, R48C/A284C -GGG-486-GCN4
(SEQ ID NO: 77)
mysmqlascvtltlvllvnsqihwgnlskigvvgigsasykvmtCsshqslviklmpnitllnnctrveiaeyrrllrtv
lepirdalnavtqnirpvqsvassrrhGGGagvvlagaaigvataaqitagiaihqsmlnsqaidnlraslettnqaiea
iCqagqeCilavqgvqdyinnelipsmnqlscdligqklglkllryyteilsifgpslrdpisaeisiqalsyalggdin
kvleklgysggdllgilesrgikarithvdtesyfivisiCyptIseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F R99C/V117C, native furin-486-GCN4
(SEQ ID NO: 78)
mysmqlascvtltlvllvnsqihwgnlskigvvgigsasykvmtrsshqslviklmpnitllnnctrveiaeyrrllrtv
lepirdaInavtqniCpvqsvassrrhkrfagvClagaaigvataaqitagiaihqsmlnsqaidnlraslettnqaiea
irqagqemilavqgvqdyinnelipsmnqlscdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F R99C/V117C, V175F, S194P, native furin-486-GCN4
(SEQ ID NO: 79)
mysmqlascvtltlvllvnsqihwgnlskigvvgigsasykvmtrsshqslviklmpnitllnnctrveiaeyrrllrtv
lepirdaInavtqniCpvqsvassrrhkrfagvClagaaigvataaqitagiaihqsmlnsqaidnlraslettnqaiea
irqagqemilaFqgvqdyinnelipsmnqlPcdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F R99C/V117C, R165C/M171C, S194P, native furin-486-GCN4
(SEQ ID NO: 80)
mysmqlascvtltlvllvnsqihwgnlskigvvgigsasykvmtrsshqslviklmpnitllnnctrveiaeyrrllrtv
lepirdaInavtqniCpvqsvassrrhkrfagvClagaaigvataaqitagiaihqsmlnsqaidnlraslettnqaiea
iCqagqeCilaFqgvqdyinnelipsmnqlPcdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP

The above sequences include an N-terminal signal peptide, a MeV F ectodomain, and a GCN4 trimerization domain. It will be appreciated that an alternative trimerization domain can be used, such as a T4 Fibritin trimerization domain. Additionally, many of the above sequence include a GGG linker to remove the native furin cleavage site separating the F1 and F2 subunits. Alternate glycine linkers can also be used, such as GSG, GGS, or SGG. Additionally, in any of the sequences, the native furin cleavage site can be included instead of the GGG linker. It will be appreciated that the N-terminal signal peptide is removed during cellular processing and is not present in the purified protein. Additionally, the MeV F ectodomain of any of the above sequences can be included in a full-length MeV F protein to provide a membrane anchored version of the prefusion MeV F protein, for example for mRNA immunization.

Non-limiting examples of sequences containing a MeV F ectodomain with amino acid substitutions for stabilization in a prefusion conformation linked to a MuV HN ectodomain or a MeV H ectodomain are provided as follows:

MeV F R165C/M171C-GGG-486-GCN4 + MeV_H
(SEQ ID NO: 56)
mysmqlascvtltlvllvnsqihwgnlskigvvgigsasykvmtrsshqs
lviklmpnitllnnctrveiaeyrrllrtvlepirdalnavtqnirpvqs
vassrrhGGGagvvlagaalgvataaqitagialhqsmlnsqaidnlras
lettnqaieaiCqagqeCilavqgvqdyinnelipsmnqlscdligqklg
lkllryyteilslfgpslrdpisaeisiqalsyalggdinkvleklgysg
gdllgilesrgikarithvdtesyfivlsiayptlseikgvivhrlegvs
ynigsqewyttvpkyvatqgylisnfdessctfmpegtvcsqnalypmsp
llqeclrgstkscartlvsgsfgnrfilsqgnliancasilckcyttgti
inqdpdkiltyiaadhcpvvevngvtiqvgsrrypdavylhridlgppis
leridvgtnlgnaiakledakellessdqilsaIEDKIEEILSKIYHIEN
EIARIKKLIGEAPGSGGGGGGflavskgncsgpttirgqfsnmsislldl
ylgrgynvssivtmtsqgmyggtyivekpnlsskrselsqlsmyrvfevg
virnpglgapvfhmtnyleqpvsndlsncmvalgelklaalchgedsiti
pyqgsgkgvsfqlvklgvwkspadmqswvplstddpvidrlyisshrgvi
adnqakwavpttrtddklrmetcfqqackgkiqticenpewaplkdnrip
sygvlsvdlsltvelkikiasgfgplithgsgmdlyksnhnnvywltipp
mknlalgvintlewiprfkvspylftvpikeagedchaptyipaevdgdv
klssnlvilpgqdlqyvlatydtsrvehavvyyvyspgrsfsyfypfrip
ikgvpielqvecftwdqklwcrhfcvladsesgghithsgmvgmgvsctv
tredgtnrr
MeV F R165C/M171C-GGG-486-GCN4 + MuV HN
(SEQ ID NO: 57)
mysmqlascvtltlvllvnsqihwqnlskiqvvqiqsasykvmtrsshqs
lviklmpnitllnnctrveiaeyrrHrtvlepirdalnavtqnirpvqsv
assrrhGGGagvvlagaalgvataaqitagiaihqsmlnsqaidnlrasl
ettnqaieaiCqagqeCilavqgvqdyinnelipsmnqlscdligqklgl
kllryyteilsifgpslrdpisaeisiqalsyalggdinkvleklgysgg
dllgilesrgikarithvdtesyfivlsiayptlseikgvivhrlegvsy
nigsqewyttvpkyvatqgylisnfdessctfmpegtvcsqnalypmspl
lqeclrgstkscartlvsgsfgnrfilsqgnliancasilckcyttgtii
nqdpdkiltyiaadhcpvvevngvtiqvgsrrypdavylhridlgppisl
eridvgtnlgnaiakledakellessdqilsaIEDKIEEILSKIYHIENE
IARIKKLIGEAPGsggggggniplvndlrfinginkfHedyathdfsigh
plnmpsfiptatspngctripsfslgkthwcythnvinanckdhtssnqy
vsmqilvqtasgypmfktikiqylsdglnrkscsiatvpdgcamycyvst
qletddyagsspptqkltllfyndtvtertispsglegnwativpgvgsg
iyfenklifpayggvlpnstigvksareffrpvnpynpcsgpqqdldqra
lrsyfpsyfsnrriqsaflvcawnqilvtncelvvpssnqtmmgaegrvl
linnrllyyqrstswwpyellyeisftftnsgpssvnmswipiysftrpg
sgncsgenvcptacvsgvyldpwpltpyshqsginrnfyftgallnsstt
rvnptlyvsalnnlkvlapygtqglfasyttttcfqdtgdasvycvyime
lasnivgefqilpvltritit
MeV F R165C/M171C-GGG-486-GCN4/Fd + MeV_H
(SEQ ID NO: 81)
mysmqlascvtltlvllvnsqihwgnlskigvvgigsasykvmtrsshqs
lviklmpnitllnnctrveiaeyrrllrtvlepirdalnavtqnirpvqs
vassrrhGGGagvvlagaaigvataaqitagiaihqsmlnsqaidnlras
lettnqaieaiCqagqeCilavqgvqdyinnelipsmnqlscdligqklg
lkllryyteilsifgpslrdpisaeisiqalsyalggdinkvleklgysg
gdllgilesrgikarithvdtesyfivlsiayptlseikgvivhrlegvs
ynigsqewyttvpkyvatqgylisnfdessctfmpegtvcsqnalypmsp
llqeclrgstkscartlvsgsfgnrfilsqgnliancasilckcyttgti
inqdpdkiltyiaadhcpvvevngvtiqvgsrrypdavylhridlgppis
leridvgtnlgnaiakledakellessdqilsaIEDKIEEILSKIYHIEN
EIARIKKLIGEAPGSGYIPEAPRDGQAYVRKDGEWVLLSTFLGSGGGGGg
flavskgncsgpttirgqfsnmslslldlylgrgynvssivtmtsqgmyg
gtylvekpnlsskrselsqlsmyrvfevgvirnpglgapvfhmtnyleqp
vsndlsncmvalgelklaalchgedsitipyqgsgkgvsfqlvklgvwks
padmqswvplstddpvidrlylsshrgviadnqakwavpttrtddklrme
tcfqqackgkiqticenpewaplkdnripsygvlsvdlsitvelkikias
gfgplithgsgmdlyksnhnnvywltippmknlalgvintlewiprfkvs
pyiftvpikeagedchaptylpaevdgdvklssnlvilpgqdlqyvlaty
dtsrvehavvyyvyspgrsfsyfypfripikgvpielqvecftwdqklwc
rhfcvladsesgghithsgmvgmgvsctvtredgtnrr

The above sequences include an N-terminal signal peptide, a MeV F ectodomain, a trimerization domain (GCN4 and/or T4Fibritin), and a MeV H ectodomain head or a MuV HN ectodomain head region. The MeV F ectodomain sequence includes a GGG linker to remove the native furin cleavage site separating the F1 and F2 subunits. Alternate glycine linkers can also be used, such as GSG, GGS, or SGG. Additionally, in any of the sequences, the native furin cleavage site can be included instead of the GGG linker. It will be appreciated that the N-terminal signal peptide is removed during cellular processing and is not present in the purified protein.

### C. MuV HN and MuV H Multimers

In some embodiments, an immunogen is provided that comprises a multimer of the MuV HN ectodomain and/or the MeV H ectodomain. The H or HN ectodomain can include the ectodomain head or the ectodomain stalk and head.

In some embodiments, the immunogen comprises a trimer of fusion proteins, each fusion protein comprising one or more MuV HN ectodomains or MeV H ectodomains and a trimerization domain (such as a GCN4 trimerization domain, a T4 fibritin trimerization domain, or a GCN4 trimerization domain fused to a T4 fibritin trimerization domain).

In some embodiments, the fusion protein comprises, in an N- to C-terminal direction, a trimerization domain (such as a GCN4 trimerization domain, a T4 fibritin trimerization domain, or a GCN4 trimerization domain fused to a T4 fibritin trimerization domain) and one or more (such as one, two, or three) MuV HN ectodomains or Mev H ectodomains. The trimerization domains interact to form the trimer. In some embodiments, a fragment of the ectodomain is included, for example the head region of the MuV HN ectodomain or the head region of the MeV H ectodomain can be fused to the trimerization domain, optionally by a peptide linker. In some embodiment, the fusion proteins in the trimer comprise or consist of an amino acid sequence set forth as residues 24-510 of SEQ ID NO: 58 or residues 24-496 of SEQ ID NO: 59, or a sequence at least 90% identical to any one of residues 25-510 of SEQ ID NO: 58 or residues 25-496 of SEQ ID NO: 59.

In some embodiments, the fusion protein comprises, in an N- to C-terminal direction, one or more (such as one, two, or three) MuV HN ectodomains or Mev H ectodomains, a trimerization domain (such as a GCN4 trimerization domain, a T4 fibritin trimerization domain, or a GCN4 trimerization domain fused to a T4 fibritin trimerization domain), and one or more (such as one, two, or three) MuV HN ectodomains or Mev H ectodomains. The trimerization domains interact to form the trimer. In some embodiments, a fragment of the ectodomain is included, for example the head region of the MuV HN ectodomain or the head region of the MeV H ectodomain can be fused to the trimerization domain, optionally by a peptide linker. In some embodiment, the fusion proteins in the trimer comprise or consist of an amino acid sequence set forth as residues 22-950 of SEQ ID NO: 82, residues 25-985 of SEQ ID NO: 83, residues 22-948 of SEQ ID NO: 84, or residues 22-981 of SEQ ID NO: 85, or a sequence at least 90% identical to any one of residues 22-950 of SEQ ID NO: 82, residues 25-985 of SEQ ID NO: 83, residues 22-948 of SEQ ID NO: 84, or residues 22-981 of SEQ ID NO: 85.

In some embodiments, the multimer is a dimer of MeV H ectodomain head regions. The MeV H ectodomain head region can be expressed in mammalian cells and spontaneously forms a dimer in physiological solution. This dimer can then be purified and used as an immunogen. In some embodiments, the subunits of the dimer comprise or consist of an amino acid sequence set forth as residues 22-459 of SEQ ID NO: 60 or a sequence at least 90% identical to residues 22-459 of SEQ ID NO: 60.

In some embodiments, the multimer is a dimer of MeV H ectodomain stalk and head regions. The MeV H ectodomain stalk and head region can be expressed in mammalian cells and spontaneously form a dimer in physiological solution. This dimer can then be purified and used as an immunogen. In some embodiments, the subunits of the dimer comprise or consist of an amino acid sequence of any one of MeV H positions 59-197 to MeV H position 617 (such as any one of MeV H positions 59-67 to MeV H position 617, for example positions 59-617, 62-617, 60-617, or 67-617). In some embodiments, the subunits of the dimer comprise or consist of an amino acid sequence set forth as residues 22-580 of SEQ ID NO: 86, residues 22-577 of SEQ ID NO: 87, residues 22-579 of SEQ ID NO: 88, or residues 22-572 of SEQ ID NO: 89, or a sequence at least 90% identical to residues 22-580 of SEQ ID NO: 86, residues 22-577 of SEQ ID NO: 87, residues 22-579 of SEQ ID NO: 88, or residues 22-572 of SEQ ID NO: 89.

In some embodiments, the multimer is a dimer of MuV HN ectodomain head regions. In some embodiments, the subunits of the dimer comprise or consist of an amino acid sequence set forth as SEQ ID NO: 30 or a sequence at least 90% identical to SEQ ID NO: 30.

In some embodiments, the multimer is a dimer of MuV H ectodomain stalk and head regions. In some embodiments, the subunits of the dimer comprise or consist of an amino acid sequence of any one of MuV H positions 54-130 to MuV HN position 582 (such as any one of MuV H positions 54-63 to MuV HN position 582, for example positions 54-582, 61-582, 63-582, or 55-582). In some embodiments, the subunits of the dimer comprise or consist of an amino acid sequence set forth as residues 22-550 of SEQ ID NO: 90, residues 22-543 of SEQ ID NO: 91, residues 22-541 of SEQ ID NO: 92, or residues 22-549 of SEQ ID NO: 93, or a sequence at least 90% identical to residues 22-550 of SEQ ID NO: 90, residues 22-543 of SEQ ID NO: 91, residues 22-541 of SEQ ID NO: 92, or residues 22-549 of SEQ ID NO: 93.

### D. Additional Description

The protomers in the recombinant MuV or MeV F ectodomain trimer can comprise modifications of the native MuV F or MeV F sequence in addition to those noted above, such as amino acid substitutions, deletions or insertions, glycosylation and/or covalent linkage to unrelated proteins (e.g., a protein tag), as long as the recombinant MuV or MeV F ectodomain trimer remains stabilized in the prefusion conformation and retains immunogenicity. Further, in embodiments including a heterologous MuV HN ectodomain or a MeV H ectodomain, or a multimer of a MuV HN ectodomain or a MeV H ectodomain, the HN or H ectodomain can include modifications of the native HN or H sequence, such as amino acid substitutions, deletions or insertions, glycosylation and/or covalent linkage to unrelated proteins (e.g., a protein tag), as long as the HN or H ectodomain retains immunogenicity. These variations in sequence can be naturally occurring variations or they can be engineered through the use of genetic engineering technique known to those skilled in the art. Examples of such techniques are found in see, e.g., Sambrook et al. (Molecular Cloning: A Laboratory Manual, 4^{th} ed., Cold Spring Harbor, New York, 2012) and Ausubel et al. (In Current Protocols in Molecular Biology, John Wiley & Sons, New York, through supplement 104, 2013, both of which are incorporated herein by reference in their entirety.

In some embodiments, the protomers in the recombinant MuV F ectodomain trimer or comprise one or more amino acid substitutions compared to a corresponding native MuV F sequence. For example, in some embodiments, the F₂ polypeptide, F₁ ectodomain, or both, can include up to 20 (such as up to 1, 2, 3, 4, 5, 6, 7, 8, 9, 10, 11, 12, 13, 14, 15, 16, 17, 18, or 19) amino acid substitutions (such as conservative amino acid substitutions) compared to a native MuV F sequence.

In some embodiments, the protomers in the recombinant MeV F ectodomain trimer or comprise one or more amino acid substitutions compared to a corresponding native MeV F sequence. For example, in some embodiments, the F₂ polypeptide, F₁ ectodomain, or both, can include up to 20 (such as up to 1, 2, 3, 4, 5, 6, 7, 8, 9, 10, 11, 12, 13, 14, 15, 16, 17, 18, or 19) amino acid substitutions (such as conservative amino acid substitutions) compared to a native MeV F sequence.

In some embodiments, the MuV HN ectodomain comprises one or more amino acid substitutions compared to a corresponding native MuV HN ectodomain sequence. For example, in some embodiments, the MuV HN ectodomain includes up to 20 (such as up to 1, 2, 3, 4, 5, 6, 7, 8, 9, 10, 11, 12, 13, 14, 15, 16, 17, 18, or 19) amino acid substitutions (such as conservative amino acid substitutions) compared to a native MuV HN ectodomain sequence.

In some embodiments, the MeV H ectodomain comprises one or more amino acid substitutions compared to a corresponding native MeV H ectodomain sequence. For example, in some embodiments, the MeV H ectodomain includes up to 20 (such as up to 1, 2, 3, 4, 5, 6, 7, 8, 9, 10, 11, 12, 13, 14, 15, 16, 17, 18, or 19) amino acid substitutions (such as conservative amino acid substitutions) compared to a native MeV H ectodomain sequence.

The simplest modifications involve the substitution of one or more amino acids for amino acids having similar biochemical properties, such as conservative amino acid substitutions. Such substitutions are likely to have minimal impact on the activity of the resultant protein.

In some embodiments, protomers in the recombinant MeV F ectodomain trimer or the MuV F ectodomain trimer can be joined at either end to other unrelated sequences (for example non-MuV F or MeV F protein sequences, non-viral envelope, or non-viral protein sequences) In several embodiments, the recombinant MuV F ectodomain trimer or MeV F ectodomain trimer, or fusions of these protomers with heterologous proteins such as MuV HN ectodomain or MeV H ectodomain is soluble in aqueous solution. In some embodiments, the recombinant MuV F ectodomain trimer MeV F ectodomain trimer, or corresponding fusions with heterologous proteins such as MuV HN ectodomain or MeV H ectodomain dissolves to a concentration of at least 0.5 mg/ml (such as at least 1.0 mg/ml, 1.5 mg/ml, 2.0 mg/ml, 3.0 mg/ml, 4.0 mg/ml or at least 5.0 mg/ml) in aqueous solution (such as phosphate buffered saline (pH 7.4) or 350 mM NaCl (pH 7.0)) at room temperature (e.g., 20-22 degrees Celsius) and remain dissolved for at least 12 hours (such as at least 24 hours, at least 48 hours, at least one week, at least two weeks, at least one month, or more time). In one embodiment, the phosphate buffered saline includes NaCl (137 mM), KCl (2.7 mM), Na₂HPO₄ (10 mM), KH₂PO₄ (1.8 mM) at pH 7.4. In some embodiments, the phosphate buffered saline further includes CaCl₂) (1 mM) and MgCl₂ (0.5 mM). The person of skill in the art is familiar with methods of determining if a protein remains in solution over time. For example, the concentration of the protein dissolved in an aqueous solution can be tested over time using standard methods.

In some embodiments, the immunogen is provided as a homogenous population of soluble trimers that are substantially in the prefusion conformation with limited to no MuV F ectodomain trimer and/or MeV F ectodomain trimer in a postfusion conformation. The conformation of the MeV F ectodomain trimer or the MuV F ectodomain trimer can be detected, for example, by negative stain electron microscopy and/or specific binding by appropriate pre- or post-fusion specific antibody. In some embodiments, at least about 95% of the recombinant MuV F ectodomain trimer or MeV F ectodomain trimer (such as at least about 95%, 96%, 97%, 98%, 99% or 99.9% of the MuV or MeV F proteins) in the homogeneous population are stabilized in the prefusion conformation.

In some embodiments, the recombinant MuV F ectodomain trimer or MeV F ectodomain trimer retains specific binding for a prefusion specific antibody following incubation at 50° C. for one hour in phosphate buffered saline. In some embodiments, the recombinant MuV F ectodomain trimer or MeV F ectodomain trimer retains specific binding for a prefusion specific antibody following incubation at 4° C. for six months in phosphate buffered saline.

In certain embodiments, an immunogen provided herein may be further modified to contain additional nonproteinaceous moieties that are known in the art and readily available. The moieties suitable for derivatization of the immunogen include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the immunogen to be improved or altered, whether the immunogen derivative will be used in a therapy under defined conditions, etc.

Some of the sequences including recombinant MuV F ectodomain trimer, or MeV F ectodomain trimer, or MuV HN ectodomain or MeV H ectodomain provided herein include the sequence of protease cleavage sites (such as thrombin sites), protein tags (such as a His tag, a Strep Tag II, a Avi tag, etc.), and signal peptides; such sequences can be removed from an isolated immunogen including a recombinant MuV F ectodomain trimer, or MeV F ectodomain trimer, or MuV HN ectodomain or MeV H ectodomain for therapeutic use.

### E. Protein Nanoparticles

In some embodiments, a protein nanoparticle is provided that includes one or more of the disclosed recombinant MuV F ectodomain trimers or recombinant MeV F ectodomain trimers, or MuV HN, or MeV H multimers, or chimera thereof.

In some embodiments, the protein nanoparticle comprises the MeV F ectodomain trimer or MuV F ectodomain trimer displayed on a two-component self-assembling nanoparticle platform as described in Marcandalli et al. “Induction of potent neutralizing antibody responses by a designed protein nanoparticle vaccine for respiratory syncytial virus,” Cell, 176(6):1420-1431, 2019, which is incorporated by reference herein.

In additional non-limiting example of nanoparticles include ferritin nanoparticles, encapsulin nanoparticles, Sulfur Oxygenase Reductase (SOR) nanoparticles, and lumazine synthase nanoparticles, which are comprised of an assembly of monomeric subunits including ferritin proteins, encapsulin proteins, SOR proteins, and lumazine synthase, respectively. To construct such protein nanoparticles a protomer of the recombinant MuV F ectodomain trimer or recombinant MeV F ectodomain trimer, or a subunit of a MuV HN or MeV H multimer, is linked to a subunit of the protein nanoparticle (such as a ferritin protein, an encapsulin protein, a SOR protein, or a lumazine synthase protein) and expressed in cells under appropriate conditions. The fusion protein self-assembles into a nanoparticle any can be purified.

In some embodiments, a protomer of a disclosed recombinant MuV F ectodomain trimer or recombinant MeV F ectodomain trimer, or a subunit of a MuV HN or MeV H multimer, can be linked to a ferritin subunit to construct a ferritin nanoparticle. Ferritin nanoparticles and their use for immunization purposes (e.g., for immunization against influenza antigens) have been disclosed in the art (see, e.g., Kanekiyo et al., Nature, 499:102-106, 2013, incorporated by reference herein in its entirety). The globular form of the ferritin nanoparticle is made up of monomeric subunits, which are polypeptides having a molecule weight of approximately 17-20 kDa. Following production, these monomeric subunit proteins self-assemble into the globular ferritin protein. Thus, the globular form of ferritin comprises 24 monomeric, subunit proteins, and has a capsid-like structure having 432 symmetry. Methods of constructing ferritin nanoparticles are further described herein (see, e.g., Zhang, Int. J. Mol. Sci., 12:5406-5421, 2011, which is incorporated herein by reference in its entirety). An example of the amino acid sequence of one such monomeric ferritin subunit is represented by:

(SEQ ID NO: 45)
DIIKLLNEQVNKEMQSSNLYMSMSSWCYTHSLDGAGLFLFDHAAEEYEHA
KKLIIFLNENNVPVQLTSISAPEHKFEGLTQIFQKAYEHEQHISESINNI
VDHAIKSKDHATFNFLQWYVAEQHEEEVLFKDILDKIELIGNENHGLYLA
DQYVKGIAKSRKS

In specific examples, the ferritin polypeptide is E. coli ferritin, Helicobacter pylori ferritin, human light chain ferritin, bullfrog ferritin or a hybrid thereof, such as E. coli-human hybrid ferritin, E. coli-bullfrog hybrid ferritin, or human-bullfrog hybrid ferritin. Exemplary amino acid sequences of ferritin polypeptides and nucleic acid sequences encoding ferritin polypeptides for use to make a ferritin nanoparticle including a recombinant MuV or MeV F ectodomain trimer can be found in GENBANK®, for example at accession numbers ZP_03085328, ZP_06990637, EJB64322.1, AAA35832, NP_000137 AAA49532, AAA49525, AAA49524 and AAA49523, which are specifically incorporated by reference herein in their entirety as available Apr. 10, 2015. In some embodiments, a protomer of a recombinant MuV or MeV F ectodomain trimer can be linked to a ferritin subunit including an amino acid sequence at least 80% (such as at least 85%, at least 90%, at least 95%, or at least 97%) identical to amino acid sequence set forth as SEQ ID NO: 45.

In some embodiments, a protomer of a disclosed recombinant MuV F ectodomain trimer or recombinant MuV or MeV F ectodomain trimer, or a subunit of a MuV HN or MeV H multimer, can be linked to a lumazine synthase subunit to construct a lumazine synthase nanoparticle. The globular form of lumazine synthase nanoparticle is made up of monomeric subunits; an example of the sequence of one such lumazine synthase subunit is provides as the amino acid sequence set forth as:

(SEQ ID NO: 46)
MQIYEGKLTAEGLRFGIVASRFNHALVDRLVEGAIDCIVRHGGREEDITL
VRVPGSWEIPVAAGELARKEDIDAVIAIGVLIRGATPHFDYIASEVSKGL
ADLSLELRKPITFGVITADTLEQAIERAGTKHGNKGWEAALSAIEMANLF
KSLR.

In some embodiments, a protomer of a disclosed recombinant MuV F ectodomain trimer or recombinant MeV F ectodomain trimer, or a subunit of a MuV HN or MeV H multimer, can be linked to a lumazine synthase subunit including an amino acid sequence at least 80% (such as at least 85%, at least 90%, at least 95%, or at least 97%) identical to amino acid sequence set forth as SEQ ID NO: 46.

In some embodiments, a protomer of a disclosed recombinant MuV F ectodomain trimer or recombinant MeV F ectodomain trimer, or a subunit of a MuV HN or MeV H multimer, can be linked to an encapsulin nanoparticle subunit to construct an encapsulin nanoparticle. The globular form of the encapsulin nanoparticle is made up of monomeric subunits; an example of the sequence of one such encapsulin subunit is provides as the amino acid sequence set forth as

(SEQ ID NO: 47)
MEFLKRSFAPLTEKQWQEIDNRAREIFKTQLYGRKFVDVEGPYGWEYAAH
PLGEVEVLSDENEVVKWGLRKSLPLIELRATFTLDLWELDNLERGKPNVD
LSSLEETVRKVAEFEDEVIFRGCEKSGVKGLLSFEERKIECGSTPKDLLE
AIVRALSIFSKDGIEGPYTLVINTDRWINFLKEEAGHYPLEKRVEECLRG
GKIITTPRIEDALVVSERGGDFKLILGQDLSIGYEDREKDAVRLFITETF
TFQVVNPEALILLKE.

In some embodiments, a protomer of a disclosed recombinant MuV F ectodomain trimer or recombinant MeV F ectodomain trimer, or a subunit of a MuV HN or MeV H multimer, can be linked to an encapsulin subunit including an amino acid sequence at least 80% (such as at least 85%, at least 90%, at least 95%, or at least 97%) identical to amino acid sequence set forth as SEQ ID NO: 47.

Encapsulin proteins are a conserved family of bacterial proteins also known as linocin-like proteins that form large protein assemblies that function as a minimal compartment to package enzymes. The encapsulin assembly is made up of monomeric subunits, which are polypeptides having a molecule weight of approximately 30 kDa. Following production, the monomeric subunits self-assemble into the globular encapsulin assembly including 60, or in some cases, 180 monomeric subunits. Methods of constructing encapsulin nanoparticles are further described (see, for example, Sutter et al., Nature Struct. and Mol. Biol., 15:939-947, 2008, which is incorporated by reference herein in its entirety). In specific examples, the encapsulin polypeptide is bacterial encapsulin, such as Thermotoga maritime or Pyrococcus furiosus or Rhodococcus erythropolis or Myxococcus xanthus encapsulin.

In some embodiments, a protomer of a disclosed recombinant MuV F ectodomain trimer or recombinant MeV F ectodomain trimer, or a subunit of a MuV HN or MeV H multimer, can be linked to a Sulfur Oxygenase Reductase (SOR) subunit to construct a recombinant SOR nanoparticle. In some embodiments, the SOR subunit can include the amino acid sequence set forth as

(SEQ ID NO: 48)
MEFLKRSFAPLTEKQWQEIDNRAREIFKTQLYGRKFVDVEGPYGWEYAAH
PLGEVEVLSDENEVVKWGLRKSLPLIELRATFTLDLWELDNLERGKPNVD
LSSLEETVRKVAEFEDEVIFRGCEKSGVKGLLSFEERKIECGSTPKDLLE
AIVRALSIFSKDGIEGPYTLVINTDRWINFLKEEAGHYPLEKRVEECLRG
GKIITTPRIEDALVVSERGGDFKLILGQDLSIGYEDREKDAVRLFITETF
TFQVVNPEALILLKE.

In some embodiments, a protomer of a disclosed recombinant MuV F ectodomain trimer or recombinant MeV F ectodomain trimer, or a subunit of a MuV HN or MeV H multimer, can be linked to a SOR subunit including an amino acid sequence at least 80% (such as at least 85%, at least 90%, at least 95%, or at least 97%) identical to amino acid sequence set forth as SEQ ID NO: 48.

SOR proteins are microbial proteins (for example from the thermoacidophilic archaeon Acidianus ambivalens that form 24 subunit protein assemblies. Methods of constructing SOR nanoparticlesare described in Urich et al., Science, 311:996-1000, 2006, which is incorporated by reference herein in its entirety. An example of an amino acid sequence of a SOR protein for use to make SOR nanoparticles is set forth in Urich et al., Science, 311:996-1000, 2006, which is incorporated by reference herein in its entirety.

For production purposes, the recombinant MuV F ectodomain or recombinant MeV F ectodomain, or the subunit of the MuV HN or MeV H multimer, linked to the nanoparticle subunit can include an N-terminal signal peptide that is cleaved during cellular processing. For example, the recombinant MuV F ectodomain protomer, or the recombinant MeV F ectodomain protomer, linked to the protein nanoparticle subunit can include a signal peptide at its N-terminus including, for example, a native MuV or MeV F signal peptide.

The protein nanoparticles can be expressed in appropriate cells (e.g., HEK 293 Freestyle cells) and fusion proteins are secreted from the cells self-assembled into nanoparticles. The nanoparticles can be purified using known techniques, for example by a few different chromatography procedures, e.g. Mono Q (anion exchange) followed by size exclusion (SUPEROSE® 6) chromatography.

The fusion proteins need not comprise the full-length sequence of a monomeric subunit polypeptide of a ferritin, encapsulin, SOR, or lumazine synthase protein. Portions, or regions, of the monomeric subunit polypeptide can be utilized so long as the portion comprises amino acid sequences that direct self-assembly of monomeric subunits into the globular form of the protein.

### II. Polynucleotides and Expression

Also provided are polynucleotides encoding any of the disclosed immunogens. For example, a polynucleotide encoding a protomer of a MuV F ectodomain trimer stabilized in the prefusion conformation, a protomer of a MeV F ectodomain trimer stabilized in the prefusion conformation, a chimera of one of these protomers linked to a MuV HN or MeV H ectodomain, or a subunit of a self-assembling protein nanoparticle containing a recombinant MuV or MeV F ectodomain. These polynucleotides include DNA, cDNA and RNA sequences, including vectors including the DNA, cDNA and RNA sequences, such as a DNA or RNA vector used for immunization. The genetic code can be used to construct a variety of functionally equivalent nucleic acids, such as nucleic acids which differ in sequence but which encode the same protein sequence, or encode a conjugate or fusion protein including the nucleic acid sequence.

An exemplary nucleic acid sequence encoding full-length MeV F protein is provided
as SEQ ID NO: 94:
atgggtctcaaggtgaacgtctctgccatattcatggcagtactgttaactctccaaacacccaccggtcaaatccattg
gggcaatctctctaagataggggtggtaggaataggaagtgcaagctacaaagttatgactcgttccagccatcaatcat
tagtcataaaattaatgcccaatataactctcctcaataactgcacgagggtagagattgcagaatacaggagactactg
agaacagttttggaaccaattagagatgcacttaatgcaatgacccagaatataagaccggttcagagtgtagcttcaag
taggagacacaagagatttgcgggagtagtcctggcaggtgcggccctaggcgttgccacagctgctcagataacagccg
gcattgcacttcaccagtccatgctgaactctcaagccatcgacaatctgagagcgagcctggaaactactaatcaggca
attgaggcaatcagacaagcagggcaggagatgatattggctgttcagggtgtccaagactacatcaataatgagctgat
accgtctatgaaccaactatcttgtgatttaatcggccagaagctcgggctcaaattgctcagatactatacagaaatcc
tgtcattatttggccccagcttacgggaccccatatctgcggagatatctatccaggctttgagctatgcgcttggagga
gacatcaataaggtgttagaaaagctcggatacagtggaggtgatttactgggcatcttagagagcagaggaataaaggc
ccggataactcacgtcgacacagagtcctacttaattgtcctcagtatagcctatccgacgctgtccgagattaaggggg
tgattgtccaccggctagagggggtctcgtacaacataggctctcaagagtggtataccactgtgcccaagtatgttgca
acccaagggtaccttatctcgaattttgatgagtcatcgtgtactttcatgccagaggggactgtgtgcagccaaaatgc
cttgtacccgatgagtcctctgctccaagaatgcctccgggggtccaccaagtcctgtgctcgtacactcgtatccgggt
cttttgggaaccggttcattttatcacaagggaacctaatagccaattgtgcatcaatcctttgcaagtgttacacaaca
ggaacgatcattaatcaagaccctgacaagatcctaacatacattgctgccgatcactgcccggtagtcgaggtgaacgg
cgtgaccatccaagtcgggagcaggaggtatccagacgctgtgtacttgcacagaattgacctcggtcctcccatattat
tggagaggttggacgtagggacaaatctggggaatgcaattgctaagttggaggatgccaaggaattgttggagtcatcg
gaccagatattgaggagtatgaaaggtttatcgagcacttgcatagtctacatcctgattgcagtgtgtcttggagggtt
gatagggatccccgctttaatatgttgctgcagggggcgttgtaacaaaaagggagaacaagttggtatgtcaagaccag
gcctaaagcctgatcttacgggaacatcaaaatcctatgtaaggtcgctctga
An exemplary nucleic acid sequence encoding full-length MuV F protein is provided
as SEQ ID NO: 95:
atgaaggccttttcagttacttgcttgggctttgcagtcttttcgtcttctatatgtgtgaatatcaacatcttgcagca
aattggatatatcaagcaacaagtcaggcaactaagctattactcacaaagttctagctcctacatagtggtcaagcttt
taccgaatatccaacccactgataacagctgtgaatttaagagtgtaactcaatacaataagaccttgagtaacttgctt
cttcccattgcagaaaacataaacaatattgcatcgccctcacctgggtcaagacgtcataaaaggtttgctggcattgc
cattggcattgctgcgctcggtgttgcgaccgcagcacaggtaactgccgctgtctcattagttcaagcacagacaaatg
cacgtgcaatagcggcgatgaaaaattcaatacaggcaactaatcgggcaatcttcgaagtgaaggaaggcacccaacag
ttagctatagcggtacaagcaatacaagaccacatcaatactattatgaacacccaattgaacaatatgtcttgtcagat
ccttgataaccagcttgcaacctacctaggattatacctaacagaattaacaacagtgtttcagccacaattaattaatc
cggcattgtcaccgattagtatacaagccttgaggtctttgcttggaagtatgacgcctgcagtggttcaagcaacatta
tctacgtcaatttctgctgctgaaatactaagtgccggtctaatggagggtcagattgtttctgttctgctagatgagat
gcagatgatagtcaagataaatattccaaccattgtcacacaatcaaatgcattggtgattgacttctactcaatttcga
gctttattaataatcaagaatccataattcaattgccagacaggatcctagagatcgggaatgaacaatggagctatcca
gctaaaaattgtaagttgacaagacaccacatattctgccaatacaatgaggcagagaggctgagcctagaatcaaaact
atgccttgcaggtaatataagtgcctgtgtgttctcacccatagcagggagttatatgaggcgatttgtagcactggatg
gaacaattgttgcaaactgtcgaagtctaacgtgtctatgcaagagtccatcttatcctatataccaacctgaccatcat
gcagtcacgaccattgatctaaccacatgtcaaacattgtccctagacggattggacttcagcattgtctctctaagcaa
catcacttacgctgagaaccttaccatttcattgtctcaaacaatcaatactcaacccattgacatatcaactgaactga
gtaaagttaatgcatccctccaaaatgccgttaagtacataaaggagagcaaccatcaactccaatctgtgagtgtaaat
tccaaaattggagctataattgtagcagccttagttttgagcatcctgtcaattatcatttcgctattgttttgctgctg
ggcttacattgcaactaaagaaatcagaagaatcaacttcaaaacaaatcatatcaacacaatatcgagtagtgtcgatg
atctcatcaggtactaa
An exemplary nucleic acid sequence encoding full-length MeV H protein is provided
as SEQ ID NO: 96:
atgtcaccacaacgagaccggataaatgccttctacaaagataacccccatcccaagggaagtaggatagtcattaacag
agaacatcttatgattgatagaccttatgttttgctggctgttctgtttgtcatgtttctgagcttgatcgggttgctag
ccattgcaggcattagacttcatcgggcagccatctacaccgcagagatccataaaagcctcagcaccaatctagatgta
actaactcaatcgagcatcaggtcaaggacgtgctgacaccactcttcaaaatcatcggtgatgaagtgggcctgaggac
acctcagagattcactgacctagtgaaattcatctctgacaagattaaattccttaatccggatagggagtacgacttca
gagatctcacttggtgtatcaacccgccagagagaatcaaattggattatgatcaatactgtgcagatgtggctgctgaa
gagctcatgaatgcattggtgaactcaactctactggagaccagaacaaccaatcagttcctagctgtctcaaagggaaa
ctgctcagggcccactacaatcagaggtcaattctcaaacatgtcgctgtccctgttagacttgtatttaggtcgaggtt
acaatgtgtcatctatagtcactatgacatcccagggaatgtatgggggaacttacctagtggaaaagcctaatctgagc
agcaaaaggtcagagttgtcacaactgagcatgtaccgagtgtttgaagtaggtgttatcagaaatccgggtttgggggc
tccggtgttccatatgacaaactatcttgagcaaccagtcagtaatgatctcagcaactgtatggtggctttgggggagc
tcaaactcgcagccctttgtcacggggaagattctatcacaattccctatcagggatcagggaaaggtgtcagcttccag
ctcgtcaagctaggtgtctggaaatccccaaccgacatgcaatcctgggtccccttatcaacggatgatccagtgataga
caggctttacctctcatctcacagaggtgttatcgctgacaatcaagcaaaatgggctgtcccgacaacacgaacagatg
acaagttgcgaatggagacatgcttccaacaggcgtgtaagggtaaaatccaagcactctgcgagaatcccgagtgggca
ccattgaaggataacaggattccttcatacggggtcttgtctgttgatctgagtctgacagttgagcttaaaatcaaaat
tgcttcgggattcgggccattgatcacacacggttcagggatggacctatacaaatccaaccacaacaatgtgtattggc
tgactatcccgccaatgaagaacctagccttaggtgtaatcaacacattggagtggataccgagattcaaggttagtccc
tacctcttcactgtcccaattaaggaagcaggcgaagactgccatgccccaacatacctacctgcggaggtggatggtga
tgtcaaactcagttccaatctggtgattctacctggtcaagatctccaatatgttttggcaacctacgatacttccaggg
ttgaacatgctgtggtttattacgtttacagcccaagccgctcattttcttacttttatccttttaggttgcctataaag
ggggtccccatcgaattacaagtggaatgcttcacatgggaccaaaaactctggtgccgtcacttctgtgtgcttgcgga
ctcagaatctggtggacatatcactcactctgggatggtgggcatgggagtcagctgcacagtcacccgggaagatggaa
ccaatcgcagatag
An exemplary nucleic acid sequence encoding full-length MuV HN protein is provided
as SEQ ID NO: 97:
atggagccctcgaaattcttcacaatatcggacagtgccacctttgcacctgggcctgtcagcaatgcggctgacaagaa
gacattccgaacctgcttccgaatactggtactatctgtacaagctgtcaccctcatattggttattgtcactttaggtg
agcttgtaaggatgatcaatgatcaaggcttgagcaatcagttgtcttcaattacagacaagataagagagtcagctact
atgattgcatctgctgtgggagtaatgaatcaagttattcatggagtaacggtatccttacccctacaaattgagggaaa
ccaaaatcaattgttagccacacttgccacaatctgcgccagccaaaaacaagtctcaaactgctctacaaacatcccct
tagtcaatgacctcaggtttataaatgggatcaataaatttattattgaagattacgcaactcatgatttctctatcggc
catccactcaatatgcccagctttatcccaactgcaacttcacccaatggttgcacaagaattccatccttttctttagg
taagacacactggtgctacacacataatgtaattaatgccaactgcaaggaccatacttcgtctaaccaatatgtgtcca
tggggattctcgttcagaccgcgtcagggtatcctatgttcaaaaccttaaaaatccaatatctcagtgatggcctgaat
cggaaaagctgctcaattgcaacagtccctgatgggtgcgcgatgtactgttatgtctctactcaacttgaaaccgacga
ctatgcggggtccagtccacccacccaaaaacttaccctgttattctataatgacaccgtcacagaaaggacaatatctc
catctggtcttgaagggaattgggctactttggtgccaggagtggggagtgggatatattttgagaataagttgatcttc
cctgcatatgggggtgtcttgcccaatagtacactcggggttaaatcagcaagagaattttttcggcctgttaatccata
taatccatgttcaggaccacaacaagatttagatcagcgtgctttgaggtcatacttcccaagttacttctctaatcgaa
gaatacagagtgcatttcttgtctgtgcctggaatcagatcctagttacaaattgtgagctagttgtcccctcaagcaat
cagacaatgatgggtgcagaagggagagttttattgatcaataatcgactattatattatcagagaagtaccagctggtg
gccgtatgaactcctctacgagatatcattcacatttacaaactctggtccatcatctgtaaatatgtcctggataccta
tatattcattcactcgtcctggttcaggcaattgcagtggtgaaaatgtgtgcccgactgcttgtgtgtcaggggtttat
cttgatccctggccattaactccatatagccaccaatcaggtattaacagaaatttctatttcacaggtgcactattaaa
ttcaagtacaactagagtaaatcctaccctttatgtctctgcccttaataatcttaaagtactagccccatatggtactc
aaggactgtttgcctcgtacaccacaaccacctgctttcaagataccggtgatgctagtgtgtattgtgtttatattatg
gagctagcatcaaatattgttggagaattccaaattctacctgtgctaactagattgactatcacttga

These exemplary nucleic acid sequences (or a corresponding RNA sequence) can be modified to encode any of the immunogens provided herein.

In several embodiments, the nucleic acid molecule encodes a precursor of a protomer of the MuV or MeV F ectodomain trimer or a promoter of the MeV F ectodomain trimer or a chimera of such a protomer with a MuV HN or MeV H ectodomain, or a subunit of a MuV HN or MeV H multimer, that, when expressed in an appropriate cell, is processed into a protomer of the F ectodomain trimer, or subunit of a MuV HN or MeV H multimer, that can self-assemble into the corresponding trimer or multimer. For example, the nucleic acid molecule can encode a protomer of the MuV or MeV F ectodomain trimer or a promoter of the MeV F ectodomain trimer including a N-terminal signal sequence for entry into the cellular secretory system that is proteolytically cleaved in the during processing of the recombinant F ectodomain in the cell.

In some embodiments, the nucleic acid molecule encodes a F₀ polypeptide that, when expressed in an appropriate cell, is processed into a protomer of the MuV or MeV F ectodomain trimer or a promoter of the MeV F ectodomain trimer including an F₂ polypeptide linked to a F₁ ectodomain, wherein the recombinant F₂-F₁ ectodomain protomer includes any of the prefusion-stabilizing modifications described herein, and optionally can be linked to a trimerization domain, such as a GCN4 trimerization domain and/or a T4 fibritin trimerization domain.

In some embodiments, the nucleic acid molecule encodes a full-length F₀ polypeptide that, when expressed in an appropriate cell, is processed into a protomer of the MuV or MeV F ectodomain trimer or a promoter of the MuV or MeV F ectodomain trimer including an F₂ polypeptide linked to a F₁ polypeptide including the F₁ transmembrane and cytosolic tail, wherein the recombinant F₂-F₁ ectodomain protomer includes any of the prefusion-stabilizing modifications described herein.

Exemplary nucleic acids can be prepared by cloning techniques. Examples of appropriate cloning and sequencing techniques, and instructions sufficient to direct persons of skill through many cloning exercises are known (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The polynucleotides encoding a protomer of the MuV or MeV F ectodomain trimer, or a subunit of a MuV HN or MeV H multimer, can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

Polynucleotide sequences encoding a protomer of the MuV or MeV F ectodomain trimer or a promoter of the MeV F ectodomain trimer or a chimera of such a protomer with a MuV HN or MeV H ectodomain, or a subunit of a MuV HN or MeV H multimer, can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

DNA sequences encoding the protomer of the MuV or MeV F ectodomain trimer or a promoter of the MeV F ectodomain trimer or a chimera of such a protomer with a MuV HN or MeV H ectodomain, or a subunit of a MuV HN or MeV H multimer, can be expressed in vitro by DNA transfer into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4^{th} Ed., Humana Press). Examples of commonly used mammalian host cell lines are VERO and HeLa cells, CHO cells, and WI38, BHK, and COS cell lines, although cell lines may be used, such as cells designed to provide higher expression, desirable glycosylation patterns, or other features. In some embodiments, the host cells include HEK293 cells or derivatives thereof, such as GnTI^{-/-} cells (ATCC® No. CRL-3022), or HEK-293F cells.

Transformation of a host cell with recombinant DNA can be carried out by conventional techniques. In some embodiments where the host is prokaryotic, such as, but not limited to, E. coli, competent cells which are capable of DNA uptake can be prepared from cells harvested after exponential growth phase and subsequently treated by the CaCl₂) method. Alternatively, MgCl₂ or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or viral vectors can be used. Eukaryotic cells can also be co-transformed with polynucleotide sequences encoding a disclosed antigen, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Viral Expression Vectors, Springer press, Muzyczka ed., 2011). Appropriate expression systems such as plasmids and vectors of use in producing proteins in cells including higher eukaryotic cells such as the COS, CHO, HeLa and myeloma cell lines.

In one non-limiting example, a disclosed immunogen is expressed using the pVRC8400 vector (described in Barouch et al., J. Virol., 79, 8828-8834, 2005, which is incorporated by reference herein).

Modifications can be made to a nucleic acid encoding a disclosed immunogen without diminishing its biological activity. Some modifications can be made to facilitate the cloning, expression, or incorporation of the targeting molecule into a fusion protein. Exemplary modifications include termination codons, a methionine added at the amino terminus to provide an initiation, site, additional amino acids placed on either terminus to create conveniently located restriction sites, or additional amino acids (such as poly His) to aid in purification steps.

In some embodiments, the nucleic acid encoding the protomer of the MuV or MeV F ectodomain trimer or the promoter of the MeV F ectodomain trimer or a chimera of such a protomer with a MuV HN or MeV H ectodomain, or a subunit of a MuV HN or MeV H multimer, can be expressed in cells under conditions where the protomers self-assemble into trimers which are secreted from the cells into the cell media, for example as described for RSV F proteins (see, e.g., PCT Pub. WO2014160463, McLellan et al., Science, 340:1113-1117, 2013; McLellan et al., Science, 342:592-598, 2013, each of which is incorporated by reference herein in its entirety). In such embodiments, the protomer contains a leader sequence (signal peptide) that causes the protein to enter the secretory system, and the signal peptide is cleaved and the protomers form a trimer, before being secreted in the cell media. The medium can be centrifuged and recombinant MuV or MeV F ectodomain trimer or recombinant MuV or MeV F ectodomain trimer or a chimera thereof with a MuV HN or MeV H ectodomain purified from the supernatant.

### III. Viral Vectors

A nucleic acid molecule encoding a disclosed immunogen can be included in a viral vector, for example, for expression of the immunogen in a host cell, or for immunization of a subject as disclosed herein. In some embodiments, the viral vectors are administered to a subject as part of a prime-boost vaccination. Typically such viral vectors include a nucleic acid molecule encoding an immunogen that contains a transmembrane domain. In several embodiments, the viral vectors are included in a vaccine, such as a primer vaccine or a booster vaccine for use in a prime-boost vaccination.

In some examples, the viral vector can be replication-competent. For example, the viral vector can have a mutation (e.g., insertion of nucleic acid encoding the protomer) in the viral genome that attenuates, but does not completely block viral replication in host cells.

In several embodiments, the viral vector can be delivered via the respiratory tract. For example, a hPIV vector, such as bovine parainfluenza virus (BPIV) vector (e.g., a BPIV1, BPIV2, or BPIV3 vector) or human hPIV vector (e.g., a hPIV3 vector), a metapneumovirus (MPV) vector, a Sendia virus vector, a New Castle Disease Virus (NCDV (vector), a mumps virus vector, a measles virus vector, or another paramyxovirus or pneumovirus vector is used to express a disclosed antigen.

Additional viral vectors are also available for expression of the disclosed antigens, including polyoma, i.e., SV40 (Madzak et al., 1992, J Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV and CMV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J Virol., 54:401-407), and human origin (Page et al., 1990, J Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif; Protein Sciences Corp., Meriden, Conn.; Stratagene, La Jolla, Calif).

### IV. Virus-Like Particles

In some embodiments, a virus-like particle (VLP) is provided that includes a disclosed immunogen. Typically such VLPs include an immunogen containing a transmembrane domain, for example, a recombinant MuV F ectodomain trimer with protomers containing a MuV F transmembrane domain and cytosolic tail, or a recombinant MeV F ectodomain trimer with protomers containing a MeV F transmembrane domain and cytosolic tail. VLPs lack the viral components that are required for virus replication and thus represent a highly attenuated, replication-incompetent form of a virus. However, the VLP can display a polypeptide (e.g., a recombinant MuV or MeV F ectodomain trimer) that is analogous to that expressed on infectious virus particles and can eliciting an immune response to MuV or MeV when administered to a subject. Exemplary virus like particles and methods of their production, as well as viral proteins from several viruses that are known to form VLPs, including human papillomavirus, HIV (Kang et al., Biol. Chem. 380: 353-64 (1999)), Semliki-Forest virus (Notka et al., Biol. Chem. 380: 341-52 (1999)), human polyomavirus (Goldmann et al., J. Virol. 73: 4465-9 (1999)), rotavirus (Jiang et al., Vaccine 17: 1005-13 (1999)), parvovirus (Casal, Biotechnology and Applied Biochemistry, Vol 29, Part 2, pp 141-150 (1999)), canine parvovirus (Hurtado et al., J. Virol. 70: 5422-9 (1996)), hepatitis E virus (Li et al., J. Virol. 71: 7207-13 (1997)), and Newcastle disease virus. The formation of such VLPs can be detected by any suitable technique. Examples of suitable techniques for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation.

### V. Immunogenic Compositions

Immunogenic compositions comprising a disclosed immunogen (e.g., recombinant MuV F ectodomain trimer, a recombinant MeV F ectodomain trimer, or corresponding fusions with MuV HN ectodomain or MeV H ectodomain, or a MuV HN or MeV H multimer) and a pharmaceutically acceptable carrier are also provided. Such compositions can be administered to subjects by a variety of administration modes, for example, intramuscular, subcutaneous, intravenous, intra-arterial, intra-articular, intraperitoneal, or parenteral routes. In several embodiments, a pharmaceutical composition including one or more of the disclosed immunogens are immunogenic compositions. Actual methods for preparing administrable compositions are described in more detail in such publications as Remingtons Pharmaceutical Sciences, 19^{th} Ed., Mack Publishing Company, Easton, Pennsylvania, 1995.

Thus, an immunogen described herein can be formulated with pharmaceutically acceptable carriers to help retain biological activity while also promoting increased stability during storage within an acceptable temperature range. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The immunogenic composition may optionally include an adjuvant to enhance an immune response of the host. Adjuvants, such as aluminum hydroxide (e.g., ALHYDROGEL®, available from Brenntag Biosector, Copenhagen, Denmark and Amphogel®, Wyeth Laboratories, Madison, NJ), Freund's adjuvant, MPL™ (3-O-deacylated monophosphoryl lipid A; Corixa, Hamilton, IN) and IL-12 (Genetics Institute, Cambridge, MA), TLR agonists (such as TLR-9 agonists, for example cytidine-phospho-guanosine oligodeoxynucleotide (CpG-ODN)1018), among many other suitable adjuvants well known in the art, can be included in the compositions. Suitable adjuvants are, for example, toll-like receptor agonists, alum, AlPO4, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product.

In some instances, the adjuvant formulation is a mineral salt, such as a calcium or aluminum (alum) salt, for example calcium phosphate, aluminum phosphate or aluminum hydroxide. In some embodiments, the disclosed immunogen comprises one or more phosphoserine modifications and is used with a Alum adjuvant. In some embodiments, the adjuvant includes an oil and water emulsion, e.g., an oil-in-water emulsion (such as MF59 (Novartis) or AS03 (GlaxoSmithKline). One example of an oil-in-water emulsion comprises a metabolisable oil, such as squalene, a tocol such as a tocopherol, e.g., alpha-tocopherol, and a surfactant, such as sorbitan trioleate (Span 85) or polyoxyethylene sorbitan monooleate (Tween 80), in an aqueous carrier.

In some instances, it may be desirable to combine a disclosed immunogen with other pharmaceutical products (e.g., vaccines) which induce protective responses to other agents. For example, a composition including a recombinant MuV F ectodomain trimer, a recombinant MeV F ectodomain trimer, or corresponding fusions with MuV HN ectodomain or MeV H ectodomain as described herein can be can be administered simultaneously (typically separately) or sequentially with other vaccines recommended by the Advisory Committee on Immunization Practices (ACIP; cdc.gov/vaccines/acip/index.html) for the targeted age group (e.g., infants from approximately one to six months of age). As such, a disclosed immunogen described herein may be administered simultaneously or sequentially with vaccines against, for example, hepatitis B (HepB), diphtheria, tetanus and pertussis (DTaP), pneumococcal bacteria (PCV), Haemophilus influenzae type b (Hib), polio, influenza and rotavirus.

In some embodiments, the composition can be provided as a sterile composition. The immunogenic composition typically contains an effective amount of a disclosed immunogen and can be prepared by conventional techniques. Typically, the amount of immunogen in each dose of the immunogenic composition is selected as an amount which induces an immune response without significant, adverse side effects. In some embodiments, the composition can be provided in unit dosage form for use to induce an immune response in a subject, for example, to inhibit MuV and/or MeV infection in the subject. A unit dosage form contains a suitable single preselected dosage for administration to a subject, or suitable marked or measured multiples of two or more preselected unit dosages, and/or a metering mechanism for administering the unit dose or multiples thereof.

### VI. Methods of Inducing an Immune Response

The disclosed immunogens (e.g., recombinant MuV F ectodomain trimer, a recombinant MeV F ectodomain trimer, or corresponding fusions with MuV HN ectodomain or MeV H ectodomain, MuV HN or MeV H multimer, a nucleic acid molecule (such as an RNA molecule) encoding a disclosed immunogen, or a protein nanoparticle or virus like particle comprising the immunogen) can be administered to a subject to induce an immune response to MuV and/or MeV in the subject. In a particular example, the subject is a human. The immune response can be a protective immune response, for example a response that inhibits subsequent infection with MuV and/or MeV. Elicitation of the immune response can also be used to treat or inhibit MuV and/or MeV infection and illnesses associated therewith.

A subject can be selected for treatment that has, or is at risk for developing MuV or MeV infection, for example because of exposure or the possibility of exposure to MuV or MeV. Following administration of a disclosed immunogen, the subject can be monitored for the MuV and/or MeV infection or symptoms associated therewith, or both.

Typical subjects intended for treatment with the therapeutics and methods of the present disclosure include humans. In some embodiments, the subject is a human subject that is seronegative for MuV and/or MeV specific antibodies. In some embodiments, the subject is a human subject that is seropositive for MuV and/or MeV specific antibodies, and the immunogen is administered to boost the immune response to MeV and/or MuV in the subject. To identify subjects for treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize MuV and/or MeV infection. These and other routine methods allow the clinician to select patients in need of therapy using the methods and immunogenic compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

The administration of a disclosed immunogen can be for prophylactic or therapeutic purpose. When provided prophylactically, the immunogen can be provided in advance of any symptom, for example in advance of infection. The prophylactic administration serves to prevent or ameliorate any subsequent infection. In some embodiments, the methods can involve selecting a subject at risk for contracting MuV and/or MeV infection, and administering a therapeutically effective amount of a disclosed immunogen to the subject. The immunogen can be provided prior to the anticipated exposure to MuV and/or MeV so as to attenuate the anticipated severity, duration or extent of an infection and/or associated disease symptoms, after exposure or suspected exposure to the virus, or after the actual initiation of an infection. Populations that benefit from prophylactic use of the disclosed immunogen (for example, as a boost immunization) include children entering school (e.g., 5 years old) and adolescents entering high school or college or military (e.g., 15-18 years old). Transplant recipients may also need to be revaccinated or immunocompromised children like HIV+ would benefit from a protein vaccine as opposed to a live-attenuated virus that may not be safe including pregnant women.

When provided therapeutically, the disclosed immunogens are provided at or after the onset of a symptom of MuV and/or MeV infection, or after diagnosis of MuV and/or MeV infection. Treatment of MuV by inhibiting MuV replication or infection can include delaying and/or reducing signs or symptoms of MuV infection in a subject. Treatment of MeV by inhibiting MeV replication or infection can include delaying and/or reducing signs or symptoms of MeV infection in a subject. In some examples, treatment using the methods disclosed herein prolongs the time of survival of the subject.

In some embodiments, administration of a disclosed immunogen to a subject can elicit the production of an immune response that is protective against or reduces symptoms of disease when the subject is subsequently infected or re-infected with a wild-type MuV and/or MeV. While the naturally circulating virus may still be capable of causing infection there can be a reduced possibility of serious or life-threatening symptoms as a result of the vaccination and a possible boosting of resistance by subsequent infection by wild-type virus. Following vaccination, there are detectable levels of host engendered serum and secretory antibodies which are capable of neutralizing homologous (of the same subgroup) wild-type virus in vitro and in vivo. In many instances the host antibodies will also neutralize wild-type virus of a different, non-vaccine subgroup.

The immunogens described herein, and immunogenic compositions thereof, are provided to a subject in an amount effective to induce or enhance an immune response against MuV and/or MeV in the subject, preferably a human. The actual dosage of disclosed immunogen will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

An immunogenic composition including one or more of the disclosed immunogens can be used in coordinate (or prime-boost) vaccination protocols or combinatorial formulations. In certain embodiments, novel combinatorial immunogenic compositions and coordinate immunization protocols employ separate immunogens or formulations, each directed toward eliciting an anti-viral immune response, such as an immune response to MuV F protein and/or MeV F protein. Separate immunogenic compositions that elicit the anti-viral immune response can be combined in a polyvalent immunogenic composition administered to a subject in a single immunization step, or they can be administered separately (in monovalent immunogenic compositions) in a coordinate (or prime-boost) immunization protocol.

There can be several boosts, and each boost can be a different disclosed immunogen. In some examples that the boost may be the same immunogen as another boost, or the prime. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example a relatively large dose in a primary immunization and then a boost with relatively smaller doses.

In some embodiments, the boost can be administered about two, about three to eight, or about four, weeks following the prime, or about several months after the prime. In some embodiments, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., inhibition of MuV and/or MeV infection or improvement in disease state (e.g., reduction in viral load). If such monitoring indicates that vaccination is sub-optimal, the subject can be boosted with an additional dose of immunogenic composition, and the vaccination parameters can be modified in a fashion expected to potentiate the immune response.

In some embodiments, the prime-boost method can include DNA-primer and protein-boost vaccination protocol to a subject. The method can include two or more administrations of the nucleic acid molecule or the protein.

For protein therapeutics, typically, each human dose will comprise 1-1000 pg of protein, such as from about 1 μg to about 100 μg, for example, from about 1 μg to about 50 μg, such as about 1 μg, about 2 μg, about 5 μg, about 10 μg, about 15 μg, about 20 μg, about 25 μg, about 30 μg, about 40 μg, or about 50 μg.

The amount utilized in an immunogenic composition is selected based on the subject population (e.g., infant or elderly). An optimal amount for a particular composition can be ascertained by standard studies involving observation of antibody titers and other responses in subjects. It is understood that an effective amount of a disclosed immunogen, such as a recombinant MuV or MeV F ectodomain trimer or recombinant MeV F ectodomain trimer or a chimera thereof with a MuV HN or MeV H ectodomain, viral vector, or nucleic acid molecule in a immunogenic composition, can include an amount that is ineffective at eliciting an immune response by administration of a single dose, but that is effective upon administration of multiple dosages, for example in a prime-boost administration protocol.

Upon administration of a disclosed immunogen the immune system of the subject typically responds to the immunogenic composition by producing antibodies specific for viral protein. Such a response signifies that an immunologically effective dose was delivered to the subject.

For each particular subject, specific dosage regimens can be evaluated and adjusted over time according to the individual need and professional judgment of the person administering or supervising the administration of the immunogenic composition. The dosage and number of doses will depend on the setting, for example, in an adult or anyone primed by prior MuV and/or MeV infection or immunization, a single dose may be a sufficient booster. In naïve subjects, in some examples, at least two doses would be given, for example, at least three doses. In some embodiments, an annual boost is given, for example, along with an annual influenza vaccination.

In some embodiments, the antibody response of a subject will be determined in the context of evaluating effective dosages/immunization protocols. In most instances it will be sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum which bind to an antigen including, for example, a MuV F protein and/or a MeV F protein.

Determination of effective dosages is typically based on animal model studies followed up by human clinical trials and is guided by administration protocols that significantly reduce the occurrence or severity of targeted disease symptoms or conditions in the subject, or that induce a desired response in the subject (such as a neutralizing immune response). Suitable models in this regard include, for example, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer an effective amount of the composition (for example, amounts that are effective to elicit a desired immune response or alleviate one or more symptoms of a targeted disease). In alternative embodiments, an effective amount or effective dose of the composition may simply inhibit or enhance one or more selected biological activities correlated with a disease or condition, as set forth herein, for either therapeutic or diagnostic purposes.

Administration of an immunogenic composition that elicits an immune response to reduce or prevent an infection, can, but does not necessarily completely, eliminate such an infection, so long as the infection is measurably diminished. For example, administration of an effective amount of the agent can decrease the MuV or MeV infection (for example, as measured by infection of cells, or by number or percentage of subjects infected by MuV or MeV by a desired amount, for example by at least 10%, at least 20%, at least 50%, at least 60%, at least 70%, at least 80%, at least 90%, at least 95%, at least 98%, or even at least 100% (elimination or prevention of detectable MuV or MeV infection, as compared to a suitable control, also referred to as sterilizing immunity).

In some embodiments, administration of an effective amount of one or more of the disclosed immunogens to a subject induces a neutralizing immune response in the subject. To assess neutralization activity, following immunization of a subject, serum can be collected from the subject at appropriate time points, frozen, and stored for neutralization testing. Methods to assay for neutralization activity include, but are not limited to, plaque reduction neutralization titer (PRNT) assays, microneutralization assays, flow cytometry-based assays, single-cycle infection assays. In some embodiments, the serum neutralization activity can be assayed using a panel of MuV and/or MeV pseudoviruses.

One approach to administration of nucleic acids is direct immunization with plasmid DNA, such as with a mammalian expression plasmid. Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 g encapsulated in ISCOMS™ have been found to produce Class I mediated CTL responses (Takahashi et al., Nature 344:873, 1990).

In some embodiments, a plasmid DNA vaccine is used to express a disclosed immunogen in a subject. For example, a nucleic acid molecule encoding a disclosed immunogen can be administered to a subject to elicit an immune response to the F protein of MuV or MeV. In some embodiments, the nucleic acid molecule can be included on a plasmid vector for DNA immunization, such as the pVRC8400 vector (described in Barouch et al., J. Virol, 79, 8828-8834, 2005, which is incorporated by reference herein).

In another approach to using nucleic acids for immunization, a disclosed immunogen can be expressed by attenuated viral hosts (such as an attenuated MuV or MeV vector) or vectors or bacterial vectors. Recombinant vaccinia virus, adeno-associated virus (AAV), herpesvirus, retrovirus, cytogmeglovirus, paramyxovirus, pneumovirus, or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In another example, a disclosed immunogen can be administered to a subject using RNA immunization, such as with a lipid-encapsulated mRNA immunization platform (see, e.g., Roth et al., “A Modified mRNA Vaccine Targeting Immunodominant NS Epitopes Protects Against Dengue Virus Infection in HLA Class I Transgenic Mice,” Front Immunol., Jun. 21, 2019, Vol. 10, Article 1424; Jagger et al., J Infect Dis, “Protective Efficacy of Nucleic Acid Vaccines Against Transmission of Zika Virus During Pregnancy in Mice,” jiz338, Jul. 1, 2019; Feldman et al., “mRNA vaccines against H10N8 and H7N9 influenza viruses of pandemic potential are immunogenic and well tolerated in healthy adults in phase 1 randomized clinical trials,” Vaccine, 37(25), 3326-3334, 2019; and Hasset et al., “Optimization of Lipid Nanoparticles for Intramuscular Administration of mRNA Vaccines,” Mol Ther Nucleic Acids, 15: 1-11, 2019.

In one embodiment, a nucleic acid encoding a protomer of a disclosed MuV F or MeV F ectodomain trimer is introduced directly into cells. For example, the nucleic acid can be loaded onto gold microspheres by standard methods and introduced into the skin by a device such as Bio-Rad's HELIOS^{T}M Gene Gun. The nucleic acids can be “naked,” consisting of plasmids under control of a strong promoter. Typically, the DNA is injected into muscle, although it can also be injected directly into other sites. Dosages for injection are usually around 0.5 g/kg to about 50 mg/kg, and typically are about 0.005 mg/kg to about 5 mg/kg (see, e.g., U.S. Pat. No. 5,589,466).

In another embodiment, an mRNA-based immunization protocol can be used to deliver a nucleic acid encoding a disclosed immunogen directly into cells. In some embodiments, nucleic acid-based vaccines based on mRNA may provide a potent alternative to the previously mentioned approaches. mRNA vaccines preclude safety concerns about DNA integration into the host genome and can be directly translated in the host cell cytoplasm. Moreover, the simple cell-free, in vitro synthesis of RNA avoids the manufacturing complications associated with viral vectors. Two exemplary forms of RNA-based vaccination that can be used to deliver a nucleic acid encoding a disclosed immunogen include conventional non-amplifying mRNA immunization (see, e.g., Petsch et al., “Protective efficacy of in vitro synthesized, specific mRNA vaccines against influenza A virus infection,” Nature biotechnology, 30(12):1210-6, 2012) and self-amplifying mRNA immunization (see, e.g., Geall et al., “Nonviral delivery of self-amplifying RNA vaccines,” PNAS, 109(36): 14604-14609, 2012; Magini et al., “Self-Amplifying mRNA Vaccines Expressing Multiple Conserved Influenza Antigens Confer Protection against Homologous and Heterosubtypic Viral Challenge,” PLoS One, 11(8):e0161193, 2016; and Brito et al., “Self-amplifying mRNA vaccines,” Adv Genet., 89:179-233, 2015).

In some embodiments, a lipid nanoparticle including mRNA encoding a disclosed immunogen is used in the method of eliciting an immune response, for example, as described in WO2017070626, US2019/0192646, and for the mRNA-1273 vaccine described in Jackson et al. “An mRNA vaccine against SARS-CoV2-preliminary report,” N. Engl. J. Med., 383(20):1920-1931, 2020, each of which is incorporated by reference herein. As described in WO2017070626, the mRNA encoding the immunogen can be formulated in lipid nanoparticles with 50 mol % ionizable lipid, 10 mol % DSPC, 38.5 mol % cholesterol, and 1.5 mol % (PEG2000 DMG). Further, the mRNA encoding the immunogen can be a modified mRNA with 1-methylpseudouridine in place of uridine and a 7mG(5′)ppp(5′)N1mpNp cap (enzymatic), as well as a 5′UTR, a 3′UTR, and a polyA tail.

### EXAMPLES

The following examples are provided to illustrate particular features of certain embodiments, but the scope of the claims should not be limited to those features exemplified.

### Example 1

### MuV F Proteins Stabilized in a Prefusion Conformation and Fusions Thereof with MuV HN Ectodomain

This example illustrates embodiments of a MuV F ectodomain trimer stabilized in a prefusion conformation by one or more amino acid substitutions. Further provided are MuV F ectodomain trimers linked to a MuV HN ectodomain. The prefusion-stabilized MuV F ectodomain trimers and corresponding fusions with MuV HN ectodomain are useful, for example, for inducing a neutralizing immune response to MuV in a subject.

Before the introduction of mumps-containing vaccines in the late 1960s, mumps caused widespread morbidity characterized by fever, parotitis and less commonly, orchiditis, meningitis, encephalitis and deafness.

The combination measles, mumps and rubella vaccine (MMR) dramatically reduced the incidence of mumps throughout the world. Two doses of MMR vaccine are approximately 88% effective for the prevention of mumps disease however since 2006, there has been a resurgence in the number of cases of mumps disease in the world among highly-vaccinated populations, with >30,000 affected individuals in the U.S. Contributing factors may include waning immunity, poorly effective antibody responses, and antigenic differences between the Jeryl Lynn strain used in the MMR vaccine and circulating wildtype strains. The predominant mumps genotype characterizing outbreaks in the USA and Europe in recent years has been genotype G.

As described herein, structure-based design was used to engineer a MuV F glycoprotein stabilized in the prefusion conformation. A crystal structure of mumps fusion glycoprotein at 2.16 Å resolution reveals the basis for prefusion conformational stabilization. Potent cross-mumps genotype plaque reduction neutralizing titers (PRNT) were elicited in mice from the mumps prefusion-stabilized F glycoprotein or a chimeric fusion glycoprotein of prefusion-stabilized mumps F trimer linked to genotype G mumps hemagglutinin neuraminidase (HN). The prefusion F-HN mumps chimera could elicit the highest PRNT to genotypes A, G and H mumps viruses, greater than 100-fold the reported human protective titer. Additionally, monoclonal antibodies to mumps prefusion F and HN were isolated from immunized mice, which were capable of neutralizing genotype G mumps virus with a spectrum of potencies. Structural and binding analyses of these prefusion F-specific antibodies revealed binding to four discrete neutralizing antigenic sites. The engineered immunogens are vaccine candidates for mumps either as novel or as booster vaccines.

### Results

Disulfide bond and membrane-proximal coiled coil stabilization robustly stabilize and allow production of a soluble prefusion mumps F trimer.

When produced in cells, the MuV F ectodomain linked to a C-terminal GCN4 trimerization domain forms trimers that spontaneously transition to the prefusion conformation. Unstabilized recombinant MuV F-GCN4 is so unstable, 100% of molecules have transitioned to the postfusion conformation at the point of evaluation (EM). Also protein expression is substantially reduced without stabilization. Accordingly, structure-based vaccine design was used to identify mutations for the stabilization of the MuV F ectodomain in a prefusion conformation and also to eliminate the F₁/F₂ cleavage site to produce a “single chain” MuV F protein with increased expression.

The crystal structures of the simian prefusion parainfluenza virus 5 (PIV5) F glycoprotein (PDB IDs 4GIP, 4WSG) (Welch, B. D. et al. Proc Natl Acad Sci USA 109, 16672-16677, 2012) were used to construct a homology model for the prefusion mumps F protein, consisting of three intertwined monomers forming a quaternary assembly of DI, DII, DIII and HRB domains.

Multiple stabilization strategies were employed to “lock” the MuV F ectodomain in the prefusion conformation, including introduction of disulfide bonds and proline substitutions. The selection of residues to mutate to cysteines in MuV F were based on homology design from the PIVS prefusion F structure (PDB 4WSG), based on residues that would be predicted to undergo conformational change in transitioning from prefusion to postfusion conformation. The residue pairs Cbeta atoms were identified to be within 5 angstrom and orientated such that the formation of a disulfide bond might be possible. In total, approximately 60 different mutants were designed, expressed, purified, assessed for expression level, and assessed for prefusion conformation by negative stain EM.

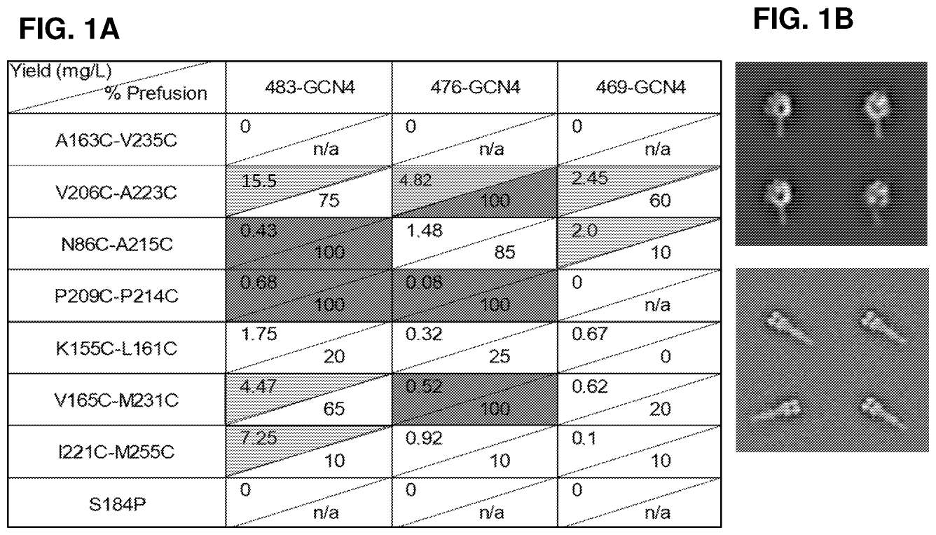

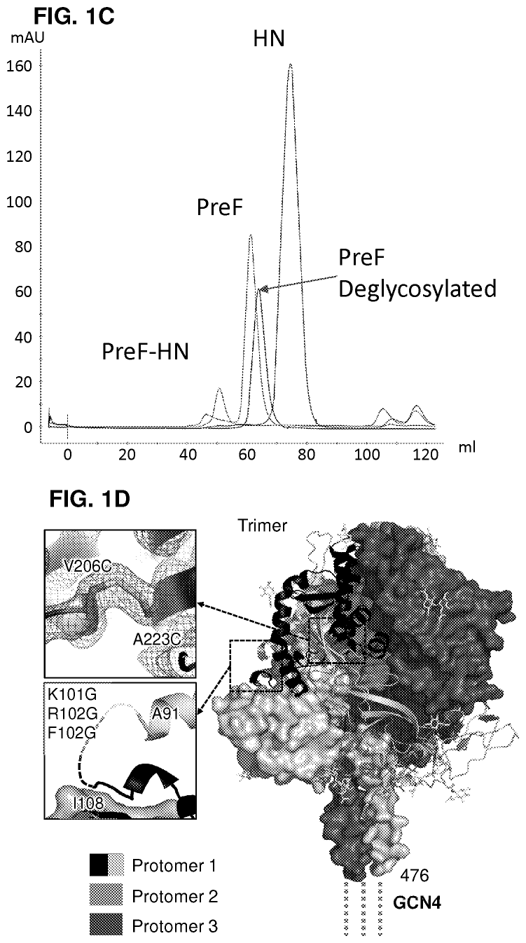

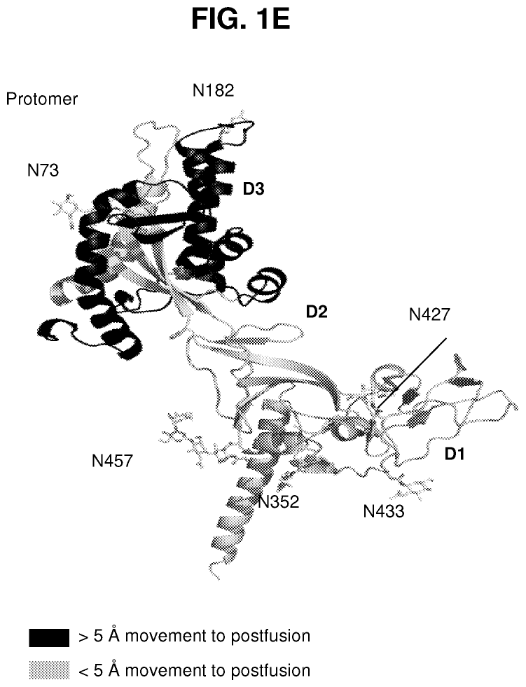

The mutations were introduced into a MuV F ectodomain (based on C-terminal truncations at MuV F position 469, 476, or 483, and linked to a C-terminal GCN4 trimerization domain, and the resulting mutants were screened as noted above. The ectodomain also included a mutation to remove the F₁/F₂ furin cleavage site. The prefusion stabilizing mutations assessed included: cysteine substitutions at one or more of MuV F positions 86 and 215, 155 and 161, 163 and 235, 165 and 231, 206 and 223, 209 and 214, 221 and 255 that form a non-natural disulfide bond; and a proline substitution at MuV F position 184. Relevant sequences are shown below. In FIG. 1A, a summary of the initial successful prefusion-stabilizing mutations are reported.

The expression and purification of the single chain and prefusion-stabilized MuV F proteins showed a substantial increase in expression level compared to the unmodified MuV F.

As illustrated in FIGS. 1A and B, negative stain EM can be used to distinguish MuV F ectodomain trimers that are in the prefusion conformation from those that are in the postfusion conformation.

By creating a matrix of disulfide substitutions and C-terminal coiled-coil-GCN4 attachment positions, the level of protein expression and the proportion of protein adopting the prefusion or postfusion trimer conformation was evaluated using negative-stain EM (FIG. 1A). It was observed that while five combinations of disulfide bonds and GCN4 attachment positions gave rise to 100% prefusion trimers, one such combination gave rise to high-yield protein expression (V206C-A223C with 476-GCN4) at about 4.8 mg/L from Expi293 cells. The negative stain EM 2D average of this design (FIG. 1B, top inset) contrasts with the postfusion mumps F glycoprotein trimer (FIG. 1B, lower inset) and was consistent with prior observations for parainfluenza virus F protein conformations.

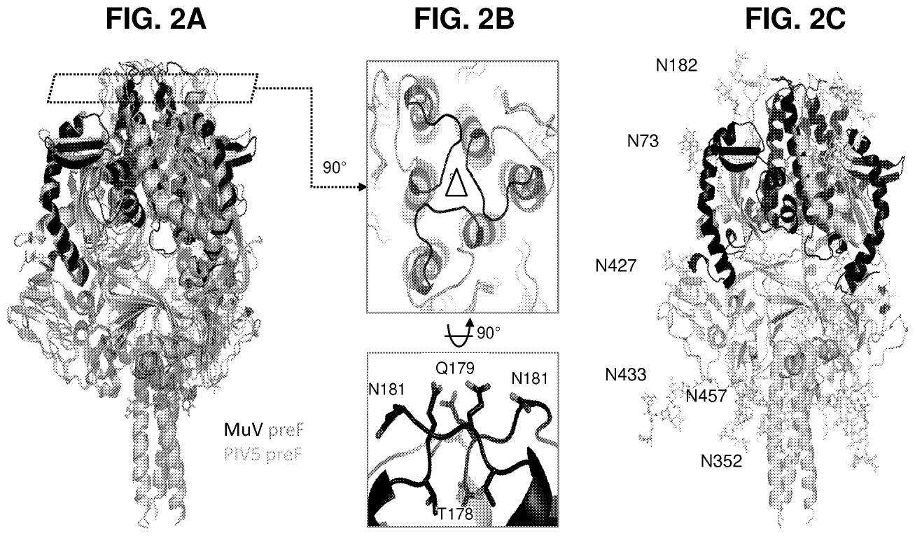

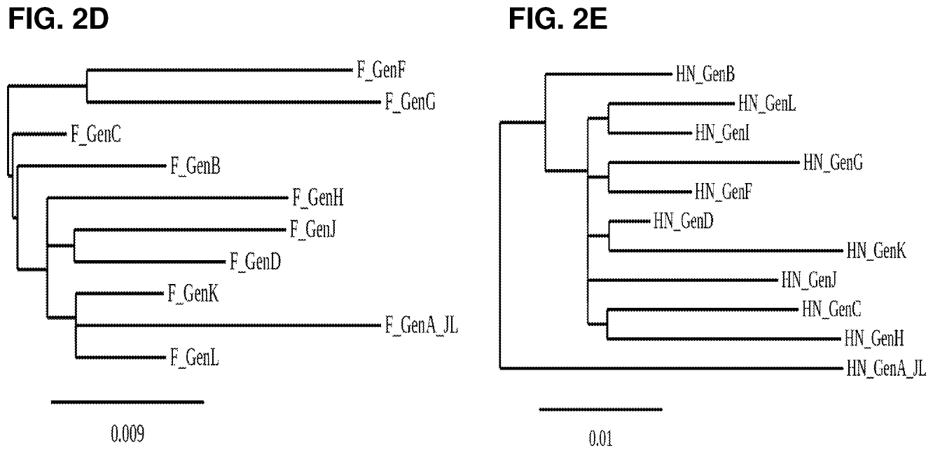

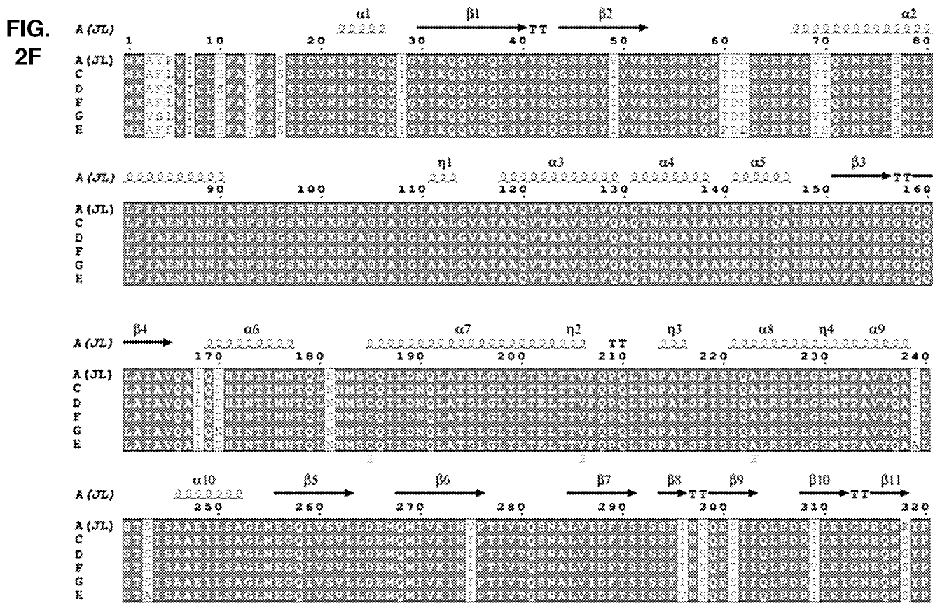

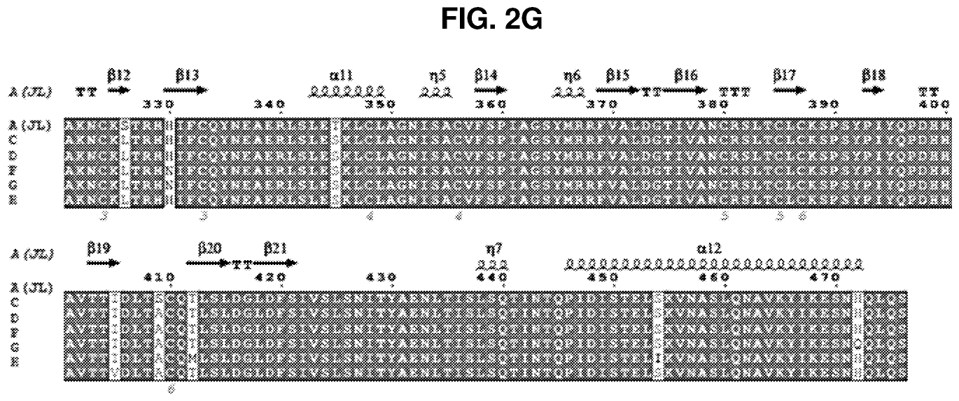

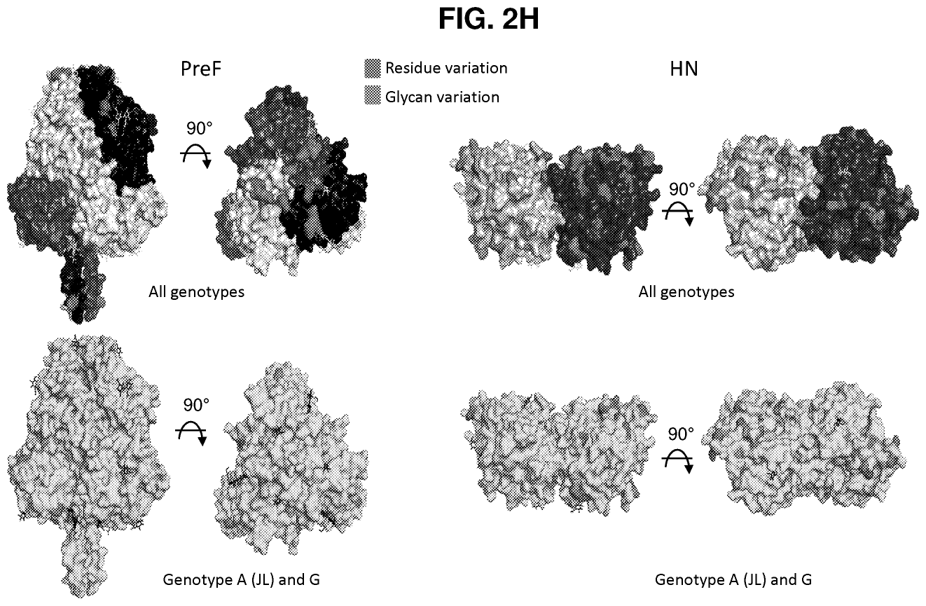

Crystal structure of prefusion mumps F glycoprotein trimer to 2.16 Å resolution reveals a stabilizing disulfide design and the location of polymorphic residues. Size-exclusion chromatography of the prefusion-stabilized mumps F protein trimer (MuV F V206C-A223C-GGG-476-GCN4, SEQ ID NO: 11) revealed a homogeneous peak (FIG. 1C), which when expressed in the presence of kifunensine, could be deglycosylated and crystallized. The 3-dimensional coordinates for the structure are provided herewith in Table 1. The x-ray structure to 2.16 Å resolution (FIGS. 1D and 1E) revealed a similar overall architecture to prefusion PIVS and other paramyxovirus prefusion F trimers (Stewart-Jones et al., Proc Natl Acad Sci USA 115, 12265-12270, 2018; Welch, B. D. et al. Proc Natl Acad Sci USA 109, 16672-16677, 2012; Xu K. et al. PLoS Pathog 11(12):e1005322, 2015), however adopts a ‘closed cap’ structure comprising of loops N177-S184 at the trimer apex of the mumps prefusion F trimer, formed by interactions between side chains T178, Q179 and N181 (FIGS. 2A and 2B). The remaining N-acetyl glucosamine moieties from the six glycans at positions N73, N182, N352, N427, N433 and N457 were visible in the electron density and a glycosylated model built (FIG. 2C). The V206C-A223C disulfide was clearly defined by the electron density with a Cα-Cα atomic distance of 4.8 Å whereas in the homologous postfusion PIV3 F trimer structure [PDB ID 1ZTM], Cα-Cα atoms are positioned 5.8 Å apart. The DI-DIII domains enclosed a large aqueous cavity measuring ~20,000 Å³ in volume and protomers bury a 6,500 Å² interface, similar to that observed in PIVS, PIV3 and nipah prefusion F structures (Stewart-Jones et al., Proc Nat Acad Sci USA 115, 12265-12270, 2018; Welch, B. D. et al. Proc Nat Acad Sci USA 109, 16672-16677, 2012; Xu K. et al. PLoSPathog 11(12):e1005322, 2015). The residues thought to undergo conformational change between prefusion and postfusion conformations (based on PIV5 pre-F structure [PDB ID 4WSG] and PIV3 post-F [PDB ID 1ZTM]) are shown in black in FIG. 1E and correspond to MuV F residues 92-253.

While the sequence identities of mumps F and HN are relatively high between genotypes (FIGS. 2A-2C), polymorphic variation to the structures of prefusion mumps F and HN was mapped (FIG. 2D). Mapping the variation to prefusion F for all genotypes and specifically between genotype G and Jeryl Lynn (genotype A), much of the protein surface exposed for antibody recognition was conserved, while numerous variable amino acids were located within the aqueous cavity in the core of the prefusion trimer (FIG. 2D, left). In contrast, the mumps HN dimer structures revealed that most polymorphic amino acids are exposed to the solvent (FIG. 2D, right), including a glycan variation at position N464 between Jeryl Lynn and genotype G HNs. The predominance of solvent-exposed polymorphic residues on HN versus prefusion F suggests that genotype G resistance to Jeryl Lynn vaccine-elicited humoral immunity could be accounted for more from HN than from prefusion F.

Cross-strain effectiveness of stabilizing mutations. To show that the prefusion stabilizing mutations were effective for F from across MuV strains, these mutations were tested in F from several different MuV strains. MuV F V206C-A223C-GGG-476-GCN4 (SEQ ID NO: 11) is based on a genotype C MuV F. Introduction of these prefusion-stabilizing mutations into a genotype A (Jeryl Lynn) MuV F protein (MuV-JL F 206C-A223C-GGG-476-GCN4 (SEQ ID NO: 26) and genotype G MuV F protein (MuV-IL17 F 206C-A223C-GGG-476-GCN4 (SEQ ID NO: 51) also provided prefusion stabilization. Further, introduction of these prefusion-stabilizing mutations into F protein from the following MuV stains also resulted in prefusion stabilization as measured by negative stain EM and/or prefusion specific antibody binding: Canada (URABE), Albany (genotype A), Hoshino (genotype B), India (genotype C), Netherlands (genotype D), China (genotype F), NethL11 (genotype G), NY 14 (genotype G), IA14 (genotype G), MA16 (genotype G), LA17 (genotype G), IL17 (genotype G), Virginia (genotype H), Taiwan (genotype J), Taiwan (genotype K), Netherlands (genotype L), MG15 (genotype A)

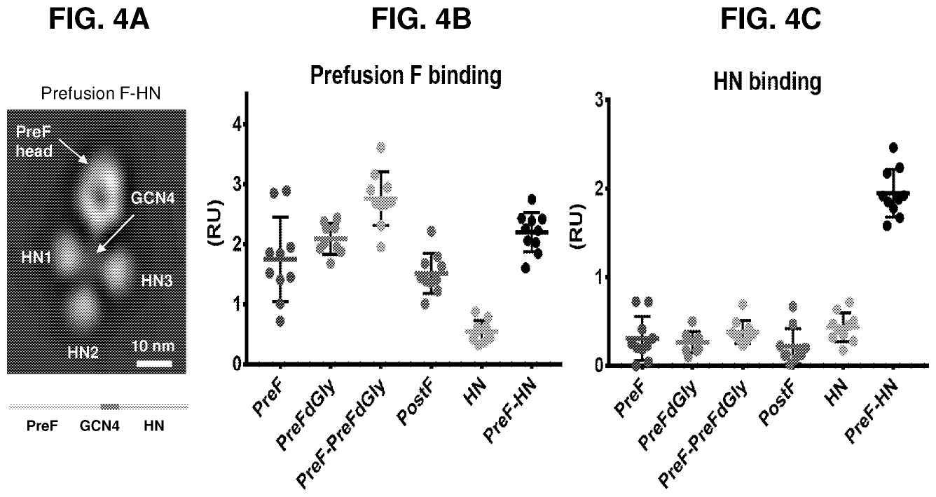

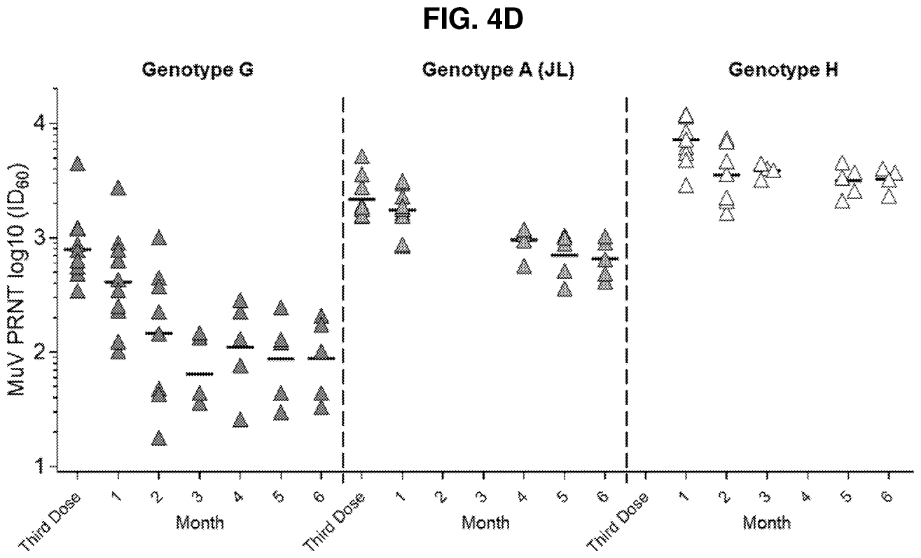

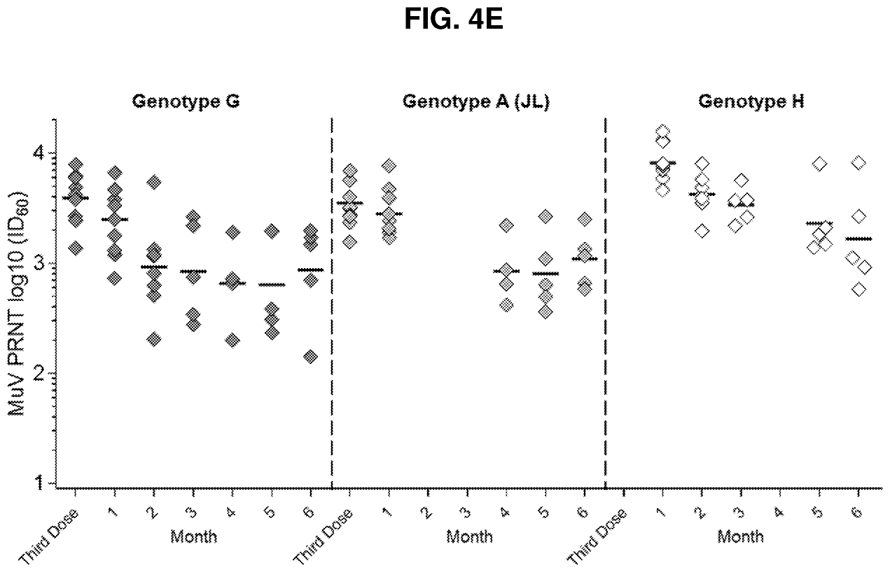

F-HN chimera. To increase the immunogenic footprint of the MuV F ectodomain trimer, a MuV HN ectodomain was genetically fused to the C-terminus of the trimerization domain of each protomer of the trimer. The format is illustrated in FIG. 4A. Corresponding sequences are shown below. Negative stain EM shows that the F ectodomain maintains the prefusion conformation, with the three HN ectodomains (one linked to each F protomer) arrayed C-terminal to the trimerization domain. This design yielded approximately 0.3 mg/L from Expi293 cells and was monodispersed on size exclusion chromatography and displayed expected assembly from negative-stain EM (FIG. 1C and FIG. 4A).

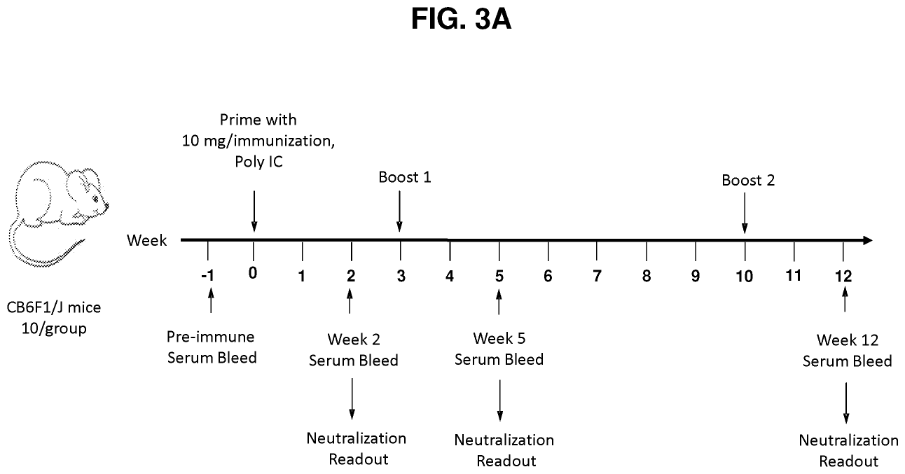

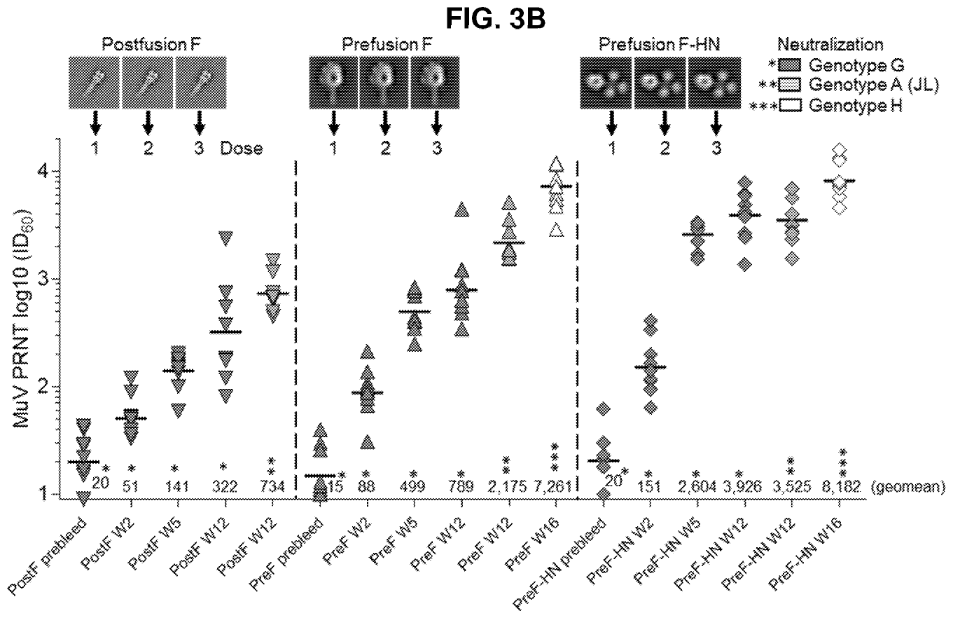

Prefusion-stabilized mumps F and prefusion-stabilized F-HN chimeric trimers elicit high-titer neutralizing antibodies in mice. The ability of prefusion-stabilized mumps F to elicit neutralizing antibodies compared to other mumps immunogens was assessed. Groups of 10 Cβ6F₁/J mice were immunized with 10 μg doses of mumps glycoproteins combined with 10 g polyinosinic-polycytidylic acid (poly-I:C) adjuvant at weeks 0, 3 and 10 and the ability of sera to prevent mumps virus infection of HEp-2 cells was measured (FIG. 3A).

The immunogens assessed were a MuV F ectodomain trimer in a post-fusion conformation (native ectodomain with-476-GCN4), a MuV F ectodomain trimer in a pre-fusion conformation (MuV F V206C-A223C-GGG-476-GCN4, SEQ ID NO: 11), MuV HN ectodomain monomer, and a MuV F ectodomain trimer in a pre-fusion conformation with the protomers of the trimer fused to MuV HN ectodomain (MuV F 206C-223C-GGG-476+GCN4+MuV HN_G (SEQ ID NO: 27).

Where mice were immunized with prefusion mumps F-containing immunogens (preF or preF-HN), specific responses to preF were detected in sera, with lower levels of binding to postfusion-immunized mice (FIG. 4B). Recombinant HN monomer bound only HN-immunized mouse sera, however monomeric HN immunized mice showed little binding whereas sera from preF-HN immunized mice showed substantial levels of HN binding suggesting multivalency of HN was driving a robust humoral responses (FIG. 4C).

To analyze the elicitation of neutralizing antibody titers from three immunizations with either postfusion F, prefusion F or prefusion F-HN, the PRNT 2 weeks following each immunization was analyzed (FIG. 3B). Incremental increases in neutralizing titers were observed after each immunization, however the third immunization showed a more marginal increase than after the second immunization. Neutralizing antibodies were observed with postfusion immunization, however the prefusion F immunogen showed a 3.5 and 2.5-fold higher neutralization than postfusion F after the second and third immunization respectively (geometric mean infective dose (ID₆₀ values) of 322 and 789 respectively to genotype G mumps virus). The preF-HN chimeric variant elicited neutralizing titers after the third immunization 12-fold higher than postfusion F and 5-fold higher than prefusion F, with an ID₆₀ value of 3930 to genotype G virus.

Next, the PRNT to Jeryl Lynn and genotype H viruses was evaluated to characterize cross-neutralizing antibodies elicited from the recombinant immunogens. For postfusion and prefusion F, a greater level of neutralization was observed for Jeryl Lynn virus than to genotype G virus, whereas for the prefusion F-HN chimera equivalent PRNTs were observed. At week 16, sera from both preF and preF-HN groups showed robust PRNT to genotype H virus. This indicates these recombinant immunogens can elicit antibodies that can cross-neutralize numerous mumps genotypes. the durability of PRNT to these three mumps viruses was monitored for an additional 6 months and it was found that while there is an a reduction in titers, ID₆₀ plateaus form after 3 months and the preF-HN had geometric mean PRNTs for genotype G, Jeryl Lynn and genotype H viruses of approximately 640, 800 and 1700 respectively (FIG. 4D). The ID₆₀ PRNT plateaus formed with the preF immunogen against genotype G, Jeryl Lynn and genotype H viruses were approximately 100, 660 and 3153 (FIG. 4E). Overall, prefusion stabilized F mumps linked to HN yielded neutralization titers that were higher than F alone and represents a design strategy to combine both viral surface antigens in a single immunogen stoichiometrically.

### Discussion

Human immunity to mumps following MMR vaccination is characterized by PRNT typically around 220 for Jeryl Lynn and 40 for genotype G (Rasheed et al., Proc Nat Acad Sci USA 116(38):19071-19076, 2019). The results provided herein for the disclosed immunogens appear to provide increased effectiveness as measured by PNRT, assuming that the mouse model data correlated with response in a human.

Due to mumps outbreaks among two-dose vaccine recipients, improvements are needed to the current mumps vaccine to reduce disease incidence and the burden on public health resources. A third dose of mumps-containing vaccine has been recommended by the Advisory Committee on Immunization Practices (ACIP) to individuals at risk of contracting mumps in an outbreak setting. A third dose of live attenuated MMR has resulted in a temporary elevation of neutralization titers, lasting about 12 months (Fiebelkorn A. P. et al. Open Forum Infect Dis 1(3):ofu094, 2014). Additionally, there was a 78% reduced risk of contracting mumps infection after a third dose of MMR compared to individuals who had received two doses of MMR (Cardemil C. et al, Effectiveness of a Third Dose of MMR Vaccine for Mumps Outbreak Control. N Engl J Med. 377 (10): 947-956, 2017). The recombinant protein vaccine candidates described in this example provide an alternative vaccine modality than administration of MMR in a mumps outbreak setting and provide increased durability and effectiveness.

The prefusion F-HN chimera, comprising the two key neutralization targets on mumps virions is capable of eliciting potent cross-genotype neutralizing responses, including to the dominant mumps genotype G causing outbreaks and two other genotypes, A and H represents a universal vaccine candidate for global mumps strains.

Sequences:
MuV F A163C/V235C-GGG-483-GCN4
(SEQ ID NO: 2)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laCavqaiqdhintimntqlnnmscqildnqlatslglyltelttvfqpqltnpalspisiqalrsilgsmtpaCvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyikesnhqlqsvsvn
SkiIEDKIEEILSKIYHIENEIARIKKLIGEAP
MuV F V206C/A223C-GGG-483-GCN4
(SEQ ID NO: 3)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttCfqpqltnpalspisiqClrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltisisqtintqpidistelskvnaslqnavkyikesnhqlqsvsvn
SkiIEDKIEEILSKIYHIENEIARIKKLIGEAP
MuV F N86C/A215C-GGG-483-GCN4
(SEQ ID NO: 4)
mkafsvtclsfavfsssicvninilqqiqyikqqvrqlsyysqssssyivvkllpniqptddscefksvtqynktlsnll
lpiaeCinniaspspgsrrhGGGagiaigiaalgvataaqvtaavslvqaqtnaraiaamknsiqatnravfevkegtqq
laiavqaiqdhintimntqlnnmscqildnqlatslglyltelttvfqpqltnpClspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltisisqtintqpidistelskvnaslqnavkyikesnhqlqsvsvn
SkiIEDKIEEILSKIYHIENEIARIKKLIGEAP
MuV F P209C/P214C-GGG-483-GCN4
(SEQ ID NO: 5)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttvfqCqltnCalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltisisqtintqpidistelskvnaslqnavkyikesnhqlqsvsvn
SkiIEDKIEEILSKIYHIENEIARIKKLIGEAP
MuV F K155C/L161C-GGG-483-GCN4
(SEQ ID NO: 6)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevCegtqq
Caiavqaiqdhintimntqlnnmscqildnqlatslglyltelttvfqpqltnpalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltisisqtintqpidistelskvnaslqnavkyikesnhqlqsvsvn
SkiIEDKIEEILSKIYHIENEIARIKKLIGEAP
MuV F V165C/M231C-GGG-483-GCN4
(SEQ ID NO: 7)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiaCqaiqdhintimntqlnnmscqildnqlatslglyltelttvfqpqltnpalspisiqalrsllgsCtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltisisqtintqpidistelskvnaslqnavkyikesnhqlqsvsvn
SkiIEDKIEEILSKIYHIENEIARIKKLIGEAP
MuV F I221C/M255C-GGG-483-GCN4
(SEQ ID NO: 8)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttvfqpqltnpalspisCqalrsllgsmtpavvqatl
stsisaaeilsaglCegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltisisqtintqpidistelskvnaslqnavkyikesnhqlqsvsvn
SkiIEDKIEEILSKIYHIENEIARIKKLIGEAP
MuV F S184P/GGG-483-GCN4
(SEQ ID NO: 9)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmPcqildnqlatslglyltelttvfqpqltnpalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltisisqtintqpidistelskvnaslqnavkyikesnhqlqsvsvn
SkiIEDKIEEILSKIYHIENEIARIKKLIGEAP
MuV F A163C/V235C-GGG-476-GCN4
(SEQ ID NO: 10)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laCavqaiqdhintimntqlnnmscqildnqlatslglyltelttvfqpqltnpalspisiqalrsllgsmtpaCvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyikesnhqlqs
IEDKIEEILSKIYHIENEIARIKKLIGEAP
MuV F V206C/A223C-GGG-476-GCN4
(SEQ ID NO: 11)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttCfqpqltnpalspisiqClrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyikesnhqlqs
IEDKIEEILSKIYHIENEIARIKKLIGEAP
MuV F N86C/A215C-GGG-476-GCN4
(SEQ ID NO: 12)
mkafsvtclsfavfsssicvninilqqigyikqqvrqlsyysqssssyivvkllpniqptddscefksvtqynktlsnll
lpiaeCinniaspspgsrrhGGGagiaigiaalgvataaqvtaavslvqaqtnaraiaamknsiqatnravfevkegtqq
laiavqaiqdhintimntqlnnmscqildnqlatslglyltelttvfqpqltnpClspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyikesnhqlqs
IEDKIEEILSKIYHIENEIARIKKLIGEAP
MuV F P209C/P214C-GGG-476-GCN4
(SEQ ID NO: 13)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttvfqCqltnCalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyikesnhqlqs
IEDKIEEILSKIYHIENEIARIKKLIGEAP
MuV F K155C/L161C-GGG-476-GCN4
(SEQ ID NO: 14)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevCegtqq
Caiavqaiqdhintimntqlnnmscqildnqlatslglyltelttvfqpqltnpalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyikesnhqlqs
IEDKIEEILSKIYHIENEIARIKKLIGEAP
MuV F V165C/M231C-GGG-476-GCN4
(SEQ ID NO: 15)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiaCqaiqdhintimntqlnnmscqildnqlatslglyltelttvfqpqltnpalspisiqalrsiIgsCtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyikesnhqlqs
IEDKIEEILSKIYHIENEIARIKKLIGEAP
MuV F 1221C/M255C-GGG-476-GCN4
(SEQ ID NO: 16)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttvfqpqltnpalspisCqalrsllgsmtpavvqatl
stsisaaeilsaglCegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltisisqtintqpidistelskvnaslqnavkyikesnhqlqs
IEDKIEEILSKIYHIENEIARIKKLIGEAP
MuV F S184P-GGG-476-GCN4
(SEQ ID NO: 17)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmPcqildnqlatslglyltelttvfqpqltnpalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyikesnhqlqs
IEDKIEEILSKIYHIENEIARIKKLIGEAP
MuV F A163C/V235C-GGG-469-GCN4
(SEQ ID NO: 18)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laCavqaiqdhintimntqlnnmscqildnqlatslglyltelttvfqpqltnpalspisiqalrsllgsmtpaCvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyike
IEDKIEEILSKIYHIENEIARIKKLIGEAP
MuV F V206C/A223C-GGG-469-GCN4
(SEQ ID NO: 19)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttCfqpqltnpalspisiqClrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyike
IEDKIEEILSKIYHIENEIARIKKLIGEAP
MuV F N86C/A215C-GGG-469-GCN4
(SEQ ID NO: 20)
mkafsvtclsfavfsssicvninilqqiqyikqqvrqlsyysqssssyivvkllpniqptddscefksvtqynktlsnll
lpiaeCinniaspspgsrrhGGGagiaigiaalgvataaqvtaavslvqaqtnaraiaamknsiqatnravfevkegtqq
laiavqaiqdhintimntqlnnmscqildnqlatslglyltelttvfqpqltnpClspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyike
IEDKIEEILSKIYHIENEIARIKKLIGEAP
MuV F P209C/P214C-GGG-469-GCN4
(SEQ ID NO: 21)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttvfqCqltnCalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyike
IEDKIEEILSKIYHIENEIARIKKLIGEAP
MuV F K155C/L161C-GGG-469-GCN4
(SEQ ID NO: 22)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevCegtqq
Caiavqaiqdhintimntqlnnmscqildnqlatslglyltelttvfqpqltnpalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyike
IEDKIEEILSKIYHIENEIARIKKLIGEAP
MuV F V165C/M231C-GGG-469-GCN4
(SEQ ID NO: 23)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiaCqaiqdhintimntqlnnmscqildnqlatslglyltelttvfqpqltnpalspisiqalrsilgsCtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltisisqtintqpidistelskvnaslqnavkyike
IEDKIEEILSKIYHIENEIARIKKLIGEAP
MuV F I221C/M255C-GGG-469-GCN4
(SEQ ID NO: 24)
mkafsvtclsfavfsssicvninilqqiqyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttvfqpqltnpalspisCqalrsllgsmtpavvqatl
stsisaaeilsaglCegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyike
IEDKIEEILSKIYHIENEIARIKKLIGEAP
MuV F S184P-GGG-469-GCN4
(SEQ ID NO: 25)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmPcqildnqlatslglyitelttvfqpqltnpalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyike
IEDKIEEILSKIYHIENEIARIKKLIGEAP
MuV-JL F 206C/A223C-GGG-476-GCN4
(SEQ ID NO: 26)
mkafsvtclsfavfsssicvninilqqiqyikqqvrqlsyysqssssyVvvkllpniqptdNscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttCfqpqllnpalspisiqClrsllgsmtpavvqatl
stsisaaeilsaglmegqiVsvlldemqmivkinVptivtqsnalvidfysissfinnqesiiqlpdrileigneqwRyp
aknckStrhhifcqyneaerisleTklclagnisacvfspiagsymrrfvaldgtivancrsltclckspsypiyqpdhh
avttidltScqtlsldgldfsivslsnityaenltislsqtintqpidistelskvnaslqnavkyikesnhqlqs
IEDKIEEILSKIYHIENEIARIKKLIGEAP
MuV-IL17 F 206C/A223C-GGG-476-GCN4
(SEQ ID NO: 51
mkvslvtclgfavfsfsicvninilqqiqyikqqvrqlsyysqssssyivvkllpniqptdnscefksvtqynktlsnll
lpiaeninniaspspgsrrhGGGagiaigiaalgvataaqvtaavslvqaqtnaraiaamknsiqatnravfevkegtqq
laiavqaiqnhintimntqlnnmscqildnqlatslglylteLTTCfqpqlinpalspisiqCLRSllgsmtpavvqatl
stsisaaeilsaglmegqivsvlldemqmivkiniptivtqsnalvidfysissfingqesiiqlpdrileigneqwsyp
aknckltrhnifcqyneaerlslesklclagnisacvfspiagsymrrfvaldgtivancrsltclckspsypiyqpdhh
avttidltacqtlsldgldfsivslsnityaenltisisqtintqpidistelikvnaslqnavkyikesnhqlqs
IEDKIEEILSKIYHIENEIARIKKLIGEAP
Postfusion MuV F ectodomain
(SEQ ID NO: 52)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevCegtqq
Caiavqaiqdhintimntqlnnmscqildnqlatslglyltelttvfqpqltnpalspisiqalrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyikesnhqlqsvsvn
skigailEDKIEEILSKIYHIENEIARIKKLIGEAP
MuV F 206C/223C-GGG-476 + GCN4 + MuV HN_G
(SEQ ID NO: 27)
mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttCfqpqltnpalspisiqClrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltisisqtintqpidistelskvnaslqnavkyikesnhqlqs
IEDKIEEILSKIYHIENEIARIKKLIGEAPGSGGGGGG
niplvndlrfinginkfiiedyathdfsighpinmpsfiptatspngctripsfslgkthwcythnvinanckdhtssnq
yvsmgilvqtasgypmfktikiqylsdglnrkscsiatvpdgcamycyvstqletddyagsspptqkltlifyndtvter
tispsglegnwatlvpgvgsgiyfenklifpayggvlpnstigvksareffrpvnpynpcsgpqqdldqralrsyfpsyf
snrriqsaflvcawnqilvtncelvvpssnqtmmgaegrvllinnrllyyqrstswwpyellyeisftftnsgpssvnms
wipiysftrpgsgncsgenvcptacvsgvyldpwpltpyshqsginrnfyftgallnssttrvnptlyvsalnnlkvlap
ygtqglfasyttttcfqdtgdasvycvyimelasnivgefqilpvltritit

The above sequences include an N-terminal signal peptide, a MuV F ectodomain, a GCN4 trimerization domain, optionally a MuV HN ectodomain, a thrombin cleavage site, a HIS tag and a Strep tag, as well as various linker residues between segments.

### Example 2

### MeV F Proteins Stabilized in a Prefusion Conformation and Fusions Thereof with MeV H Ectodomain or MuV HN Ectodomain

The example illustrates embodiments of a MeV F ectodomain trimer stabilized in a prefusion conformation by one or more amino acid substitutions. Further provided are MeV F ectodomain trimers linked to a MeV H ectodomain. The prefusion-stabilized MeV F ectodomain trimers and corresponding fusions with MeV H ectodomain are useful, for example, for inducing a neutralizing immune response to MeV in a subject.

When produced in cells, the MeV F ectodomain linked to a C-terminal GCN4 trimerization domain forms trimers that spontaneously transition to the prefusion conformation. Unstabilized recombinant MeV F-GCN4 is so unstable, 100% of molecules have transitioned to the postfusion conformation at the point of evaluation (EM). Also protein expression is substantially reduced without stabilization.

Accordingly, structure-based vaccine design was used to identify mutations for the stabilization of the MeV F ectodomain in a prefusion conformation (based on prefusion PIV5 F structure PDB ID 4WSG and MeV F structure PDB ID 5YXW), and also to eliminate the F1/F2 cleavage site to produce a “single chain” MeV F protein with increased expression. Multiple stabilization strategies were employed to “lock” the MeV F ectodomain in the prefusion conformation, including introduction of disulfide bonds and proline substitutions. In total, approximately 40 different mutants were designed, expressed, purified, assessed for expression level, and assessed for prefusion conformation by negative stain EM.

The mutations were introduced into a MeV F ectodomain (based on C-terminal truncations at MeV F position 486, and linked to a C-terminal GCN4 trimerization domain, and the resulting mutants were screened as noted above. The ectodomain also included a mutation to remove the F1/F2 furin cleavage site. The prefusion stabilizing mutations assessed included: cysteine substitutions at one or more of MeV F positions 48 and 284, 90 and 225, 141 and 270, 165 and 171, 173 and 245, 175 and 241, 212 and 236, 216 and 233, and 219 and 224 that form a non-natural disulfide bond; and a proline substitution at MeV F position 194. Relevant sequences are shown below.

The expression and purification of the single chain and prefusion-stabilized MeV F proteins showed a substantial increase in expression level compared to the unmodified MeV F.

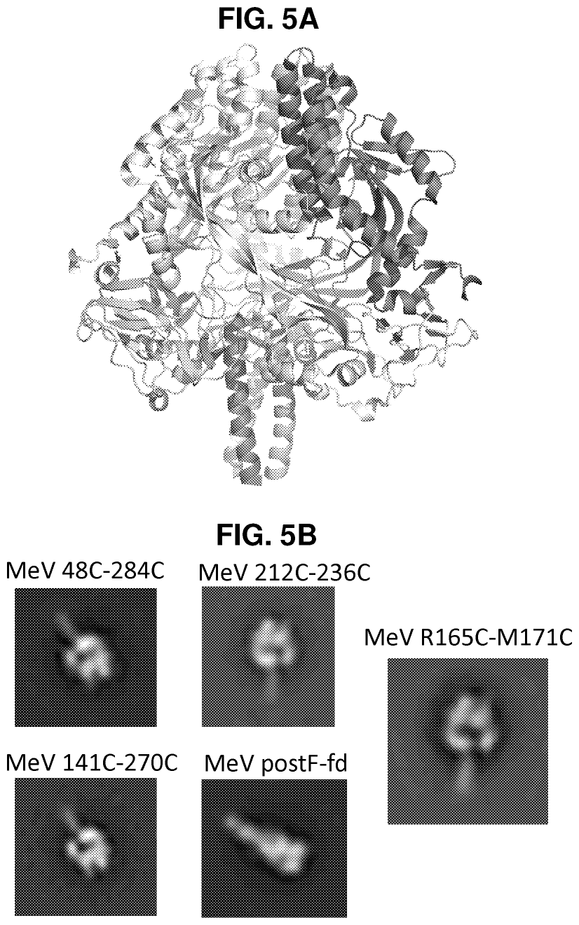

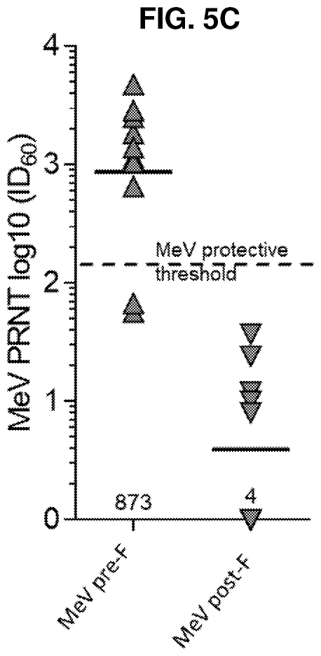

As illustrated in FIG. 5, negative EM can be used to distinguish MeV F ectodomain trimers that are in the prefusion conformation from those that are in the postfusion conformation. Also, MeV F R165C-M171C-486-GCN4 (SEQ ID NO: 38) showed an excellent combination of prefusion stabilization and protein expression, and purified as a monodispersed protein by S200 gel filtration. This construct and others were analyzed by electron microscopy to further confirm the prefusion conformation.

Immunization assays were conducted with a MeV F ectodomain trimer in a post-fusion conformation and a MeV F ectodomain trimer in a pre-fusion conformation (MeV F R165C-M171C-486-GCN4 (SEQ ID NO: 38). The immunization protocol was according to that shown in FIG. 3A. Groups of 10 Cβ6F1/J mice were immunized with 10 μg/dose of protein in Poly IC adjuvant at weeks 0 and 3, and the neutralization titer of week 5 sera from the immunized mice were assessed. The immune sera were assessed in a MeV neutralization assay (FIG. 5C), which showed that immune sera from animals immunized with the MeV F R165C-M171C-486-GCN4 (SEQ ID NO: 38) neutralized MeV more than 200-fold higher than sera from animals immunized with the postfusion MeV F, and above a protective threshold.

Sequences:
MeV F A90C/I225C-GGG-486-GCN4
(SEQ ID NO: 37)
mysmqlascvtltlvllvnsqihwgnlskigvvgigsasykvmtrsshqslviklmpnitllnnctrveiaeyrrllrtv
lepirdClnavtqnirpvqsvassrrhGGGagvvlagaalgvataaqitagialhqsmlnsqaidnlraslettnqaiea
irqagqemilavqgvqdyinnelipsmnqlscdligqklglkllryytellsifgpslrdpCsaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfiisqgnllaneaslickcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F R165C/M171C-GGG-486-GCN4
(SEQ ID NO: 38)
mysmqlascvtitlvllvnsqihwgnlskigvvgigsasykvmtrsshqslviklmpnitllnnctrveiaeyrrllrtv
lepirdalnavtqnirpvqsvassrrhGGGagvvlagaalgvataaqitagialhqsmlnsqaidnlraslettnqaiea
iCqagqeCilavqgvqdyinnelipsmnqlscdligqklglkllryyteilslfgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgniiancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F L173C/V245C-GGG-486-GCN4
(SEQ ID NO: 39)
mysmqlascvtltlvllvnsqihwgnlskiqvvgigsasykvmtrsshqslviklmpnitllnnctrveiaeyrrllrtv
lepirdalnavtqnirpvqsvassrrhGGGagvvlagaalgvataaqitagiaihqsmlnsqaidnlraslettnqaiea
irqagqemiCavqgvqdyinnelipsmnqlscdligqklglkllryytellsifgpslrdpisaeisiqalsyalggdin
kCleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F V175C/D241C-GGG-486-GCN4
(SEQ ID NO: 40)
mysmqlascvtltlvllvnsqihwqnlskiqvvqiqsasykvmtrsshqslviklmpnitllnnctrveiaeyrrllrtv
lepirdalnavtqnirpvqsvassrrhGGGagvvlagaaigvataaqitagiaihqsmlnsqaidnlraslettnqaiea
irqagqemilaCqgvqdyinnelipsmnqlscdligqklglkllryyteilslfgpslrdpisaeisiqalsyalggCin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F L216C/A233C-GGG-486-GCN4
(SEQ ID NO: 41)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaalgvataaqitagiaihqsmlnsqaidnlraslettnqaiea
irqagqemilavqgvqdyinnelipsmnqlscdligqklglkllryyteilsCfgpslrdpisaeisiqClsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F P219C/P224C-GGG-486-GCN4
(SEQ ID NO: 42)
mysmqlascvtltlvllvnsqihwgnlskiqvvgigsasykvmtrsshqslviklmpnitllnnctrveiaeyrrllrtv
lepirdalnavtqnirpvqsvassrrhGGGagvvlagaaigvataaqitagiaihqsmlnsqaidnlraslettnqaiea
irqagqemilavqgvqdyinnelipsmnqlscdligqklglkllryyteilslfgCslrdCisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F S194P-GGG-486-GCN4
(SEQ ID NO: 43)
mysmqlascvtltlvllvnsqihwgnlskiqvvgigsasykvmtrsshqslviklmpnitllnnctrveiaeyrrllrtv
lepirdalnavtqnirpvqsvassrrhGGGagvvlagaaigvataaqitagiaihqsmlnsqaidnlraslettnqaiea
irqagqemilavqgvqdyinnelipsmnqlPcdligqklglkllryyteilslfgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
Postfusion MeV F
(SEQ ID NO: 61)
MYSMQLASCVTLTLVLLVNSQIHWGNLSKIGVVGIGSASYKVMTRSSHQSLVIKLMPNITLLNNCTRVEIAEYRRLLRTV
LEPIRDALNAVTQNIRPVQSVASSGSGGGSAQITAGIALHQSMLNSQAIDNLRASLETTNQAIEAIRQAGQEMILAVQGV
QDYINNELIPSMNQLSCDLIGQKLGLKLLRYYTEILSLFGPSLRDPISAEISIQALSYALGGDINKVLEKLGYSGGDLLG
ILESRGIKARITHVDTESYFIVLSIAYPTLSEIKGVIVHRLEGVSYNIGSQEWYTTVPKYVATQGYLISNFDESSCTFMP
EGTVCSQNALYPMSPLLQECLRGSTKSCARTLVSGSFGNRFILSQGNLIANCASILCKCYTTGTIINQDPDKILTYIAAD
HCPVVEVNGVTIQVGSRRYPDAVYLHRIDLGPPISLERLDVGTNLGNAIAKLEDAKELLESSDQILSAIGGYIPEAPRDG
QAYVRKDGEWVLLSTFL
MeV F S103C/VH7C-GGG-486-GCN4
(SEQ ID NO: 62)
mysmqlascvtltlvllvnsqihwgnlskigvvgigsasykvmtrsshqslviklmpnitllnnctrveiaeyrrllrtv
lepirdalnavtqnirpvqCvassrrhGGGagvClagaaigvataaqitagiaihqsmlnsqaidnlraslettnqaiea
irqagqemilavqgvqdyinnelipsmnqlscdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F V175F-GGG-486-GCN4
(SEQ ID NO: 63)
mysmqlascvtltlvllvnsqihwgnlskiqvvgigsasykvmtrsshqslviklmpnitllnnctrveiaeyrrllrtv
lepirdalnavtqnirpvqsvassrrhGGGagvvlagaaigvataaqitagiaihqsmlnsqaidnlraslettnqaiea
irqagqemilaFqgvqdyinnelipsmnglscdligqklglkllryyteilslfgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F S103C/V117C, V175F, S194P-GGG-486-GCN4
(SEQ ID NO: 64)
mysmqlascvtitlvllvnsqihwgnlskigvvgigsasykvmtrsshqslviklmpnitllnnctrveiaeyrrllrtv
lepirdalnavtqnirpvqCvassrrhGGGagvClagaaigvataaqitagiaihqsminsqaidnlraslettnqaiea
irqagqemilaFqgvqdyinnelipsmnqlPcdligqklglkllryyteilslfgpslrdpisaeisiqalsyalggdin
kvleklqysqqdllqilesrgikarithvdtesyfivlsiayptlseikqvivhrlegvsynigsqewyttvpkyvatqq
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F S103C/V117C, R165C/M171C, S194P-GGG-486-GCN4
(SEQ ID NO: 65)
mysmqlascvtltlvllvnsqihwgnlskigvvgigsasykvmtrsshqslviklmpnitllnnctrveiaeyrrllrtv
lepirdalnavtqnirpvqCvassrrhGGGagvClagaalgvataaqitagiaihqsminsqaidnlraslettnqaiea
iCqagqeCilaFqgvqdyinnelipsmnqlPcdligqklglkllryytellsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F R99C/V117C-GGG-486-GCN4
(SEQ ID NO: 66)
mysmqlascvtitlvllvnsqihwgnlskigvvgigsasykvmtrsshqslviklmpnitllnnctrveiaeyrrllrtv
lepirdalnavtqniCpvqsvassrrhGGGagvClagaaigvataaqitagiaihqsminsqaidnlraslettnqaiea
irqagqemilavqgvqdyinnelipsmnqlscdligqklglkllryvteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F R99C/V117C, V175F, S194P-GGG-486-GCN4
(SEQ ID NO: 67)
mysmqlascvtitlvllvnsqihwgnlskigvvgigsasykvmtrsshqslviklmpnitllnnctrveiaeyrrllrtv
lepirdalnavtqniCpvqsvassrrhGGGagvClagaaigvataaqitagiaihqsminsqaidnlraslettnqaiea
irqagqemilaFqgvqdyinnelipsmnqlPcdligqklglkllryytellsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F R99C/V117C, R165C/M171C, S194P-GGG-486-GCN4
(SEQ ID NO: 68)
mysmqlascvtitlvllvnsqihwgnlskigvvgigsasykvmtrsshqslviklmpnitllnnctrveiaeyrrllrtv
lepirdalnavtqniCpvqsvassrrhGGGagvClagaalgvataaqitagialhqsminsqaidnlraslettnqaiea
iCqagqeCilaFqgvqdyinnelipsmnqlPcdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F R99C/V117C-GGG-493-GCN4
(SEQ ID NO: 69)
mysmqlascvtitlvllvnsqihwgnlskigvvgigsasykvmtrsshqslviklmpnitllnnctrveiaeyrrllrtv
lepirdalnavtqniCpvqsvassrrhGGGagvClagaalgvataaqitagialhqsminsqaidnlraslettnqaiea
irqagqemilavqgvqdyinnelipsmnqlscdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lrsmkglssaIEDKIEEILSKIYHIENEIARIKKLIGEAP
MeV F R99C/V117C, V175F, S194P-GGG-493-GCN4
(SEQ ID NO: 70)
mysmqlascvtltlvllvnsqihwqnlskiqvvqiqsasykvmtrsshqslviklmpnitllnnctrveiaeyrrllrtv
lepirdalnavtqniCpvqsvassrrhGGGagvClagaaigvataaqitagiaihqsminsqaidnlraslettnqaiea
irqagqemilaFqgvqdyinnelipsmnqlPcdligqklglkllryyteilslfgpslrdpisaeisiqalsyalggdin
kvleklqysqqdllqilesrgikarithvdtesyfivlsiayptlseikqvivhrlegvsynigsqewyttvpkyvatqq
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lrsmkglssaIEDKIEEILSKIYHIENEIARIKKLIGEAP
MeV F R99C/V117C, R165C/M171C, S194P-GGG-493-GCN4
(SEQ ID NO: 71)
mysmqlascvtitlvllvnsqihwgnlskigvvgigsasykvmtrsshqslviklmpnitllnnctrveiaeyrrllrtv
lepirdalnavtqniCpvqsvassrrhGGGagvClagaaigvataaqitagiaihqsminsqaidnlraslettnqaiea
iCqagqeCilaFqgvqdyinnelipsmnqlPcdligqklglkllryyteilsifgpslrdpisaeisiqalsyalggdin
kvleklqysqqdllqilesrgikarithvdtesyfivlsiayptlseikqvivhrlegvsynigsqewyttvpkyvatqq
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lrsmkglssaIEDKIEEILSKIYHIENEIARIKKLIGEAP
MeV F P100C/V117C, R165C/M171C, S194P-GGG-486-GCN4
(SEQ ID NO: 72)
mysmqlascvtltlvllvnsqihwgnlskigvvgigsasykvmtrsshqslviklmpnitllnnctrveiaeyrrllrtv
lepirdalnavtqnirCvqsvassrrhGGGagvClagaaigvataaqitagiaihqsminsqaidnlraslettnqaiea
iCqagqeCilaFqgvqdyinnelipsmnqlPcdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F V101C/V117C, R165C/M171C, S194P-GGG-486-GCN4
(SEQ ID NO: 73)
mysmqlascvtltlvllvnsqihwgnlskiqvvgigsasykvmtrsshqslviklmpnitllnnctrveiaeyrrllrtv
lepirdalnavtqnirpCqsvassrrhGGGagvClagaaigvataaqitagiaihqsminsqaidnlraslettnqaiea
iCqagqeCilaFqgvqdyinnelipsmnqlPcdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F Q102C/V117C, R165C/M171C, S194P-GGG-486-GCN4
(SEQ ID NO: 74)
mysmqlascvtltlvllvnsqihwgnlskiqvvgigsasykvmtrsshqslviklmpnitllnnctrveiaeyrrllrtv
lepirdalnavtqnirpvCsvassrrhGGGagvClagaaigvataaqitagiaihqsminsqaidnlraslettnqaiea
iCqagqeCilaFqgvqdyinnelipsmnqlPcdligqklglkllryyteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F R165C/M171C, M141C/T270C-GGG-486-GCN4
(SEQ ID NO: 75)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaaigvataaqitagialhqsClnsqaidnlraslettnqaiea
iCqagqeCllavqgvqdyinnelipsmnqlscdligqklglkllryyteilsifgpslrdpisaeisiqalsyalggdin
kvleklgysggdllgilesrgikarichvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F R165C/M171C, E212C/Y236C-GGG-486-GCN4 (SEQ ID NO: 76)
mysmqlascvtltlvllvnsqihwgnlskiqvvgigsasykvmtrsshqslviklmpnitllnnctrveiaeyrrllrtv
lepirdalnavtqnirpvqsvassrrhGGGagvvlagaaigvataaqitagiaihqsminsqaidnlraslettnqaiea
iCqagqeCHavqgvqdyinnelipsmnqlscdligqklglkllryytCllslf gpslrdpisaeisiqalsCalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F R165C/M171C, R48C/A284C-GGG-486-GCN4
(SEQ ID NO: 77)
mysmqlascvtltlvllvnsqihwgnlskiqvvgigsasykvmtCsshqslviklmpnitllnnctrveiaeyrrllrtv
lepirdalnavtqnirpvqsvassrrhGGGagvvlagaalgvataaqitagialhqsmlnsqaidnlraslettnqaiea
iCqagqeCilavqgvqdyinnelipsmnqlscdligqklglkllryyteilsifgpslrdpisaeisiqalsyalggdin
kvleklgysggdllgilesrgikarithvdtesyfivlsiCyptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppisleridvgtnlgnaiakledakellessdqi
lsaIEDKIEEILSKIYHIENEIARIKKLIGEAP
MeV F R99C/V117C, native furin-486-GCN4
(SEQ ID NO: 78)
mysmqlascvtitivllvnsqihwqnlskiqvvqiqsasykvmtrsshqslviklmpnitllnnctrveiaeyrrllrtv
lepirdalnavtqniCpvqsvassrrhkrfagvClagaalgvataaqitagialhqsmlnsqaidnlraslettnqaiea
irqagqemilavqgvqdyinnelipsmnqlscdligqklglkllrvvteilsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F R99C/V117C, V175F, S194P, native furin-486-GCN4
(SEQ ID NO: 79)
mysmqlascvtltlvllvnsqihwqnlskiqvvqiqsasykvmtrsshqslviklmpnitllnnctrveiaeyrrllrtv
lepirdalnavtqniCpvqsvassrrhkrfagvClagaalgvataaqitagialhqsmlnsqaidnlraslettnqaiea
irqagqemilaFqgvqdyinnelipsmnqlPcdligqklglkllryytellsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP
MeV F R99C/V117C, R165C/M171C, S194P, native furin-486-GCN4
(SEQ ID NO: 80)
mysmqlascvtitivllvnsqihwgnlskigvvgigsasykvmtrsshqslviklmpnitllnnctrveiaeyrrllrtv
lepirdalnavtqniCpvqsvassrrhkrfagvClagaalgvataaqitagialhqsmlnsqaidnlraslettnqaiea
lCqagqeCilaFqgvqdyinnelipsmnqlPcdligqklglkllryytellsifgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAP

The above sequences include an N-terminal signal peptide, a MeV F ectodomain, a GCN4 trimerization domain, as well as various linker residues between segments.

Additionally, chimeric constructs with the MeV F ectodomain with amino acid substitutions for stabilization in a prefusion conformation linked to a MuV HN ectodomain or a MeV H ectodomain were designed as follows:

MeV F R165C/M171C-GGG-486-GCN4 + MeV_H
(SEQ ID NO: 56)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaalgvataaqitagialhqsmlnsqaidnlraslettnqaiea
iCqagqeCilavqgvqdyinnelipsmnqlscdligqklglkllryyteilslfgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAPGSGGGGGG
flavskgncsgpttirgqfsnmsislldlylgrgynvssivtmtsqgmyggtyivekpnlsskrselsqlsmyrvfevgv
irnpglgapvfhmtnyleqpvsndlsncmvalgelklaalchgedsitipyqgsgkgvsfqlvklgvwkspadmqswvpl
stddpvidrlyisshrgviadnqakwavpttrtddklrmetcfqqackgkiqticenpewaplkdnripsygvlsvdlsl
tvelkikiasgfgplithgsgmdlyksnhnnvywltippmknlalgvintlewiprfkvspylftvpikeagedchapty
ipaevdgdvklssnlvilpgqdlqyvlatydtsrvehavvyyvyspgrsfsyfypfripikgvpielqvecftwdqklwc
rhfcvladsesgghithsgmvgmgvsctvtredgtnrr
MeV F R165C/M171C-GGG-486-GCN4 + MuV_HN
(SEQ ID NO: 57)
mysmqlascvtltlvllvnsqihwgnlskigvvgigsasykvmtrsshqslviklmpnitllnnctrveiaeyrrllrtv
lepirdalnavtqnirpvqsvassrrhGGGagvvlagaalgvataaqitagialhqsmlnsqaidnlraslettnqaiea
iCqagqeCilavqgvqdyinnelipsmnqlscdligqklglkllryyteilslfgpslrdpisaeisiqalsyalggdin
kvleklgysggdllgilesrgikarithvdtesyfivlsiayptlseikgvivhrlegvsynigsqewyttvpkyvatqg
ylisnfdessctfmpegtvcsqnalypmspllqeclrgstkscartlvsgsfgnrfilsqgnliancasilckcyttgti
inqdpdkiltyiaadhcpvvevngvtiqvgsrrypdavylhridlgppislerldvgtnlgnaiakledakellessdqi
lsaIEDKIEEILSKIYHIENEIARIKKLIGEAPGsgggggg
niplvndlrfinginkfiiedyathdfsighpinmpsfiptatspngctripsfsigkthwcythnvinanckdhtssnq
yvsmgilvqtasgypmfktikiqylsdglnrkscsiatvpdgcamycyvstqletddyagsspptqkltllfyndtvter
tispsglegnwatlvpgvgsgiyfenklifpayggvlpnstigvksareffrpvnpynpcsgpqqdldqralrsyfpsyf
snrriqsaflvcawnqilvtncelvvpssnqtmmgaegrvllinnrllyyqrstswwpyellyeisftftnsgpssvnms
wipiysftrpgsgncsgenvcptacvsgvyldpwpltpyshqsginrnfyftgallnssttrvnptlyvsalnnlkvlap
ygtqglfasyttttcfqdtgdasvycvyimelasnivgefqilpvltritit
MeV F R165C/M171C-GGG-486-GCN4/Fd + MeV_H
(SEQ ID NO: 81)
MYSMQLASCVTLTLVLLVNSQIHWGNLSKIGVVGIGSASYKVMTRSSHQSLVIKLMPNITLLNNCTRVEIAEYRRLLRTV
LEPIRDALNAVTQNIRPVQSVASSRRHGGGAGVVLAGAALGVATAAQITAGIALHQSMLNSQAIDNLRASLETTNQAIEA
ICQAGQECILAVQGVQDYINNELIPSMNQLSCDLIGQKLGLKLLRYYTEILSLFGPSLRDPISAEISIQALSYALGGDIN
KVLEKLGYSGGDLLGILESRGIKARITHVDTESYFIVLSIAYPTLSEIKGVIVHRLEGVSYNIGSQEWYTTVPKYVATQG
YLISNFDESSCTFMPEGTVCSQNALYPMSPLLQECLRGSTKSCARTLVSGSFGNRFILSQGNLIANCASILCKCYTTGTI
INQDPDKILTYIAADHCPVVEVNGVTIQVGSRRYPDAVYLHRIDLGPPISLERLDVGTNLGNAIAKLEDAKELLESSDQI
LSAIEDKIEEILSKIYHIENEIARIKKLIGEAPGSGYIPEAPRDGQAYVRKDGEWVLLSTFLGSGGGGGgFLAVSKGNCS
GPTTIRGQFSNMSLSLLDLYLGRGYNVSSIVTMTSQGMYGGTYLVEKPNLSSKRSELSQLSMYRVFEVGVIRNPGLGAPV
FHMTNYLEQPVSNDLSNCMVALGELKLAALCHGEDSITIPYQGSGKGVSFQLVKLGVWKSPADMQSWVPLSTDDPVIDRL
YLSSHRGVIADNQAKWAVPTTRTDDKLRMETCFQQACKGKIQTLCENPEWAPLKDNRIPSYGVLSVDLSLTVELKIKIAS
GFGPLITHGSGMDLYKSNHNNVYWLTIPPMKNLALGVINTLEWIPRFKVSPYLFTVPIKEAGEDCHAPTYLPAEVDGDVK
LSSNLVILPGQDLQYVLATYDTSRVEHAVVYYVYSPGRSFSYFYPFRLPIKGVPIELQVECFTWDQKLWCRHFCVLADSE
SGGHITHSGMVGMGVSCTVTREDGTNRR

The above sequences include an N-terminal signal peptide, a MeV F ectodomain, a GCN4 trimerization domain, optionally a T4 Fibritin trimerization domain, a MeV H ectodomain, as well as various linker residues between segments.

### Example 3

### Multimeric MuV HN, Multimeric MeV H, and MuV Pre-F-MeV H Chimera

This example describes an embodiment of a recombinant MuV pre-F ectodomain trimer linked to MeV H ectodomain to provide a chimeric immunogen that elicits a cross-neutralizing immune response to MeV and MuV. Additionally, multimeric MuV HN and multimeric MeV H are described.

To increase the immunogenic footprint of the MuV F ectodomain trimer, a MeV H ectodomain was genetically fused to the C-terminus of the trimerization domain of each protomer of the trimer. The assessed construct included a MuV F ectodomain containing V206C-A223C, a mutation to eliminate the F1/F2 furin cleavage site, a trimerization domain fused to position 476 of the ectodomain, and a MeV H ectodomain linked to the C-terminus of the trimerization domain. In this embodiment, the trimerization domain included both a GCN4 trimerization domain and a T4 fibritin trimerization domain in series; however, either of these domains can also be used on their own. The format is illustrated in FIG. 6A. A corresponding sequence is shown below.

MuV F 206C/223C-476 + GCN4/Fd + MeV-H (SEQ ID NO: 28):
Mkafsvtclsfavfsssicvninilqqigyikqqvrqlsyysqssssyivvkllpniqptddscefksvtqynktlsnll
lpiaeninniaspspgsrrhGGGagiaigiaalgvataaqvtaavslvqaqtnaraiaamknsiqatnravfevkegtqq
laiavqaiqdhintimntqlnnmscqildnqlatslglyltelttCfqpqltnpalspisiqClrsllgsmtpavvqatl
stsisaaeilsaglmegqiisvlldemqmivkiniptivtqsnalvidfysissfinnqesiiqlpdrileigneqwsyp
aknckltrhhifcqyneaerlslesklclagnisacvfspiagsymrrfvaldgtivancrsltclckspsypiyqpdhh
avttidltacqtlsldgldfsivslsnityaenltislsqtintqpidistelskvnaslqnavkyikesnhqlqs
IEDKIEEILSKIYHIENEIARIKKLIGEAPGSGYIPEAPRDGQAYVRKDGEWVLLSTFLGSGGGGGgFlavskgncsgpt
tirgqfsnmsislldlylgrgynvssivtmtsqgmyggtyivekpnlsskrselsqlsmyrvfevgvirnpglgapvfhm
tnyleqpvsndlsncmvalgelklaalchgedsitipyqgsgkgvsfqlvklgvwkspadmqswvplstddpvidrlyls
shrgviadnqakwavpttrtddklrmetcfqqackgkiqticenpewaplkdnripsygvlsvdlsltvelkikiasgfg
plithgsgmdlyksnhnnvywltippmknlalgvintlewiprfkvspylftvpikeagedchaptyipaevdgdvklss
nlvilpgqdlqyvlatydtsrvehavvyyvyspgrsfsyfypfripikgvpielqvecftwdqklwcrhfcvladsesgg
hithsgmvgmgvsctvtredgtnrr

Negative stain EM of purified MuV F 206C-223C-476+GCN4/Fd+MeV-H (SEQ ID NO: 28) shows that the F ectodomain maintains the prefusion conformation, with the three H ectodomains (one linked to each F protomer) arrayed C-terminal to the trimerization domain (FIG. 6A). The negative stain EM shows that this construct assembles with a conformation similar to MuV F-MuV HN descried in Example 1.

Additional immunogens were constructed containing multimers of the MeV H ectodomain head region, or the MuV HN ectodomain head region.

Trimeric MuV HN ectodomain head region and trimeric MeV H ectodomain head region were constructed by linking the N-terminus of the head region to a T4 fibritin trimerization domain. The sequences are provided as follows:

Fd-MuV HN
(trimeric MuV HN, SEQ ID NO: 58)
mysmqlascvtltlvllvnsqGSGYIPEAPRDGQAYVRKDGEWVLLSTFLGSGGGGGg
NIPLVNDLRFINGINKFIIEDYATHDFSIGHPLNMPSFIPTATSPNGCTRIPSFSLGKTHWCYTHNVINANCKDHTSSNQ
YVSMGILVQTASGYPMFKTLKIQYLSDGLNRKSCSIATVPDGCAMYCYVSTQLETDDYAGSSPPTQKLTLLFYNDTVTER
TISPSGLEGNWATLVPGVGSGIYFENKLIFPAYGGVLPNSTLGVKSAREFFRPVNPYNPCSGPQQDLDQRALRSYFPSYF
SNRRIQSAFLVCAWNQILVTNCELVVPSSNQTMMGAEGRVLLINNRLLYYQRSTSWWPYELLYEISFTFTNSGPSSVNMS
WIPIYSFTRPGSGNCSGENVCPTACVSGVYLDPWPLTPYSHQSGINRNFYFTGALLNSSTTRVNPTLYVSALNNLKVLAP
YGTQGLFASYTTTTCFQDTGDASVYCVYIMELASNIVGEFQILPVLTRLTIT
Fd-MeV H
(trimeric MeV H, SEQ ID NO: 59)
mysmqlascvtltlvllvnsqGSGYIPEAPRDGQAYVRKDGEWVLLSTFLGSGGGGGg
FLAVSKGNCSGPTTIRGQFSNMSLSLLDLYLGRGYNVSSIVTMTSQGMYGGTYLVEKPNLSSKRSELSQLSMYRVFEVGV
IRNPGLGAPVFHMTNYLEQPVSNDLSNCMVALGELKLAALCHGEDSITIPYQGSGKGVSFQLVKLGVWKSPADMQSWVPL
STDDPVIDRLYLSSHRGVIADNQAKWAVPTTRTDDKLRMETCFQQACKGKIQTLCENPEWAPLKDNRIPSYGVLSVDLSL
TVELKIKIASGFGPLITHGSGMDLYKSNHNNVYWLTIPPMKNLALGVINTLEWIPRFKVSPYLFTVPIKEAGEDCHAPTY
LPAEVDGDVKLSSNLVILPGQDLQYVLATYDTSRVEHAVVYYVYSPGRSFSYFYPFRLPIKGVPIELQVECFTWDQKLWC
RHFCVLADSESGGHITHSGMVGMGVSCTVTREDGTNRR

Dimeric MeV H was constructed by expressing the MeV H ectodomain head region in mammalian cells and purifying the resulting protein complex; the MeV H head dimerizes in physiological solution. The sequence of the MeV H head region is provided as follows:

Dimeric MeV H head region
(SEQ ID NO: 60)
Mysmqlascvtltlvllvnsqflavskgncsgpttirgqfsnmslslld
lylgrgynvssivtmtsqgmyggtylvekpnlsskrselsqlsmyrvfev
gvirnpglgapvfhmtnyleqpvsndlsncmvalgelklaalchgedsit
ipyqgsgkgvsfqlvklgvwkspadmqswvplstddpvidrlylsshrgv
iadngakwavpttrtddklrmetcfqqackgkiqtlcenpewaplkdnri
psygvlsvdlsltvelkikiasgfgplithgsgmdlyksnhnnvywltip
pmknlalgvintlewiprfkvspyiftvpikeagedchaptyipaevdgd
vklssnlvilpgqdlqyvlatydtsrvehavvyyvyspgrsfsyfypfri
pikgvpielqvecftwdqklwcrhfcvladsesgghithsgmvgmgvsct
vtredgtnrr

Dimeric MeV H including the stalk and head regions can be constructed by expressing the MeV H ectodomain stalk and head regions in mammalian cells and purifying the resulting protein complex; the MeV H stalk and head dimerizes in physiological solution. Exemplary sequences of MeV H stalk and head regions are provided as follows:

MeV H stalk and head
(SEQ ID NO: 86)
mysmqlascvtltlvllvnsqRLHraaiytaeihkslstnldvtnsiehqvkdvltplfkiiqdevqlrtpqrftdlv
kfisdkikfinpdreydfrdltwcinpperikldydqycadvaaeelmnalvnstlletrttnqFLAVskgncsgpttir
gqfsnmsislldlylgrgynvssivtmtsqgmyggtyivekpnlsskrselsqlsmyrvfevgvirnpglgapvfhmtny
leqpvsndlsncmvalgelklaalchgedsitipyqgsgkgvsfqlvklgvwksptdmqswvplstddpvidrlyisshr
gviadnqakwavpttrtddklrmetcfqqackgkiqalcenpewaplkdnripsygvlsvdlsltvelkikiasgfgpli
thgsgmdlyksnhnnvywltippmknlalgvintlewiprfkvspylfnvpikeagedchaptylpaevdgdvklssnlv
ilpgqdlqyvlatydtsrvehavvyyvyspsrsfsyfypfripikgvpielqvecftwdqklwcrhfcvladsesgghit
hsgmegmgvsctvtredgtnrr
MeV H stalk and head 2
(SEQ ID NO: 87)
mysmqlascvtltlvllvnsqraaiytaeihksistnldvtnsiehqvkdvltplfkiiqdevqlrtpqrftdivkfi
sdkikfinpdreydfrdltwcinpperikldydqycadvaaeelmnalvnstlletrttnqFLAVskgncsgpttirgqf
snmslslldlylgrgynvssivtmtsqgmyggtylvekpnlsskrselsqlsmyrvfevgvirnpglgapvfhmtnyleq
pvsndlsncmvalgelklaalchgedsitipyqgsgkgvsfqlvklgvwksptdmqswvplstddpvidrlylsshrgvi
adnqakwavpttrtddklrmetcfqqackgkiqalcenpewaplkdnripsygvlsvdlsitvelkikiasgfgplithg
sgmdlyksnhnnvywltippmknlaigvintlewiprfkvspylfnvpikeagedchaptyipaevdgdvkissnlviip
gqdiqyviatydtsrvehavvyyvyspsrsfsyfypfripikgvpielqvecftwdqklwcrhfcvladsesgghithsg
megmgvsctvtredgtnrr
MeV H stalk and head 3
(SEQ ID NO: 88)
mysmqlascvtltlvllvnsqLHraaiytaeihksistnldvtnsiehqvkdvltplfkligdevglrtpqrftdivk
fisdkikfinpdreydfrdltwcinpperikldydqycadvaaeelmnalvnstlletrttnqFLAVskgncsgpttirg
qfsnmslslldlylgrgynvssivtmtsqgmyggtylvekpnlsskrselsqlsmyrvfevgvirnpglgapvfhmtnyl
eqpvsndlsncmvalgelklaalchgedsitipyqgsgkgvsfqlvklgvwksptdmqswvplstddpvidrlylsshrg
viadnqakwavpttrtddklrmetcfqqackgkiqalcenpewaplkdnripsygvlsvdlsitvelkikiasgfgplit
hgsgmdlyksnhnnvywltippmknlalgvintlewiprfkvspylfnvpikeagedchaptylpaevdgdvkissnlvi
lpgqdlqyvlatydtsrvehavvyyvyspsrsfsyfypfripikgvpielqvecftwdqklwcrhfcvladsesgghith
sgmegmgvsctvtredgtnrr
MeV H stalk and head 4
(SEQ ID NO: 89)
mysmqlascvtltlvllvnsqtaeihkslstnldvtnsiehqvkdvltplfkligdevglrtpqrftdivkfisdkik
flnpdreydfrdltwcinpperikldydqycadvaaeelmnalvnstlletrttnqFLAVskgncsgpttirgqfsnmsl
slldlylgrgynvssivtmtsqgmyggtyivekpnlsskrselsqlsmyrvfevgvirnpglgapvfhmtnyleqpvsnd
lsncmvalgelklaalchgedsitipyqgsgkgvsfqlvklgvwksptdmqswvplstddpvidrlyisshrgviadnqa
kwavpttrtddklrmetcfqqackgkiqalcenpewaplkdnripsygvlsvdlsltvelkikiasgfgplithgsgmdl
yksnhnnvywltippmknlalgvintlewiprfkvspylfnvpikeagedchaptyipaevdgdvkissnlviipgqdlq
yvlatydtsrvehavvyyvyspsrsfsyfypfripikgvpielqvecftwdqklwcrhfcvladsesgghithsgmegmg
vsctvtredgtnrr

MuV HN including the stalk and head regions can be constructed by expressing the MuV HN ectodomain stalk and head regions in mammalian cells and purifying the resulting protein. Exemplary sequences of MuV HN stalk and head regions are provided as follows:

MuV HN stalk and head
(SEQ ID NO: 90)
mysmqlascvtltlvllvnsqELVRmindqglsnqlssitdkiresatmiasavgvmnqvihgvtvslplqiegnqnq
llatlatictsqkqvsncstNIPlvndlrfinginkfiiedyathdfsighpinmpsfiptatspngctripsfslgkth
wcythnvinanckdhtssnqyvsmgilvqtasgypmfktikiqylsdglnrkscsiatvpdgcamycyvstqletddyag
sspptqkltllfyndtvtertispsglegnwativpgvgsgiyfenklifpayggvlpnstigvksareffrpvnpynpc
sgpqqdldqralrsyfpsyfsnrriqsafivcawnqilvtncelvvpssnqtmmgaegrvllinnrllyyqrstswwpye
llyeisftftnsgpssvnmswipiysftrpgsgncsgenvcptacvsgvyidpwpltpyshqsginrnfyftgallnsst
trvnptlyvsalnnlkvlapygtqglfasyttttcfqdtgdasvycvyimelasnivgefqilpvltritit
MuV HN stalk and head 2
(SEQ ID NO: 91)
mysmqlascvtltlvllvnsqdqglsnqlssitdkiresatmiasavgvmnqvihgvtvslplqiegnqnqllatlat
ictsqkqvsncstNIPlvndlrfinginkfiiedyathdfsighpinmpsfiptatspngctripsfslgkthwcythnv
inanckdhtssnqyvsmgilvqtasgypmfktikiqylsdglnrkscsiatvpdgcamycyvstqletddyagsspptqk
itllfyndtvtertispsglegnwatlvpgvgsgiyfenklifpayggvlpnstlgvksareffrpvnpynpcsgpqqdl
dqrairsyfpsyfsnrriqsaflvcawnqilvtnceivvpssnqtmmgaegrvllinnrllyyqrstswwpyellyeisf
tftnsgpssvnmswipiysftrpgsgncsgenvcptacvsgvyidpwpltpyshqsginrnfyftgallnssttrvnptl
yvsalnnlkvlapygtqglfasyttttcfqdtgdasvycvyimelasnivgefqilpvltritit
MuV HN stalk and head 3
(SEQ ID NO: 92)
mysmqlascvtltlvllvnsqqlsnqlssitdkiresatmiasavqvmnqvihqvtvslplqieqnqnqllatlatic
tsqkqvsncstNIPlvndlrfinginkfiiedyathdfsighpinmpsfiptatspngctripsfslgkthwcythnvin
anckdhtssnqyvsmgilvqtasgypmfktikiqylsdglnrkscsiatvpdgcamycyvstqletddyagsspptqklt
llfyndtvtertispsglegnwatlvpgvgsgiyfenklifpayggvlpnstigvksareffrpvnpynpcsgpqqdldq
rairsyfpsyfsnrriqsaflvcawnqilvtnceivvpssnqtmmgaegrvllinnrllyyqrstswwpyellyeisftf
tnsgpssvnmswipiysftrpgsgncsgenvcptacvsgvyidpwpltpyshqsginrnfyftgallnssttrvnptlyv
salnnlkvlapygtqglfasyttttcfqdtgdasvycvyimelasnivgefqilpvltritit
MuV HN stalk and head 4
(SEQ ID NO: 93)
mysmqlascvtltlvllvnsqLVRmindqqlsnqlssitdkiresatmiasavqvmnqvihqvtvslplqieqnqnql
latlatictsqkqvsncstNIPlvndlrfinginkfiiedyathdfsighpinmpsfiptatspngctripsfslgkthw
cythnvinanckdhtssnqyvsmgilvqtasgypmfktikiqylsdglnrkscsiatvpdgcamycyvstqletddyags
spptqkltllfyndtvtertispsglegnwativpgvgsgiyfenklifpayggvlpnstlgvksareffrpvnpynpcs
gpqqdldqrairsyfpsyfsnrriqsafivcawnqlivtnceivvpssnqtmmgaegrvllinnrllyyqrstswwpyel
lyeisftftnsgpssvnmswipiysftrpgsgncsgenvcptacvsgvyidpwpltpyshqsginrnfyftgallnsstt
rvnptlyvsalnnlkvlapygtqglfasyttttcfqdtgdasvycvyimelasnivgefqilpvltritit

Additionally, chimeric versions of the trimeric MuV HN ectodomain head region and trimeric MeV H ectodomain head region were constructed by linking these molecules to the N- and C-termini of a T4 fibritin trimerization domain and/or GCN4 trimerization domain. The sequences are provided as follows:

MuV HN-Fd-MeV H
(SEQ ID NO: 82)
mysmqlascvtltlvllvnsqNIPlvndlrfinginkfiiedyathdfsighpinmpsfiptatspngctripsfsig
kthwcythnvinanckdhtssnqyvsmgilvqtasgypmfktikiqyisdglnrkscsiatvpdgcamycyvstqletdd
yagsspptqkltllfyndtvtertispsglegnwatlvpgvgsgiyfenklifpayggvlpnstlgvksareffrpvnpy
npcsgpqqdldqrairsyfpsyfsnrriqsafivcawnqilvtncelvvpssnqtmmgaegrvllinnrllyyqrstsww
pyellyeisftftnsgpssvnmswipiysftrpgsgncsgenvcptacvsgvyidpwpltpyshqsginrnfyftgalln
ssttrvnptlyvsalnnlkvlapygtqglfasyttttcfqdtgdasvycvyimelasnivgefqilpvltrltitgg
GSGYIPEAPRDGQAYVRKDGEWVLLSTFLGSGGGGGg
flavskgncsgpttirgqfsnmsislldlylgrgynvssivtmtsqgmyggtyivekpnlsskrselsqlsmyrvfevgv
irnpglgapvfhmtnyleqpvsndlsncmvalgelklaalchgedsitipyqgsgkgvsfqlvklgvwksptdmqswvpl
stddpvidrlyisshrgviadnqakwavpttrtddklrmetcfqqackgkiqalcenpewaplkdnripsygvlsvdlsl
tvelkikiasgfgplithgsgmdlyksnhnnvywltippmknlalgvintlewiprfkvspylfnvpikeagedchapty
lpaevdgdvklssnlvilpgqdlqyvlatydtsrvehavvyyvyspsrsfsyfypfripikgvpielqvecftwdqklwc
rhfcvladsesgghithsgmegmgvsctvtredgtnrr
MuV HN-GCN4/Fd-MeV H
(SEQ ID NO: 83)
mysmqlascvtltlvllvnsqNIPlvndlrfinginkfiiedyathdfsighpinmpsfiptatspngctripsfsig
kthwcythnvinanckdhtssnqyvsmgilvqtasgypmfktikiqyIsdglnrkscsiatvpdgcamycyvstqletdd
yagsspptqkltllfyndtvtertispsglegnwatlvpgvgsgiyfenklifpayggvlpnstlgvksareffrpvnpy
npcsgpqqdldqralrsyfpsyfsnrriqsaflvcawngilvtncelvvpssnqtmmgaegrvllinnrllyyqrstsww
pyellyeisftftnsgpssvnmswipiysftrpgsgncsgenvcptacvsgvyldpwpltpyshqsginrnfyftgalln
ssttrvnptlyvsalnnlkvlapygtqglfasyttttcfqdtgdasvycvyimelasnivgefqilpvltrltitgg
KLMKQIEDKIEEILSKIYHIENEIARIKKLIGEAPGSGYIPEAPRDGQAYVRKDGEWVLLSTFLGSGGGGGg
flavskgncsgpttirgqfsnmsislldlylgrgynvssivtmtsqgmyggtyivekpnlsskrselsqlsmyrvfevgv
irnpglgapvfhmtnyleqpvsndlsncmvalgeiklaalchgedsitipyqgsgkgvsfqlvklgvwksptdmqswvpl
stddpvidrlyisshrgviadnqakwavpttrtddklrmetcfqqackgkiqalcenpewaplkdnripsygvlsvdlsl
tvelkikiasgfgplithgsgmdlyksnhnnvywltippmknlalgvintlewiprfkvspylfnvpikeagedchapty
ipaevdgdvklssnlvilpgqdlqyvlatydtsrvehavvyyvyspsrsfsyfypfripikgvpielqvecftwdqklwc
rhfcvladsesgghithsgmegmgvsctvtredgtnrr
MeV H-Fd-MuV HN
(SEQ ID NO: 84)
mysmqlascvtltlvllvnsqFLAvskgncsgpttirgqfsnmslslldlylgrgynvssivtmtsqgmyggtylvek
pnlsskrselsqlsmyrvfevgvirnpglgapvfhmtnyleqpvsndlsncmvalgeiklaalchgedsitipyqgsgkg
vsfqlvklgvwksptdmqswvplstddpvidrlyisshrgviadnqakwavpttrtddklrmetcfqqackgkiqalcen
pewaplkdnripsygvlsvdlsitvelkikiasgfgplithgsgmdlyksnhnnvywltippmknlalgvintlewiprf
kvspylfnvpikeagedchaptylpaevdgdvklssnlvilpgqdlqyvlatydtsrvehavvyyvyspsrsfsyfypfr
ipikgvpielqvecftwdqklwcrhfcvladsesgghithsgmegmgvsctvtredgtnrr
GSGYIPEAPRDGQAYVRKDGEWVLLSTFLGSGGGGGg
NIPlvndlrfinginkfiiedyathdfsighpinmpsfiptatspngctripsfslgkthwcythnvinanckdhtssnq
yvsmgilvqtasgypmfktikiqylsdglnrkscsiatvpdgcamycyvstqletddyagsspptqkltlifyndtvter
tispsglegnwativpgvgsgiyfenklifpayggvlpnstigvksareffrpvnpynpcsgpqqdldqralrsyfpsyf
snrriqsaflvcawnqilvtncelvvpssnqtmmgaegrvllinnrllyyqrstswwpyellyeisftftnsgpssvnms
wipiysftrpgsgncsgenvcptacvsgvyldpwpltpyshqsginrnfyftgallnssttrvnptlyvsalnnlkvlap
ygtqglfasyttttcfqdtgdasvycvyimelasnivgefqilpvltrltit
MeV H-GCN4/Fd-MuV HN
(SEQ ID NO: 85)
mysmqlascvtltlvllvnsqFLAvskgncsgpttirgqfsnmslslldlylgrgynvssivtmtsqgmyggtylvek
pnlsskrselsqlsmyrvfevgvirnpglgapvfhmtnyleqpvsndlsncmvalgeiklaalchgedsitipyqgsgkg
vsfqlvklgvwksptdmqswvplstddpvidrlyisshrgviadnqakwavpttrtddklrmetcfqqackgkiqalcen
pewaplkdnripsygvlsvdlsltvelkikiasgfgplithgsgmdlyksnhnnvywltippmknlalgvintlewiprf
kvspylfnvpikeagedchaptyIpaevdgdvkissnlvilpgqdlqyvlatydtsrvehawyyvyspsrsfsyfypfr
lpikgvpielqvecftwdqklwcrhfcvladsesgghithsgmegmgvsctvtredgtnrr
MKQIEDKIEEILSKIYHIENEIARIKKLIGEAPGSGYIPEAPRDGQAYVRKDGEWVLLSTFLGSGGGGGg
NIPlvndlrfinginkfiiedyathdfsighpinmpsfiptatspngctripsfslgkthwcythnvinanckdhtssnq
yvsmgilvqtasgypmfktikiqylsdglnrkscsiatvpdgcamycyvstqletddyagsspptqkltlifyndtvter
tispsglegnwativpgvgsgiyfenklifpayggvlpnstigvksareffrpvnpynpcsgpqqdldqralrsyfpsyf
snrriqsaflvcawnqilvtncelvvpssnqtmmgaegrvllinnrllyyqrstswwpyellyeisftftnsgpssvnms
wipiysftrpgsgncsgenvcptacvsgvyldpwpltpyshqsginrnfyftgallnssttrvnptlyvsalnnlkvlap
ygtqglfasyttttcfqdtgdasvycvyimelasnivgefqilpvltrltit

The MeV H ectodomain head dimer (SEQ ID NO: 60), MeV H ectodomain head trimer (SEQ ID NO: 59), and MuV HN ectodomain head trimer (SEQ ID NO: 58) were designed, expressed, purified and characterized by negative stain EM (see FIG. 6A).

Mice were immunized with the purified constructs and sera assessed for MeV and MuV neutralization by PRNT. The immunogens assessed were the MeV H dimer or trimer (SEQ ID NO: 59 or 60), prefusion MuV F ectodomain trimer (MuV F V206C-A223C-GGG-476-GCN4, SEQ ID NO: 11), MuV pre-F-MeV H chimera (MuV F 206C-223C-476+GCN4/Fd+MeV_H_31NB-tHS (SEQ ID NO: 28), and MuV HN trimer (SEQ ID NO: 58).

For MeV neutralization (FIG. 6B), the MeV H dimer elicited PRNT ID₆₀ averaging 66,000, the MeV H trimer 30,000, and the trimerized H on the C-terminus of prefusion MuV F 35,000. These results were quite surprising given the much lower neutralization titers observed from immunization with prefusion MeV F trimer (PRNT of 873; see FIG. 5C). Human PRNT titers following 2× MMR vaccination average approximately 650.

The very high MeV PRNT titer for the MeV H designs was not expected since the prefusion F ectodomain trimer was initially believed to provide a better immune response (similar to other paramyxoviruses, such as RSV). Unexpectedly, the multimeric MeV H immunogens showed very good immunogenicity, being up to 75-fold more potent than prefusion-stabilized MeV F ectodomain trimer and approximately 100-fold higher than human responses following MMR vaccination.

For MuV neutralization (FIG. 6C), each of the assessed immunogens elicited an immune response above the protective threshold, and the soluble trimerized MuV HN elicited a very potent immune response, which is quite surprising given the minimal response elicited by soluble MuV HN monomer (FIG. 4C). Surprisingly, there was a 13.4-fold increase in neutralization potency between the MuV HN ectodomain monomer and the trimeric MuV HN ectodomain.

Protein production, analysis and immunization were performed as noted above.

It will be apparent that the precise details of the methods or compositions described may be varied or modified without departing from the spirit of the described embodiments. We claim all such modifications and variations that fall within the scope and spirit of the claims below.

1. An isolated immunogen, comprising:
a dimer of a MeV H ectodomain head; or
a dimer of a MeV H ectodomain stalk and head.

2. The immunogen of claim 1, wherein:
the MeV H ectodomain head comprises the amino acid sequence set forth as residues 22-459 of SEQ ID NO: 60, or a sequence at least 95% identical to residues 22-459 of SEQ ID NO: 60; or
the MeV H ectodomain stalk and head comprises residues 22-580 of SEQ ID NO: 86, residues 22-577 of SEQ ID NO: 87, residues 22-579 of SEQ ID NO: 88, or residues 22-572 of SEQ ID NO: 89, or a sequence at least 95% identical to residues 22-580 of SEQ ID NO: 86, residues 22-577 of SEQ ID NO: 87, residues 22-579 of SEQ ID NO: 88, or residues 22-572 of SEQ ID NO: 89.

3. The immunogen of claim 1, wherein:
the MeV H ectodomain head comprises or consists of the amino acid sequence set forth as residues 22-459 of SEQ ID NO: 60; or
the MeV H ectodomain stalk and head comprises or consists of the amino acid sequence set forth as residues 22-580 of SEQ ID NO: 86, residues 22-577 of SEQ ID NO: 87, residues 22-579 of SEQ ID NO: 88, or residues 22-572 of SEQ ID NO: 89.

4. The immunogen of claim 1, conjugated to a heterologous carrier.

5. The immunogen of claim 1, wherein the immunogen is soluble.

6. The immunogen of claim 1, wherein the dimer of the MeV H ectodomain head or the dimer of the MeV H ectodomain stalk and head is:
(i) fused to a transmembrane domain by a peptide linker; or
(ii) directly fused to the transmembrane domain.

7. A virus-like particle comprising the immunogen of claim 1.

8. A self-assembling nanoparticle comprising the immunogen of claim 1.

9. A nucleic acid molecule encoding the immunogen of claim 1.

10. A vector comprising the nucleic acid molecule of claim 9.

11. The vector of claim 10, wherein the nucleic acid molecule is operably linked to a promoter.

12. The vector of claim 10, wherein the vector is a plasmid or viral vector.

13. The vector of claim 10, wherein the vector is an RNA vector.

14. A host cell comprising the nucleic acid molecule of claim 9.

15. A method of producing an immunogen, comprising:
expressing the nucleic acid molecule of claim 9 in a host cell, and
purifying the immunogen.

16. The immunogen produced by the method of claim 15.

17. An immunogenic composition, comprising the immunogen of claim 1, a nucleic
acid molecule encoding the immunogen, a vector comprising the nucleic acid molecule, a virus-like particle comprising the immunogen, or a self-assembling protein nanoparticle comprising the immunogen, and a pharmaceutically acceptable carrier.

18. A method of eliciting an immune response to MeV H in a subject, comprising administering to the subject an effective amount of the immunogenic composition of claim 17 to elicit the immune response.

19. The method of claim 18, wherein the immune response inhibits MeV infection in the subject.

20. The method of claim 18, wherein the subject is an adult who was previously immunized with a live attenuated vaccine to MeV and the administration boosts the immune response to the MeV.
